(19) United States
(12) Patent Application Publication
(10) Pub. No.: US 2002/0172987 A1
(43) Pub. Date: Nov. 21, 2002

(54) Methods and reagents for the rapid and efficient isolation of circulating cancer cells

(76) Inventors: Leon W.M.M. Terstappen, Huntingdon Valley, PA (US); Galla Chandra Rao, Princeton, NJ (US); Shawn Mark O'Hara, Ambler, PA (US); Paul A. Liberti, Haverford, PA (US); Steven Gross, Ambler, PA (US); Gerald Doyle, Radnor, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 10/079,939

(22) Filed: Feb. 19, 2002

Related U.S. Application Data
(63) Continuation-in-part of application No. 09/248,388, filed on Feb. 12, 1999, now Pat. No. 6,365,362.
(60) Provisional application No. 60/074,535, filed on Feb. 12, 1998.
(60) Provisional application No. 60/110,279, filed on Nov. 30, 1998.
(60) Provisional application No. 60/110,202, filed on Nov. 30, 1998.
(60) Provisional application No. 60/268,859, filed on Feb. 16, 2001.
(60) Provisional application No. 60/269,270, filed on Feb. 20, 2001.
(60) Provisional application No. 60/269,271, filed on Feb. 20, 2001.

(51) Int. Cl. G01N 33/574

(52) U.S. Cl. 435/7.23

(57) ABSTRACT

Methods and compositions are provided for detecting circulating tumor cells and assessing said cells for alterations in tumor-diathesis associated molecules.

### CROSS REFERENCE TO RELATED APPLICATIONS

This application is a continuation-in-part of U.S. application Ser. No. 09/248,388, filed Feb. 12, 1999, which is incorporated by reference herein. The present application also claims priority to the following U.S. Provisional Applications: 60/074,535, filed Feb. 12, 1998; 60/110,279 filed Nov. 30, 1998; 60/110,202, filed Nov. 30, 1998; 60/268,859, filed Feb. 16, 2001; 60/269,270, and 60/269,271, each filed Feb. 20, 2001. The entire disclosures of all of the foregoing provisional applications are incorporated by reference into the present specification.

### FIELD OF THE INVENTION

This invention relates to the fields of oncology and diagnostic testing. The invention is useful for cancer screening, staging, monitoring for chemotherapy treatment responses, cancer recurrence or the like. More specifically, the present invention provides reagents, methods and test kits that facilitate analysis and enumeration of tumor cells, or other rare cells isolated from biological samples. The invention also provides materials and methods for assessing tumor diathesis associated molecules, such as nucleic acids, proteins and carbohydrates, thereby aiding the clinician in the design therapeutic treatment strategies.

### BACKGROUND OF THE INVENTION

Each year in the United States, approximately 600,000 new cases of cancer are diagnosed; one out of every five people in this country will die from cancer or from complications associated with its treatment. Considerable efforts are continually directed at improving treatment and diagnosis of this disease.

Most cancer patients are not killed by their primary tumor. They succumb instead to metastases: multiple widespread tumor colonies established by malignant cells that detach themselves from the original tumor and travel through the body, often to distant sites. If a primary tumor is detected at an early stage, it can often be eliminated by surgery, radiation, or chemotherapy or some combination of these treatments. Unfortunately, metastatic colonies are frequently more difficult to detect and eliminate and it is often impossible to treat all of them successfully. Therefore, from a clinical point of view, metastasis can be considered the penultimate event in the natural progression of cancer. Moreover, the ability to metastasize is the property that uniquely characterizes a malignant tumor.

Cancer metastasis comprises a complex series of sequential events. These are:

1) extension from the primary locus into surrounding tissues;

2) penetration into body cavities and vessels;

3) release of tumor cells for transport through the circulatory system to distant sites;

4) reinvasion of tissue at the site of arrest; and

5) adaptation to the new environment so as to promote tumor cell survival, vascularization and tumor growth.

Based on the complexity of cancer and cancer metastasis and the frustration in treating cancer patients over the years, many attempts have been made to develop diagnostic tests to guide treatment and monitor the effects of such treatment on metastasis or relapse. Such tests presumably could also be used for cancer screening, replacing relatively crude tests such as mammography for breast tumors or digital rectal exams for prostate cancers. Towards that goal, a number of tests have been developed over the last 20 years. One of the first attempts was the formulation of an immunoassay for carcinoembryonic antigen [CEA]. This antigen appears on fetal cells and reappears on tumor cells in certain cancers. Extensive efforts have been made to evaluate the usefulness of testing for CEA as well as many other tumor antigens, such as PSA, CA 15.3, CA125, PSMA, and CA27.29. However, the appearance of such antigens in blood has not been generally predictive and is often detected when there is little hope for the patient. In the last few years, however, one test has proven to be useful in the early detection of cancer, viz., Prostate Specific Antigen [PSA] for prostate cancers. When used with follow-up physical examination and biopsy, the PSA test has played a remarkable role in detecting prostate cancer early, at the time when it is best treated.

Despite the success of PSA testing, the test leaves much to be desired. For example, high levels of PSA do not always correlate with cancer nor do they appear to be an indication of the metastatic potential of the tumor. This may be due in part to the fact that PSA is a component of normal prostate tissue as well as other unknown factors. Moreover, it is becoming clear that a large percentage of prostate cancer patients will continue to have localized disease which is not life threatening. Based on the desire to obtain better concordance between those patients with cancers that will metastasize and those that won't, attempts have been made to determine whether or not prostate cells are in the circulation. When added to high PSA levels and biopsy data, the existence of circulating tumor cells might give indications as to how vigorously the patient should be treated.

One approach for determining the presence of circulating prostate tumor cells has been to test for the expression of messenger RNA for PSA in blood. This is being done through the laborious procedure of density separation of mononuclear cells from a blood sample, followed by isolating all of the mRNA from these cells, and performing reverse transcriptase PCR for PSA. As of this date, (Gomella LG. J of Urology. 158:326-337 (1997)) no good correlation exists between the presence of such cells in blood and the ability to predict which patients are in need of vigorous treatment. It is noteworthy that PCR is difficult, if not impossible in many situations, to perform quantitatively, i.e., determine number of tumor cells per unit volume of biological sample. Additionally false positives are often observed using this technique. An added drawback is that there is a finite and practical limit to the sensitivity of this technique based on the sample size examined. Typically, the test is performed on 10⁵ to 10⁶ cells purified away from interfering red blood cells. This corresponds to a practical lower limit of sensitivity of one tumor cell/0.1 ml of blood. Hence, approximately 10 tumor cells in a ml of blood must be present before signal is detectable. As a further consideration, tumor cells are often genetically unstable. Accordingly, cancer cells having genetic rearrangements and sequence changes may be missed in a PCR assay as the requisite sequence complementarity between PCR primers and target sequences can be lost.

In summary, a useful diagnostic test needs to be very sensitive and reliably quantitative. If a blood test can be developed where the presence of a single tumor cell can be detected in lml of blood, that would correspond on average to 3000-4000 total cells in circulation. Innoculum studies for establishing tumors in animals show that injection of 3000-4000 of cells can indeed lead to the establishment of a tumor. Further if 3000-4000 circulating cells represent 0.01% of the total cells in a tumor, then it would contain about 4×10⁷ total cells. A tumor containing that number of cells would not be visible by any technique currently in existence. Hence, if tumor cells are shed in the early stages of cancer, a test with the sensitivity mentioned above would detect the cancer. If tumor cells are shed in some functional relationship with tumor size, then a quantitative test would be beneficial to assess tumor burden. Heretofore there has been no information regarding the existence of circulating tumor cells in very early cancers. Further, there are considerable doubts in the medical literature regarding the existence of such cells and the potential of such information. The general view is that tumors are initially well confined and hence there will be few if any circulating cells in early stages of disease. Also, there are doubts that the ability to detect cancer cells early on will provide useful information.

Based on the above, it is apparent that a method for identifying those cells in circulation with metastatic potential prior to establishment of a secondary tumor is highly desirable, particularly early on in the cancer. To appreciate the advantage such a test would have over conventional immunoassays, consider that a highly sensitive immunoassay has a lower limit of functional sensitivity of 10⁻¹⁷ moles. If one tumor cell can be captured from a ml of blood and analyzed, the number of moles of surface receptor, assuming 100,000 receptors per cell would be 10⁻¹⁹ moles. Since about 300 molecules can be detected on a cell such an assay would have a functional sensitivity on the order of 10⁻²² moles, which is quite remarkable. To achieve that level of sensitivity in the isolation of such rare cells, and to isolate them in a fashion which does not compromise or interfere with their characterization is a formidable task.

Many laboratory and clinical procedures employ bio-specific affinity reactions for isolating rare cells from biological samples. Such reactions are commonly employed in diagnostic testing, or for the separation of a wide range of target substances, especially biological entities such as cells, proteins, bacteria, viruses, nucleic acid sequences, and the like.

Various methods are available for analyzing or separating the above-mentioned target substances based upon complex formation between the substance of interest and another substance to which the target substance specifically binds. Separation of complexes from unbound material may be accomplished gravitationally, e.g. by settling, or, alternatively, by centrifugation of finely divided particles or beads coupled to the target substance. If desired, such particles or beads may be made magnetic to facilitate the bound/free separation step. Magnetic particles are well known in the art, as is their use in immune and other bio-specific affinity reactions. See, for example, U.S. Pat. No. 4,554,088 and Immunoassays for Clinical Chemistry, pp. 147-162, Hunter et al. eds., Churchill Livingston, Edinburgh (1983). Generally, any material that facilitates magnetic or gravitational separation may be employed for this purpose. However, it has become clear that magnetic separation means are the method of choice.

Magnetic particles can be classified on the basis of size; large (1.5 to about 50 microns), small (0.7-1.5 microns), or colloidal (<200 nm), which are also referred to as nanoparticles. The third, which are also known as ferrofluids or ferrofluid-like materials and have many of the properties of classical ferrofluids, are sometimes referred to herein as colloidal, superparamagnetic particles.

Small magnetic particles of the type described above are quite useful in analyses involving bio-specific affinity reactions, as they are conveniently coated with biofunctional polymers (e.g., proteins), provide very high surface areas and give reasonable reaction kinetics. Magnetic particles ranging from 0.7-1.5 microns have been described in the patent literature, including, by way of example, U.S. Pat. Nos. 3,970,518; 4,018,886; 4,230,685; 4,267,234; 4,452,773; 4,554,088; and 4,659,678. Certain of these particles are disclosed to be useful solid supports for immunological reagents.

The efficiency with which magnetic separations can be done and the recovery and purity of magnetically labeled cells will depend on many factors. These include:

the number of cells being separated,

the receptor or epitope density of such cells,

the magnetic load per cell,

the non-specific binding (NSB) of the magnetic material,

the carry-over of entrapped non-target cells,

the technique employed,

the nature of the vessel,

the nature of the vessel surface,

the viscosity of the medium, and

the magnetic separation device employed.

If the level of non-specific binding of a system is substantially constant, as is usually the case, then as the target population decreases so will the purity.

As an example, a system with 0.8% NSB that recovers 80% of a population which is at 0.25% in the original mixture will have a purity of 25%. Whereas, if the initial population was at 0.01% (one target cell in 10⁶ bystander cells), and the NSB were 0.001%, then the purity would be 8%. Hence, a high the purity of the target material in the specimen mixture results in a more specific and effective collection of the target material. Extremely low non-specific binding is required or advantageous to facilitate detection and analysis of rare cells, such as epithelial derived tumor cells present in the circulation.

Less obvious is the fact that the smaller the population of a targeted cell, the more difficult it will be to magnetically label and to recover. Furthermore, labeling and recovery will markedly depend on the nature of magnetic particle employed. For example, when cells are incubated with large magnetic particles, such as Dynal beads, cells are labeled through collisions created by mixing of the system, as the beads are too large to diffuse effectively. Thus, if a cell were present in a population at a frequency of 1 cell per ml of blood or even less, as may be the case for tumor cells in very early cancers, then the probability of labeling target cells will be related to the number of magnetic particles added to the system and the length of time of mixing. Since mixing of cells with such particles for substantial periods of time would be deleterious, it becomes necessary to increase particle concentration as much a possible. There is, however, a limit to the quantity of magnetic particle that can be added, as one can substitute a rare cell mixed in with other blood cells for a rare cell mixed in with large quantities of magnetic particles upon separation. The latter condition does not markedly improve the ability to enumerate the cells of interest or to examine them.

Based on the foregoing, high gradient magnetic separation with an external field device employing highly magnetic, low non-specific binding, colloidal magnetic particles is the method of choice for separating a cell subset of interest from a mixed population of eukaryotic cells, particularly if the subset of interest comprises but a small fraction of the entire population. Such materials, because of their diffusive properties, readily find and magnetically label rare events, such as tumor cells in blood. For magnetic separations for tumor cell analysis to be successful, the magnetic particles must be specific for epitopes that are not present on hematopoeitic cells.

### SUMMARY OF THE INVENTION

Once tumor cells are identified in circulation, it is desirable to further characterize the isolated cells phenotypically or biochemically. Thus, particular tumor diathesis associated molecules, such as nucleic acid molecules, proteins, or carbohydrates that are associated with the malignant phenotype may be analyzed. Specifically, methods are provided for measuring the level of expression of predetermined tumor diathesis associated molecules present in or on tumor cells identified in the circulation to assist the clinician in diagnosing the type of cancer and assessing the efficacy of chemotherapeutic intervention strategies.

In a preferred embodiment of the invention, a method for assessing a patient for the presence of a malignancy is provided. The method entails obtaining a biological specimen from a patient comprising a mixed cell population suspected of containing hematopoietic and non-hematopoietic malignant cells. A sample is then prepared wherein the biological specimen is mixed with a detectably labeled ligand which reacts specifically with the malignant cells, to the substantial exclusion of other sample components. The sample is contacted with at least one reagent which also specifically labels said malignant cells. Analysis of the sample is then performed to determine the presence and number of labeled cells, detection of said cells indicating the presence of malignancy, the greater the number of labeled cells present, the greater the severity of the malignancy. The method further comprises assessment of said labeled cells for alterations in at least one tumor diathesis-associated molecule. In one embodiment, this assessment comprises contacting said molecule with a detectably labeled agent having binding affinity therefore. Tumor diathesis associated molecules may be proteins, nucleic acids or carbohydrates and are assessed using conventional methods.

In one aspect of the method, malignant cells are analyzed by a process selected from the group consisting of multiparameter flow cytometry, immunofluorescent microscopy, laser scanning cytometry, bright field base image analysis, capillary volumetry, spectral imaging analysis manual cell analysis, Cell Spotter® analysis, Cell Tracks analysis and automated cell analysis.

The method of the invention may be used to assess residual cancer cell in circulation following medical, radiation or surgical treatment to eradicate the tumor. The method may also be performed periodically over the course of years to assess the patient for the presence and number or tumor cells in the circulation, and alterations in tumor diathesis molecules therein as an indicator of occurrence, recurrence and/or progression of disease.

In yet another aspect of the invention, methods are provided for determining alterations in tumor diathesis associated molecules as a means to predict efficacy of therapy. An exemplary method comprises obtaining a sample from a patient; isolating and enumerating circulating malignant cells from said sample if present, and determining the number of at least one predetermined tumor diathesis associated molecule on individual cells present in said sample as a means to predict efficacy of therapy. Such methods may also be used to advantage to assess the appropriate dosage of a given therapeutic regimen and/or for monitoring the efficacy of therapy over time. Thus, the methods of the invention provide a “whole body” biopsy based on a simple blood test.

In yet another aspect of the invention, methods for culturing tumor cells isolated from the circulation are provided. Such cells may then be contacted with therapeutic agents to assess their sensitivity thereto. Such cells also provide a source for tumor diathesis associated molecules which may or may not be altered. Thus, the present invention also encompasses tumor cells or cultures thereof, isolated from the circulation.

In a preferred aspect of the invention, tumor vaccines derived from the isolated circulating tumor cells of the invention are disclosed. Such tumor vaccines may comprise circulating tumor cells, fragments thereof or purified tumor diathesis associated molecules.

In yet another aspect of the invention, a method for identifying alterations in a circulating tumor cells relative to cells present in a tumor mass in situ is provided. An exemplary method comprises obtaining a biopsy specimen of said tumor mass from patient and isolating circulating tumor cells from said patient, if any are present. Both the specimen and the isolated circulating tumor cells are then contacted with a duplicate panel of agents which detect a plurality of tumor diathesis associated molecules, such agents optionally being detectably labeled. Any tumor diathesis associated molecules present in said circulating tumor cells and in said specimen are then analyzed to determine whether the tumor diathesis associated molecules are altered in said circulating tumor cell relative to said biopsy specimen.

In a further aspect of the present invention, kits are provided for screening a patient sample for the presence of a non-hematopoietic malignant cells. An exemplary kit of the invention comprises coated magnetic nanoparticles comprising i) a magnetic core material, a protein base coating material, and an antibody that binds specifically to a first characteristic determinant of said malignant cell, the antibody being coupled, directly or indirectly, to said base coating material; ii) at least one antibody having binding specificity for a second characteristic determinant of said malignant cell; iii) a cell specific dye for excluding sample components other than said malignant cells from analysis; iv) a device selected from the group consisting of a Cell Spotter® cartridge or a Cell Tracks cartridge; and at least one detectably labeled agent having binding affinity for a tumor diathesis associated molecule. Such kits may optionally comprise an antibody which has binding affinity for non-target cells, a biological buffer, a permeabilization buffer, a protocol and optionally, an information sheet.

### BRIEF DESCRIPTION OF THE DRAWINGS

FIG. 1 is a schematic diagrams showing steps of the sample preparation method of the present invention.

FIGS. 2A-D show various aspects of the CellSpotter® Chamber of the invention. Panel 2A: Chamber and holder containing two yoked angular shaped magnets; Panel 2B Computer simulations of trajectories (indicated by the dashed lines) of cells labeled with magnetic nanoparticles placed randomly in a field created by two angular shaped magnets. Panel 2C Close up of the trajectories of the cells within the CellSpotter® Chamber placed in between the magnets as shown in Panel 2B. Panel 2D Top view of the surface of the chamber. The horizontal lines are magnetic nanoparticles ligned up along the ferromagnetic field lines.

FIGS. 3A-3D are a series of micrographs showing fluorescent images of a frame in a CellSpotter® chamber taken from a blood sample processed from a breast cancer patient. Panel 3A Dapi image showing the nuclei from the internal control, leukocytes and tumor cells. Panel 3B DiOC16 image showing the fluorescence of 5 control cells. Panel 3C CK-PE image showing the fluorescence of 5 control cells and two candidate tumor cells, one bright and one dimly staining. Panel 3D CD45-APC showing the fluorescence of leukocytes and showing no staining of the control cells, the box showing dim PE staining shows APC staining, and the other box showing no APC staining confirming that it contains a CTC.

FIG. 4 shows the classification of tumor cell candidates. Six rows of thumbnails of tumor cell candidates from a breast cancer patient sample. Row 201, 202 and 204 are checked indicating the presence of tumor cells. Thumbnails under Composite are composites of DAPI (purple) and CK-PE staining. L-APC=leukocyte staining with CD45 APC, CNTL=control cell staining with DiOC16, EC-PE=epithelial cell staining with cytokeratin-PE, NADYE=nucleic acid staining with DAPI.

FIG. 5 shows the results of model experiments in which known number of tumor cells are spiked into peripheral blood and retrieved after immunomagnetic selection and analysis by either microscopy (Panel A) or flowcytometry (Panel B).

FIG. 6 shows flowcytometric analysis of cell suspensions obtained after immunomagnetic cell selection from 10 ml of blood from a patient having distant metastasis of carcinoma of the breast, drawn 48, 175 and 300 days after this patient entered the study. After immunomagnetic selection, the cells were stained with an epithelial cell specific phycoerythrin (PE) conjugated monoclonal antibody, a leukocyte specific CD45 PerCP conjugated monoclonal antibody and a nucleic acid dye. Events passing a threshold on the nucleic acid dye were acquired into listmode and 85% of the sample was analyzed. The tumor cells are highlighted and illustrated in black and their number is shown in the top right corner; the background events, consisting of residual leukocytes and debris, are illustrated in gray.

FIG. 7A-H shows epithelial cell number in 10 ml of blood and clinical activity of the disease at different time points for eight patients with active carcinoma of the breast. The clinical activity of the disease was classified in categories 1 through 4, as set out in Table IV. The bars at the top represent the length of time of chemotherapy. Panel A, adriamycin (ADR) 90 and 110 mg/mz² respectively, Panel B, ADR 30 mg/m²/week, Vinorelbine (Vin) 20 mg/m²/week, ADR 160 mg, ADR 20 mg/m²/week, Panel C, vincristine (Vinc) 0.7 mg/m²/week, methotrexate (MTX) 30 mg/m²/week, Panel D, vinblastine (Vinb) 7 mg/m²/week, ADR 20 mg/m²/week, Vinb 6 mg/m²/week, 5-fluoruracil (5FU) 700 mg/m²/week. Panel E, Vin 20 mg/m²/week; 5FU 800 mg/m²/week+Leukovorin 50 mg/m²/week. Panel F, ifosfamide (IF) 18 mg/m²/week; 5FU 850 mg/m²/week +Leukovorin 35 mg/m²/week, 5FU 605 mg/m²/week; Vin 20 mg/m²/week+Leukovorin 30 mg/m²/week. Panel G, Vin 20 mg/m²/week, Panel H, Vin 20 mg/m²/week FIGS. 8A-8D are a series of micrographs showing the results obtained following analysis of immunomagnetically-selected cells from peripheral blood of patients with a history of breast carcinoma. Panel A, cells from a patient three years after surgery (T2N1M0) staining positive for cytokeratin. Panel B, cell from a patient eight years after surgery (T2N1M1) in complete remission stained with Wright Giemsa. Panel C and D cells from a patient 2 years after surgery (T2N0M0) stained with Wright Giemsa. The images were taken with a Pixera digital camera with a 100× objective.

FIGS. 9A-9C are a series of graphs showing the correlation between severity of disease and circulating epithelial cell number in three patients with prostate cancer.

FIGS. 10A-10H show CTC and PSA levels measured at intervals of 0, 1, 2, 7, 12, 17, and 25 weeks in the blood of 8 patients with CAP. No significant change in the clinical activity of the disease during the time course was noted in these patient samples (A-D). However, disease activity increased in these patient samples (E-H). The correlation coefficient R between the CTC count and PSA level for the patient sample in FIG. 10E was 0.42; for the patient sample in FIG. 10F, it was 0.87; for the patient sample in FIG. 10G, it was 0.65; and for the patient sample in FIG. 10H, it was 0.98. Bars on top of the panels indicate hormonal treatment received. In the patient sample in FIG. 10B, the CTC count never rose above the 99% confidence level of the control group.

FIGS. 11 A and 11 B are a pair of graphs showing CTC number and PSA levels measured at intervals of 0, 1, 2, 7, 12, 17, and 25 weeks in the blood of 2 patients with CAP. Bars on top indicate hormonal treatment received. Both patients received chemotherapy; administered drugs and time of administration are indicated with arrows at the bottom of the figure.

FIG. 12 is a graph that shows that circulating epithelial cell number in patients with colon cancer is significantly decreased after surgical removal of the tumor.

FIG. 13 is a graph that shows that circulating epithelial cell number in patients with metastatic disease of the colon increases with the severity and extent of metastatic disease.

FIG. 14 is a schematic diagram showing the progression of cancer from a primary tumor to growing metastases.

FIGS. 15A-15D are four scatter plots showing the levels of CTCs and HER-2⁺-CTC in blood as determined by flowcytometric analysis. Multiparameter flow cytometric analysis of EpCAM ferrofluid selected cells from 5 ml of blood obtained from a patient with breast cancer. Leukocytes and beads are presented as small black dots and their positions are indicated in the panels. The gray dots represent debris. CTCs are the large black dots and the criteria used to identify CTC are indicated by the regions in each of the panels. In the correlative display of cytokeratin versus HER-2 the border above which cells express HER-2 is indicated by a dashed line.

FIGS. 16A-D are a histogram and scatter plots showing quantification of HER-2 density on cell lines and CTCs of 3 breast cancer patients. Panel 16A, HER-2 expression of leukocytes, PC3 cells and SKBR-3 cells immunomagnetically selected from 5 ml of blood and gated on CD45 and cytokeratin expression. The expression levels of HER-2 were subdivided into four categories (−, +, ++, +++), based on the quantitative assessment of HER-2 expression on PC3 and SKBR-3 cells. (−) no expression below 5000 receptors (WBC) (+) expression between 5,000 and 50,000 receptors (PC-3), (++) expression between 50,000 and 500,000 receptors and (+++) expression of more than 500,000 receptors (SKBR-3). Panels 16B, 16C and 16D, shows the expression of cytokeratin and HER-2 on CTCs from three patients (2, 20 and 25 from Table XII with breast cancer. Only the CTCs are shown in the panels.

FIGS. 17A-17C are a series of graphs showing acquisition of HER-2 overexpression during disease progression. CTCs during the course of treatment of three breast cancer patients with HER-2⁻CTCs at baseline and whose disease progressed during follow up. The bars indicate the total number of CTCs at each time point. Within each bar, the number of CTCs that expressed different levels of HER-2 is indicated by [symbol] HER-2⁻, [symbol] HER-2⁺, [symbol] HER-2⁺⁺ [symbol] HER-2⁺⁺⁺.

The days and type of treatment are indicated at the top of the figure. Megestrol acetate shown in Panel 17C was taken daily. HER-2 was not expressed on CTCs before treatment. A change in the phenotype of the CTCs, represented by a conversion to HER-2 positive, was clearly detected in all three patients as the number of CTC increased. Patient numbers refer to Table XII.

FIGS. 18A-18C are a series of graphs showing fluctuation in HER-2 density on CTCs in patients with HER-2⁺ CTCs during disease progression. CTCs during the course of treatment of three breast cancer patients with HER-2⁺ CTCs at baseline and whose diseases progressed during follow up. Other indicators as per FIG. 17. Exemestane shown in Panel 18B was taken daily. A portion of the CTCs expressed HER-2 throughout the course of treatment. CTCs increased substantially during the treatment course of patient 23 and 25.

FIGS. 19A-19C are a series of graphs showing fluctuation in HER-2 density on CTCs in patients with stable disease. CTCs during the course of treatment in three breast cancer patients with HER-2⁺ CTCs at baseline and whose disease remained clinically stable during therapy. Other indicators as per FIG. 17. Anastrozole (Panel 19B) was taken daily. CTCs in the patient in the top panel increased during the treatment course whereas the CTC in the patients in the two bottom panels decreased.

FIG. 20 is an exemplary schema of a protocol for practicing the methods of the present invention.

### DETAILED DESCRIPTION OF THE INVENTION

According to a preferred embodiment, the present invention provides compositions, methods and kits for the rapid and efficient isolation of rare target bioentities from biological samples. The methods described may be used effectively to isolate and characterize tumor cells present in a blood sample while at the same time minimizing the selection of non-specifically bound or entrapped cells.

Cancer staging systems describe how far cancer has spread anatomically and attempt to put patients with similar prognosis and treatment in the same staging group. The concept of stage is applicable to almost all cancers except for most forms of leukemia. Since leukemias involve all of the blood, they are not anatomically localized like other cancers, so the concept of staging is often not applied to this type of cancer. A few forms of leukemia do have staging systems which reflect various measures of how advanced the disease is. For most solid tumors, there are two related cancer staging systems, the Overall Stage Grouping, and the TNM system.

In Overall Stage Groupings (Roman Numeral Staging) system, cases are grouped into four stages denoted by Roman numerals I through IV, or are classified as “recurrent.” In general, stage I, or early stage cancers, are small localized cancers that are usually curable, while stage IV usually represents inoperable or metastatic cancer. Stage II and III cancers are usually locally advanced and/or with involvement of local lymph nodes. Actually, these stages are defined precisely, but the definition is different for each kind of cancer. In addition, it is important to realize that the prognosis for a given stage also depends on what kind of cancer it is, so that a stage II non small cell lung cancer has a different prognosis from a stage II cervical cancer.

As mentioned previously, it is common for cancer to return months or years after the primary tumor has been removed because cancer cells had already broken away and lodged in distant locations by the time the primary tumor was discovered, but had not formed tumors which were large enough to detect at that time. Sometimes a tiny bit of the primary tumor was left behind in the initial surgery which later grows into a macroscopic tumor. Cancer that recurs after all visible tumor has been eradicated, is called recurrent disease. Disease that recurs in the area of the primary tumor is locally recurrent, and disease that recurs as metastases is referred to as a distant recurrence. Distant recurrence is usually treated similarly to stage IV disease (sometimes the terms are used interchangeably). The significance of a local recurrence may be quite different than distant recurrence, depending on the type of cancer.

For solid tumors, stages I-IV are actually defined in terms of a more detailed staging system called the “TNM” system. In this system, TNM stands for Tumor, Nodes, and Metastases. Each of these is categorized separately and classified with a number to give the total stage. Thus a TlNlMO cancer means the patient has a T1 tumor, N1 lymph node involvement, and no distant metastases. Of course the definitions of T, N and M are specific to each cancer, but it is possible to broadly define their meaning.

T: Tumor-Classifies the extent of the primary tumor, and is normally given as T0 through T4. TO represents a tumor that has not even started to invade the local tissues. This is called “In Situ”. T4 on the other hand represents a large primary tumor that has probably invaded other organs by direct extension, and which is usually inoperable. N: Lymph Nodes-Classifies the amount of regional lymph node involvement. It is important to understand that only the lymph nodes draining the area of the primary tumor are considered in this classification. Involvement of distant lymph nodes is considered to be metastatic disease. The definition of just which lymph nodes are regional depends on the type of cancer. N0 means no lymph node involvement while N4 means extensive involvement. In general more extensive involvement means some combination of more nodes involved, greater enlargement of the involved nodes, and more distant (But still regional) node involvement. M: Metastasis-M is either M0 if there are no metastases or M1 if there are metastases. As with the overall staging system, the exact definitions for T and N are different for each different kind of cancer.

Most oncologists consider the TNM system to be more precise than the I through IV system as this system provides more precise categories. However, the two systems are actually related. The I through IV groupings are actually defined using the TNM system. For example, stage II non-small cell lung cancer means a T1 or T2 primary tumor with N1 lymph node involvement, and no metastases (M0).

Many clinicians believe that cancer is an organ-confined disease in its early stages. Based on the data presented herein, it appears that this notion is incorrect. Indeed, the data reveal that cancer is often a systemic disease by the time it is first detected using methods currently available. Hence, the presence of tumor cells in the circulation can be used to screen for cancer in place of, or in conjunction with, other tests, such as mammography, or measurements of PSA. By employing appropriate mononclonal antibodies directed to associated markers on or in target cells, or by using other assays for cell protein expression, or by the analysis of cellular mRNA, the organ origin of such cells may readily be determined, e.g., breast, prostate, colon, lung, ovarian or other non-hematopoietic cancers. Thus, in cases where cancer cells can be detected, while there are essentially no clinical signs of a tumor, it will be possible to identify their presence as well as the organ of origin. Because screening can be done with the relatively simple blood test of the present invention described herein, which functions with a high degree of sensitivity and specificity, the test can be thought of as a “whole body biopsy.” Furthermore, based on the data set forth herein, cancer should be thought of as a blood borne disease characterized by the presence of potentially very harmful metastatic cells, and therefore, treated accordingly. In cases where there is absolutely no detectable evidence of circulating tumor cells, e.g., following surgery, it may be possible to determine from further clinical study whether follow-up treatment, such as radiation, hormone therapy or chemotherapy is required. Predicting the patient's need for such treatment, or the efficacy thereof, given the costs of such therapies, is a significant and beneficial piece of clinical information.

It is also clear from the present data that the number of tumor cells in the circulation is related to the stage of progression of the disease, from its inception to the final phases of disease.

The term “target bioentities” as used herein refers to a wide variety of materials of biological or medical interest and can be distinguished from “non-target” materials that are present in the specimen. Examples include hormones, proteins, peptides, lectins, oligonucleotides, drugs, chemical substances, nucleic acid molecules, (e.g., RNA and/or DNA) and particulate analytes of biological origin, which include bioparticles such as cells, viruses, bacteria and the like. In a preferred embodiment of the invention, rare cells, such as fetal cells in maternal circulation, or circulating cancer cells may be efficiently isolated from non-target cells and/or other bioentities, using the compositions, methods and kits of the present invention.

The term “biological specimen” includes, without limitation, cell-containing bodily fluids, including without limitation, peripheral blood, tissue homogenates, nipple aspirates, colonic lavage, sputum, bronchial lavage, and any other source of cells that is obtainable from a human subject. An exemplary tissue homogenate may be obtained from the sentinel node in a breast cancer patient. The term “determinant”, when used in reference to any of the foregoing target bioentities, refers broadly to chemical mosaics present on macromolecular antigens that often induce an immune response. Determinants may also be used interchangeably with “epitopes”. Determinants may be specifically bound by a biospecific ligand or a biospecific reagent, and refers to that portion of the target bioentity involved in, and responsible for, selective binding to a specific binding substance (such as a ligand or reagent), the presence of which is required for selective binding to occur. In fundamental terms, determinants are molecular contact regions on target bioentities that are recognized by agents, ligands and/or reagents having binding affinity therefor, in specific binding pair reactions.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin and virus-receptor interactions. “Gene specific probing” refers to methods wherein nucleic acid molecules which are complementary to tumor diathesis associated molecules are used to detect the presence or absence of such molecules. Such nucleic acids may or may not be detectably labeled. Various other determinant-specific binding substance combinations are contemplated for use in practicing the methods of this invention, and will be apparent to those skilled in the art. The phrase “tumor diathesis” is used herein to refer to a constitutional susceptibility or predisposition to malignant disease. Predisposition or susceptibility to malignant disease may be inherited, or due to somatic cell mutations that lead to dysregulated cellular proliferation. The phrase “tumor diathesis associated molecule” refers to intracellular and extracellular molecules that are altered biochemically or expressed aberrantly as a cell progresses from a normal to malignant phenotype. Such molecules include without limitation, hormones and hormone regulated proteins, oncogenes, tumor suppressor proteins, apoptosis associated molecules, cell cycle and proliferation associated molecules, carbohydrate molecules associated with malignancy, cytoskeletal proteins and proteins involved in maintenance of cell-to-cell contacts. Methods for analyzing tumor diathesis associated molecules, including proteins, nucleic acids and carbohydrates can be found in Current Protocols in Molecular Biology, F. M Ausubel et al. eds. John Wiley & Sons, Inc. NY, N.Y. (1999). Assessment of altered expression levels or altered molecular structure of tumor diathesis associated molecules provides the clinician with valuable information to aid in the design of treatment and monitoring strategies.

The phrase “malignant cell” refers to a cell which is biochemically and/or phenotypically altered such that normal stringent control of cellular proliferation and/or localization is lost. Malignant cells are not normally present in circulation.

The term “antibody” as used herein, includes immunoglobulins, monoclonal or polyclonal antibodies, immunoreactive immunoglobulin fragments such as F(ab), and single chain antibodies (sfV). Also contemplated for use in the invention are peptides, oligonucleotides or a combination thereof which specifically recognize determinants with specificity similar to traditionally generated antibodies. As mentioned previously, complementary nucleic acids are encompassed within the meaning of “specific binding pair”. The term “detectably label” is used to herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

The phrase “to the substantial exclusion of” refers to the specificity of the binding reaction between the biospecific ligand or biospecific reagent and its corresponding target determinant. Biospecific ligands and reagents have specific binding activity for their target determinant yet may also exhibit a low level of non-specific binding to other sample components.

The phrase “early stage cancer” is used interchangeably herein with “Stage I” or “Stage II” cancer and refers to those cancers that have been clinically determined to be organ-confined. Also included are tumors too small to be detected by conventional methods such as mammography for breast cancer patients, or X-rays for lung cancer patients. While mammography can detect tumors having approximately 2×10⁸ cells, the methods of the present invention should enable detection of circulating cancer cells from tumors approximating this size or smaller.

The term “enrichment” as used herein refers to the process of substantially increasing the ratio of target bioentities (e.g., tumor cells) to non-target materials in the processed analytical sample compared to the ratio in the original biological sample. In cases where peripheral blood is used as the starting materials, red cells are not counted when assessing the extent of enrichment. Using the method of the present invention, circulating epithelial cells may be enriched relative to leucocytes to the extent of at least 2,500 fold, more preferably 5,000 fold and most preferably 10,000 fold.

The phrase “clonal expansion” when used in reference to isolated, circulating tumor cells, refers to methods of placing the isolated cells in culture under conditions whereby the cells proliferate. Single cells may be cultured such that they form colonies which may then be clonally expanded to generate a population of essentially homogeneous cancer cells. Portions of such cells or the cells themselves may be used to generate tumor vaccines. The phrase “tumor vaccine” as used herein refers to agents that contain a specific protein of the tumor cell that can be used to stimulate an immune response. Vaccines can comprise viruses, small proteins, or whole cells. Methods for generating tumor vaccines using tumor cells infected with an adenovirus-associated vector are disclosed in U.S. Pat. No. 6,171,597. Additional methods for generating tumor vaccines from circulating tumor cells are disclosed in U.S. Pat. No. 5,993,829.

The preferred magnetic particles for use in carrying out this invention are particles that behave as colloids. Such particles are characterized by their sub-micron particle size, which is generally less than about 200 nm (0.20 microns), and their stability to gravitational separation from solution for extended periods of time. In addition to the many other advantages, this size range makes them essentially invisible to analytical techniques commonly applied to cell analysis. Particles within the range of 90-150 nm and having between 70-90% magnetic mass are contemplated for use in the present invention. Suitable magnetic particles are composed of a crystalline core of superparamagnetic material surrounded by molecules which are bonded, e.g., physically absorbed or covalently attached, to the magnetic core and which confer stabilizing colloidal properties. The coating material should preferably be applied in an amount effective to prevent non-specific interactions between biological macromolecules found in the sample and the magnetic cores. Such biological macromolecules may include carbohydrates such as sialic acid residues on the surface of non-target cells, lectins, glyproteins, and other membrane components. In addition, the material should contain as much magnetic mass per nanoparticle as possible. The size of the magnetic crystals comprising the core is sufficiently small that they do not contain a complete magnetic domain. The size of the nanoparticles is sufficiently small such that their Brownian energy exceeds their magnetic moment. As a consequence, North Pole, South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in U.S. Pat. Nos. 4,795,698, 5,597,531 and 5,698,271. Their preparation from those base materials is described below.

Malignant tumors are characterized by their ability to invade adjacent tissue. In general, tumors with a diameter of 1 mm are vascularized and animal studies show that as much as 4% of the cells present in the tumor can be shed into the circulation in a 24 hour period (Butler, TP & Gullino PM, 1975 Cancer Research 35:512-516). The shedding capacity of a tumor is most likely dependent on the aggressiveness of the tumor. Although tumor cells are shed into the circulation on a continuous basis, it is believed that none or only a small fraction will give rise to distant metastasis (Butler & Gullino, supra). Using the following assumptions, one can approximate the frequency of tumor cells in circulation as follows:

1. A tumor with a diameter of 1 mm contains 10⁷ cells, and 4% or 4×10⁵ cells will be shed into the circulation in a 24 hour period;

2. tumor cells only survive one circulatory cycle;

3. a blood volume of about 5 liters; and

4. a cardiac output of 5000 ml/minute.

In such a case, the frequency of tumor cells in peripheral blood of a patient with a lmm diameter tumor is approximately 6 tumor cells/100 ml of blood. Increase in tumor mass might be expected to be proportional to an increase in the frequency of the circulating tumor cells. If this were found to be the case, methods available with this level of sensitivity would facilitate assessment of tumor load in patients with distant metastasis as well as those with localized disease. Detection of tumor cells in peripheral blood of patients with localized disease has the potential not only to detect a tumor at an earlier stage but also to provide indications as to the potential invasiveness of the tumor.

Several studies report the presence of carcinoma cells in leukopheresis products harvested from patients with carcinoma of the breast for autologous peripheral blood stem cell transplantation (Brugger W, et al. (1994) Blood 83:636-640; Brockstein BE, et al. (1996) J of Hematotherapy 5:617; Ross AA, et al. (1993) Blood 82:2605; Ross AA. (1998) J of Hematotherapy. 7:9-18; Moss TJ, et al. (1994) J. Hematotherapy. 3:163-163). These findings prompted criticism of the use of this procedure for autologous transplantation since the tumor cells in the transplant product have the potential to establish metastasis. Additionally, it was found that leukopheresis products were more likely to contain tumor cells when obtained from individuals with disseminated disease (Brugger et al., 1994, supra). These studies, however, do not report quantitative data, nor do they report that tumor cells can be found in peripheral blood of patients with localized disease. Given these observations, one may hypothesize that a highly sensitive and quantitative test that counts the number of tumor cells in peripheral blood may be used to determine actual tumor load. To assess the feasibility of such testing, a sensitive cellular assay was developed which allows precise enumeration of circulating carcinoma cells that is limited only by the blood volume to be tested.

It should be noted that a number of different cell analysis platforms can be used to identify and enumerate cells in the enriched samples. Examples of such analytical platforms are Immunicon's CellSpotter® system, a magnetic cell immobilization and analysis system, using microscopic detection for manual observation of cells described in Example II, and the CellTracks system, an a more advanced automatic optical scanning system, described in U.S. Pat. Nos. 5,876,593; 5,985,153 and 6,136,182 respectively. All of the aforementioned U.S. Patent Applications are incorporated by reference herein as disclosing the respective apparatus and methods for manual or automated quantitative and qualitative cell analysis. Such devices may be used to advantage in the diagnostic and monitoring kits of the present invention.

Other analysis platforms include laser scanning Cytometry (Compucyte), bright field base image analysis (Chromavision), and capillary Volumetry (Biometric Imaging).

The enumeration of circulating epithelial cells in blood using the methods and compositions of a preferred embodiment of the present invention is achieved by immunomagnetic selection (enrichment) of epithelial cells from blood followed by the analysis of the samples by multiparameter flowcytometry. The immunomagnetic sample preparation is important for reducing sample volume and obtaining a 104 fold enrichment of the target (epithelial) cells. The reagents used for the multiparameter flowcytometric analysis are optimized such that epithelial cells are located in a unique position in the multidimensional space created by the listmode acquisition of two light scatter and three fluorescence parameters. These include

1) an antibody against the pan-leukocyte antigen, CD45 to identify leucocytes (non-tumor cells);

2) a cell type specific or nucleic acid dye which allows exclusion of residual red blood cells, platelets and other non-nucleated events; and

3) a biospecific reagent or antibody directed against cytokeratin or an antibody having specificity for an EpCAM epitope which differs from that used to immunomagnetically select the cells.

It will be recognized by those skilled in the art that the method of analysis of the enriched tumor cell population will depend on the intended use of the invention. For example, in screening for cancers or monitoring for recurrence of disease, as described hereinbelow, the numbers of circulating epithelial cells can be very low. Since there is some “normal” level of epithelial cells, (very likely introduced during venipuncture), a method of analysis that identifies epithelial cells as normal or tumor cells is desirable. In that case, microscopy based analyses may prove to be the most accurate. Such examination might also include examination of morphology, identification of known tumor diathesis associated molecules (e.g., oncogenes). Suitable tumor diathesis associated molecules that may be further analyzed in accordance with the methods of the invention are provided in Example 11.

Alternatively, in disease states wherein the number of circulating epithelial cells far exceeds that observed in the normal population, an analytical method that enumerates such cells should be sufficient. The determination of patient status according to the methods described herein is made based on a statistical average of the number of circulating rare cells present in the normal population. Levels of circulating epithelial cells in the early stage cancer patient and in patients with aggressive metastatic cancer can also be statistically determined as set forth herein.

The following examples are provided to facilitate the practice of the present invention. These examples are not intended to limit the scope of the invention in any way.

### EXAMPLE 1

Formulation of improved magnetic nanoparticles for the efficient isolation of rare cells from whole blood

Rare cells (e.g., tumor cells in patients with epithelial derived tumors, fetal cells in maternal blood or the like) can be present in frequencies below one rare cell per ml of blood. The number of blood smears required to detect such rare cells is prohibitively large. Assuming 10 rare cells in 10 ml of blood, which corresponds to 10 tumor cells in 5-10×10⁷ white blood cells (leukocytes), cells can be transferred to a microscope slide by cytocentrifugation or by settling, stained with an antibody specific for the rare cells of interest and read manually or automatically. The maximum number of cells that can be transferred to one slide is about 500,000 cells which means 100-200 slides are required to process 10 ml of blood. The time required for analysis by this approach makes it impractical and economically unfeasible. Consequently, enrichment methods such as sample volume reduction and removal of erythrocytes and platelets by density gradient separation or erythrocyte lysis procedures are used for isolating rare cells so as to significantly reduce the number of slides to be analyzed. As noted above, magnetic enrichment is the preferred method for cell separations and, ideally, the nanoparticles employed for this purpose should not have to be removed prior to analysis. Accordingly, the nanoparticles should be small enough so as not to interfere with analytical measurements, i.e. below about 250 nm. Most preferably, the nanoparticles are below 220 nm so as to make them filter sterilizable. Furthermore, the nanoparticle should be large enough and magnetically responsive enough to permit cell separation from simple laboratory tubes, i.e., test tubes, centrifuge tubes, vacutainers and the like in external gradient magnetic separators. Again, as previously noted internal gradient devices are cumbersome, costly and inefficient for the recovery of rare cells. Also, the nanoparticles and magnetic device should give high and reproducible recovery with low non-specific binding. U.S. Pat. No. 5,597,531 describes the synthesis of highly magnetic particles, referred to as direct coated (DC) particles which have many of these characteristics. These nanoparticles are composed of quasispherical agglomerates of crystalline magnetite or other magnetic oxides which are coated with polymers or proteins (based coated magnetic particles). Because of their structure (magnetic core and polymer coat where the core diameter is >>> than the thickness of the coat) they are about 80-85% magnetic mass. The non-specific bindings of these nanoparticles are in the range of 5-8% and they are, therefore, not very practical for rare cell separations. Thus if one is enriching cells present at one cell per ml then at 80% capture efficiency, the best result to be expected using 10 ml of whole blood (considering leukocytes alone) would be 8 cells recovered in a total of 4 million, i.e. a 16-17 fold enrichment. The magnetic particles described in U.S. Pat. No. 5,597,531 do, however, have the appropriate magnetic properties to perform separations with open field separators and from simple laboratory tubes. Further, their mean size is well under the limit suggested above and, hence, they do not interfere with various analytical procedures. Based on extensive studies with those materials, the major contributing factor to non-specific binding to cells was discovered to be the presence of bare crystalline iron oxides on the nanoparticles due to incomplete coating. Such incompletely coated crystals have a sufficiently high positive charge at physiological pH that they are very likely to bind strongly to biological macromolecules, such as negatively charged sialic acid on cell surfaces. An improved method for making particles is described in U.S. Pat. No. 5,698,271. These materials are an improvement over those disclosed in the >531 patent in that the process includes a high temperature coating step which markedly increases the level of coating. Nanoparticles made with bovine serum albumin (BSA) coating using this process, for example, have a 3-5-fold lower non-specific binding characteristic for cells when compared to the DC-BSA materials of U.S. Pat. No. 5,579,531. This decrease in non-specific binding has been shown to be directly due to the increased level of BSA coating material. When such nanoparticles were treated so as to remove BSA coating, non-specific binding returns to high levels. It was thus determined that a direct relationship exists between the amount of BSA coated on iron oxide crystal surfaces and the nonspecific binding of cells. Typically, the non-specific binding of cells from whole blood with these particles was 0.3%, which is significantly better than those, produced from U.S. Pat. 5,579,531. Thus, from 10 ml of whole blood there would be about 200,000 non-target cells that would also be isolated with the cells targeted for enrichment.

In addition to the non-specific binding problem, to be addressed further below, it was found that when different lots of magnetic particles, manufactured as described in U.S. Pat. Nos. 5,579,531 and 5,698,271 were used in rare cell depletions or enrichments, recoveries were inconsistent. Sometimes recoveries were 85-95% and other times they could be 40-50% using the same model system. As the process for manufacturing these materials results in a size dispersion of considerable range (30 nm to 220 nm), it was suspected and confirmed that the size distribution and particularly the presence of small nanoparticles markedly affected target recovery. Since small nanoparticles (30 to 70 nm) will diffuse more readily they will preferentially label cells compared with their larger counterparts. When very high gradients are used, such as in internal gradient columns, the performance of these materials, regardless of size, makes little difference. On the other hand, when using external gradients, or gradients of lesser magnitude than can be generated on microbead or steel wool columns, the occupancy of small nanoparticles on cells has a significant effect. This was conclusively shown to be the case by fractionating DC nanoparticles and studying the effects on recovery. Based on these studies and other optimization experiments, means for fractionating nanoparticles magnetically or on columns was established where base coated magnetic particles could be prepared that were devoid of excessively small or large nanoparticles. For example, base coated particles of mean diameter 100 nm can be produced which contain at best trace amounts of material smaller than 80 nm or over 130 nm. Similarly material of about 120 nm can be made with no appreciable material smaller than 90-95 nm and over 160 nm. Such materials performed optimally with regard to recovery and could be made sub-optimal by the inclusion of 60-70 nm nanoparticles. The preferred particle size range for use in practicing this invention is 90-150 nm for base coated magnetic particles, e.g., BSA-coated magnetite. Particles falling within this preferred range may be obtained using the procedure described by Liberti et al. In Fine Particles Science and Technology, 777-90, E. Pelizzetti (ed.) (1996).

To further address the non-specific binding problem, several routes for making antibody conjugated direct nanoparticles were attempted. Monoclonal antibody specific for rare cells can be directly coupled to, for example, the BSA base coating on the DC magnetic particles by standard heterobifunctional chemistry (referred to herein as direct coupling method). Heterobiofunctional linkers used for these purposes include sulfosuccinimidil-4-[maleimidomethyl]cyclohexane-1-carboxylate (SMCC). In another approach, biotinylated monoclonal antibodies can be coupled to streptavidin that has been coupled to the base coated particles. This conjugate method is referred to herein as a piggyback method. In this process, streptavidin is coupled to the base coated magnetic particles by the same chemistry as the direct coupling method. In one piggyback coupling method, monobiotinylated antibody is allowed to react with streptavidin magnetic particles for 1 hour and then the remaining streptavidin binding sites quenched with free biotin. It is important to quench the remaining streptavidin sites after antibody coupling to prevent binding of any biotinylated antibody to magnetic particles during isolation of rare cells or the cell analysis step. Furthermore, it has been shown that this means for quenching streptavidin is effective for counteracting non-specific binding. Incubation of such materials under a variety of conditions with biotinylated fluorescent macromolecules results in no bound fluorescence. For comparison, anti-EpCAM antibody (GA73.3 obtained from the Wistar Institute, Philadelphia, /Pa.) was coupled to magnetic particles by both methods. Both magnetic particles were then compared for the selection of cells from the colon tumor cell line (Colo-205) spiked into whole blood as well as for the non-specific binding (NSB) or carry-over of leukocytes. The leukocytes present in the final sample were a combination of leukocytes non-specifically bound to magnetic particles and carry-over of cells from the wash steps. Note that following magnetic separation, it is necessary to wash away any cells which were in contact with the tube at the start of the separation or that were transported non-magnetically during the separation process. Table I shows the comparison of those two magnetic particles.

TABLE I

| | Recovery of | |
|---|---|---|
| Magnetic | spiked Colo-205 | NSB and carry over |
| particles | cells (%) | leukocytes (%) |
| EpCAM antibody | 78-82 | 0.1-0.3 |
| directly coupled | | |
| to magnetic | | |
| particles | | |
| (lot.#120325-1) | | |
| EpCAM antibody | 67-78 | 0.05-0.1 |
| coupled to | | |
| magnetic | | |
| particles by | | |
| piggyback method | | |
| (lot.#120607-2) | | |

The first thing noted is that merely coupling antibody or Streptavidin to BSA base particles significantly reduces non-specific binding (data not shown). This is believed to be due to decreasing the accessability of “bare” crystal surfaces to cells for binding. The above table demonstrates that the recovery of spiked cells is comparable for both types of magnetic particles. However, the non-specific binding of leukocytes was 3-fold higher when using the direct antibody coupled magnetic particles. This difference, albeit relatively small, becomes significant when a large volume of blood is processed and analyzed. A reasonable explanation based on many supporting observations for the difference between the two types of magnetic particles is that there are more layers of protein on magnetic particles synthesized using the piggyback coupling method. The surfaces of the magnetic crystals are thus coated more extensively with multiple layers of protein and appear to be sterically “protected”. This prevents binding of non-target cells to the magnetic particles.

In the piggyback coupling method, a limited number of streptavidin binding sites on the magnetic particles are occupied with biotin-antibody and the remainder are saturated with free biotin by the quench process described above. In yet another coupling method, the excess streptavidin binding sites were quenched and saturated with monobiotin-BSA instead of free biotin. The rationale for this approach is that quenching with monobiotin BSA should further sterically inhibit cells from coming in contact with uncoated regions of the nanoparticles, i.e. give better coverage of the nanoparticles. It was shown by carbon analysis that this process increases the amount of protein coupled to the particles. The two magnetic particle preparations were compared in experiments assessing recovery of spiked Colo 205 from whole blood and for non-specific binding of leukocytes. The results are presented in Table II.

TABLE II

| | Recovery of | NSB and carry |
|---|---|---|
| | Colo 205 | over leukocytes |
| Magnetic particles | cells (%) | (%) |
| EpCAM antibody | 93 | 0.08 |
| coupled magnetic | 87 | 0.1 |
| particles B quenched | 85 | 0.1 |
| excess streptavidin | | |
| sites with free | | |
| biotin | | |
| (lot. #131022-1) | | |
| EpCAM antibody | 87 | 0.01 |
| coupled magnetic | 83 | 0.03 |
| particles B quenched | 85 | 0.02 |
| excess streptavidin | | |
| sites with biotin- | | |
| BSA | | |
| (lot. #131022-2) | | |

Monobiotin-BSA may be prepared by conjugating a limited amount of biotin to BSA, such that 30- 40% of the resultant product has no bound biotin.

In summary, magnetic particles having a homogeneous size distribution and biotin-BSA quenched streptavidin binding sites performed extremely well in the assay methods of the present invention. A good recovery of the spiked epithelial tumor cells and almost an order of magnitude reduction in nonspecific binding is obtained using these particles, compared with the biotin-blocked nanoparticles. Thus, these materials and the results obtained with them define a very useful product that can be further optimized. The improved ferrofluid product is made as magnetic as possible, is coated so as to exclude all possible interactions of the magnetic core with any substances in blood including cells (presumably coated with a nonporous monolayer) and are well defined in its size range and distribution. In the preferred situation, a coat material is used which does not interact with biological materials. Where such interactions are unavoidable, a means for blocking them is required. For a material to be as magnetic as possible those produced as described in U.S. Pat. Nos. 5,579,531 and 5,698,271 are preferred starting materials. They are preferable because they are composed of large magnetic cores with an apparent but not complete monolayer of base coating material. For a 100 nm nanoparticle coated with BSA, the core will be about 90 nm of an appropriate magnetic oxide such as magnetite. Such nanoparticles because of the relative size of the cores and coat material are clearly as magnetic as is possible. This is apparent if one considers that the function of the coating is to keep the nanoparticles from undesired interactions with each other, which would lead to macroscopic agglomeration. The coating also promotes sufficient interactions with solvent molecules so as to maintain colloidal behavior and provides a convenient chemical means for coupling. The nanoparticles of U.S. Pat. Nos. 5,579,531 and 5,698,271 are also preferred as a starting material as they have sufficient monolayer coating wherein “holes” in the monolayer can be filled in several ways, viz., sterically and physically. Clearly any coating that promotes the effective complete coverage of the magnetic core, so as to inhibit interactions of the core material with blood components or any other non-specific effects in any other system would be suitable. The less mass such a coating might add to the nanoparticles the better, so as to maximize the magnetic mass to nanoparticle mass ratio.

### EXAMPLE 2

Semi-Automated Sample Preparation and Analysis for Identification of Circulating Tumor Cells

A semi-automated system was developed that processes and analyzes 7.5 ml of blood for the presence of epithelial derived tumor cells. Cells of epithelial cell origin are immunomagnetically labeled and separated from blood. The magnetically captured cells are differentially fluorescent labeled and placed in an analysis chamber. Four-color fluorescent imaging is used to differentiate between debris, hematopoeitic cells and circulating tumor cells (CTC) of epithelial origin. An algorithm is applied on the captured images to enumerate an internal control and identify all objects that potentially classify as tumor cell based on size and immunophenotype. Thumbnail images of each object are presented in an user interface from which the user can determine the presence of tumor cells. In processing the blood of normal donors the internal control showed consistent and reproducible results between systems and operators. CTC were detected in blood samples of patients with metastatic breast cancer, however, other diseases may be analyzed with the system.

As mentioned previously, tumor cells can be present in blood of carcinoma patients at extremely low frequencies (<10 cells per ml). The laborious manual sample preparation and complex analysis methods involved in detecting the presence of CTC often lead to erroneous results. For highly complex laboratory procedures, the root causes of erroneous results can frequently be traced to the cumulative effects of systematic and/or random pre-analytical errors, i.e. errors occurring during sample preparation or pre-processing stages rather than in the analytical method itself. Pre-analytical errors may manifest as variations due to technique-sensitive process steps as well as random or systematic variations from operator to operator. Thus, manual sample preparation in rare cell analysis, when performed inconsistently, can result in high variability and unreliable assay results. Hence, automating such pre-analytical steps minimizes variability and provides more consistent analytical results. Analytical methods frequently used for analyzing the prepared samples are flowcytometry or microscopy. While flowcytometry is sensitive and reproducible, the investigator cannot confirm that an immunophenotypically identified rare event indeed shows morphological features consistent with a tumor cell. Microscopy adds to the confidence in determining malignancy, but has the disadvantage that considerable and variable cell losses are associated with processing of the sample. Here we introduce a novel cell presentation device and method allowing efficient collection and analysis of CTC in a semi-automated four-color fluorescent microscope system.

The following materials and methods are provided to facilitate the practice of Example 2.

The system is primed with System Buffer (Immunicon Corp, Huntingdon Valley, Pa.) that is also used in various steps during the procedure. This buffer consists of phosphate buffered saline, EDTA, and proteins to reduce nonspecific binding of reagents to cells and to system components. Magnetic nanoparticles are coupled to monoclonal antibodies (mabs) specific for epithelial cell adhesion molecule (EPCAM) as described in Example 1. The EpCAM antigen is expressed on cells of epithelial origin, but not on cells of hematopoietic origin (Momburg et al. Cancer Research (1987) 47:2883-2891; Gaffey et al. Am. J. Surg. Path. (1992) 16:593-599; Herlyn et al. J. Immun. Meth. (1984) 73:157-167; De Leij et al. Int. J. Cancer Suppl. (1994) 8:60-63). In addition to anti-EpCAM antibody, desthiobiotin is coupled to the magnetic nanoparticles to form CA-EpCAM, a reagent permitting controlled aggregation by adding soluble streptavidin to cross-link multiple magnetic nanoparticles on CTC, thereby increasing the magnetic loading and capture efficiency of the cells (Liberti et al. (2001) Journal of Magnetism and Magnetic Materials 225: 301-307). Streptavidin in AB buffer (System buffer with streptavidin added) is added to the specimens before the addition of CA-EpCAM to form aggregates, which minimize differences in magnetic capture efficiencies of cells with different EpCAM antigen densities. The magnetically captured cells are fluorescently labeled with anti-cytokeratin conjugated to Phycoerythrin (CK-PE) and anti-CD45 conjugated to Allophycocyanin (CD45-APC) in addition to the nucleic acid specific dye DAPI (4,6-diamidino-2-phenylindole). The anti-cytokeratin recognizes keratins 4,6,8,10,13, and 18, present in cells of epithelial origin. The mabs are added into a buffer medium that contains a detergent to permeabilize the cytoplasmic membrane of the cells (Immuniperm). The final resuspension buffer in Cellfix (Immunicon Corp, Huntingdon Valley, Pa.) is phosphate buffered saline that contains biotin and a cell stabilizer. Biotin, by virtue of its higher affinity for streptavidin, serves to displace desthiobiotin from streptavidin, thereby reversing the controlled cross linking between desthiobiotin on the ferrofluid particles and streptavidin in the aggregates formed in the earlier steps of the assay.

To monitor the accuracy of the procedure, a known number of internal control cells are added to the blood before processing. Internal control cells are the subject of U.S. patent application Ser. No. 09/801,471, the entire disclosure of which is incorporated by reference herein. These control cells can be successfully derived from the cancer tumor cell-lines. As described herein, cells from the breast cancer line, SKBR-3, are stabilized and uniquely labeled with the fluorescent membrane dye DiOC16 (from Molecular Probes) to permit differentiation from endogenous tumor cells. Approximately 1000 control cells are spiked into each specimen. The control cells express EpCAM and are captured concurrently with the tumor cells. Control cells also express intracellular cytokeratin and staining with CK-PE verifies the quality of this reagent. The percent recovery of added control cells provides an indicator of total reagents and system performance for each specimen, unlike external controls that can detect only systematic errors.

Sample Preparation System

The system is equipped with two magnetic separators each consisting of a set of four rectangular rare earth magnets arranged in a quadrupole configuration with a 17 mm diameter cavity surrounded by a circular steel yoke. In the present system, each separator can hold a 15 ml conical tube. In other system arrangements, different tubes may be used with different separators. Adjacent to each separator is a magnetic yoke that holds the analysis chamber (CellSpotter® chamber) into which the final sample is transferred. This chamber assembly can be removed from the system and placed onto the microscope stage. The tube transport consists of two movable tube arms, for raising and lowering the tubes into and out of the magnetic separators, along with a rotary turntable for positioning the tubes at various process positions. Additionally, a probe washbowl is mounted on the turntable allowing internal and external washing of the aspiration and transfer probes. A Cavro XE1000 digital syringe pump with 5 mL syringe is used for fluid deliveries transfers and probe washing. A Cavro SP Smart Peristaltic pump with PharMed tubing is used for aspirations to waste. Two pinch valves and one 3-way valve (Bio-Chem) are used to control fluid paths. Separate aspiration and transfer probes fabricated from 13 AWG Inconel tubing (non-magnetic) are used for fluid access to the 15 ml conical tubes and the chamber. A through-beam photoelectric sensor (Omron) is used for determining the height of the packed red-cell layer to allow precisely controlled plasma aspiration. The system is controlled using an 8-bit microcontroller running firmware to execute the motion controls, process controls, and the operator interface commands. The protocol itself (incubation times, fluid processing steps, etc.) is encoded into a separate memory chip. The operator interface consists of a four-by-five key keypad, a 2-line by 20-character LCD display, and an audible alarm.

CellSpotter® Chamber

A molded chamber with an upper surface planar viewing area of 29.7×2.7 mm and a depth of 4 mm (approximately 3201 volume) is used to collect the prepared sample. The port of the chamber is sealed with a plug designed so that the chamber filling can be performed reproducibly and efficiently, positioning 99% of the sample in the viewing area of the chamber. Special calibration chambers, with registration marks in the center of the chamber, are also used to allow calibration of the chamber center offset from the microscope stage home position and the illumination source.

CellSpotter® System

The CellSpotter® system utilizes a Nikon E-400 microscope equipped with a 10× objective (WD 4 mm, NA 0.45), a high resolution X, Y, Z stage and a filter cube changer control that are controlled with a Ludl MAC2002 controller, which in turn, is controlled by a PC via RS-232. Excitation, dichroic and emission filters in each of four cubes are for DAPI 365 nm/400 nm/400 nm, for DiOC16 480 nm/ 495 nm/ 510 nm, for PE 546 nm/ 560 nm/ 580 nm and for APC 620 nm/ 660 nm/ 700 nm. Images are acquired with a Hamamatsu 12 bit, 1280×1024 pixel digital camera connected to a National Instruments PCI-1424 digital frame grabber. LabVIEW® and the IMAQ Vision Toolkit® were used to develop the data acquisition software. The data analysis and presentation program was written using IBM's DB-2 database through an HTML interface.

Semi automated sample preparation system

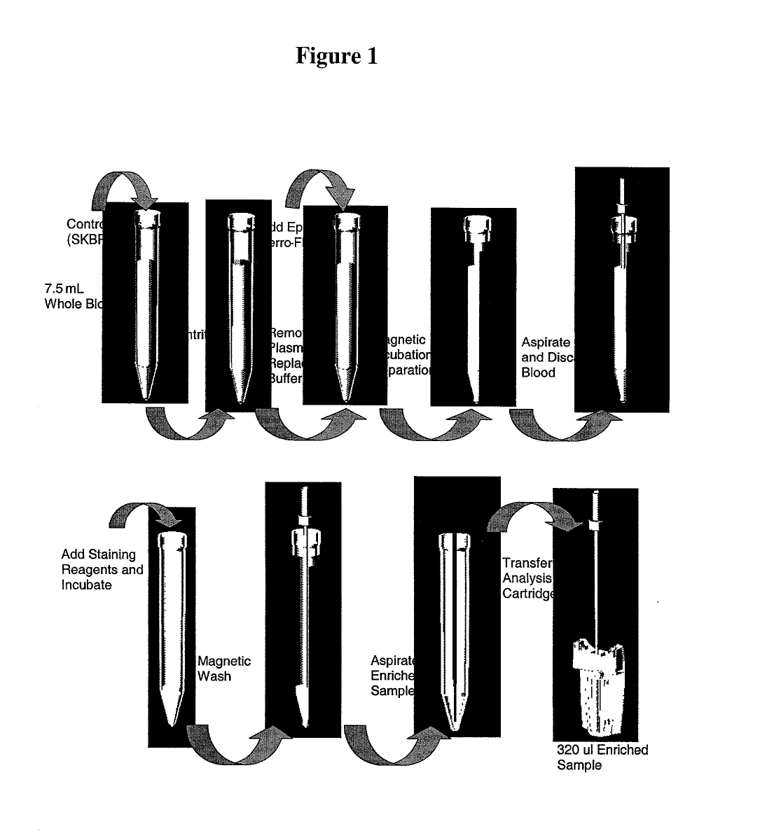

The steps involved in preparation of the sample for analysis are depicted in FIG. 1. To a 15 ml conical tube, 7.5 ml of blood, 6 ml of System Buffer and 100 μl (about 1000) of control cells are added and mixed. The sample is centrifuged at 800 g for 10 min and placed onto the system. The system locates the top of the packed red cell layer in the tube and a probe is introduced into the tube to aspirate the plasma without disturbing the buffy coat layer. The tube is taken from the system and 6 ml of AB buffer and 100 μl of CA-EpCAM ferrofluid are added and mixed. The tube is placed in the system and the sample tube is inserted and withdrawn from the magnetic separator under system control, thereby providing precise control over separation and removal times. While in the magnetic field the ferrofluids are moving laterally through the blood sample thereby increasing the labeling efficiency of potential target cells as well as moving the magnetically labeled CTC and unbound ferrofluid to the wall of the tube. After incubation and separation for 20 minutes, the probe is slowly lowered into the tube, aspirating and discarding the blood sample to waste. The tube is mechanically moved out of the separator and 3 ml of System Buffer is added to the tube. The collected cells and ferrofluid are resuspended by mixing the tube. The system lowers the tube into the magnet and aspirates uncollected material after 10 minutes. The tube is moved out of the magnet by the system and 200 μl of Immunoperm and 60 μl of staining reagents are added and mixed. After incubation for 15 minutes the excess staining reagents are aspirated and discarded by the system and the tube is moved out of the magnet. Ten minutes after 250 μl Cellfix is added, the system transfers the sample into the chamber. The volume of the chamber is approximately 320 μl and the system uses 100 μl of system buffer as a rinse to assure that residual cells in the tube are transferred into the chamber. The chamber is slightly overfilled to avoid air entrapment during capping with the plug seal. After each step in which a probe touches a sample, the probe is thoroughly washed by the system to eliminate any cell or reagent carryover.

CellSpotter® Analysis chamber

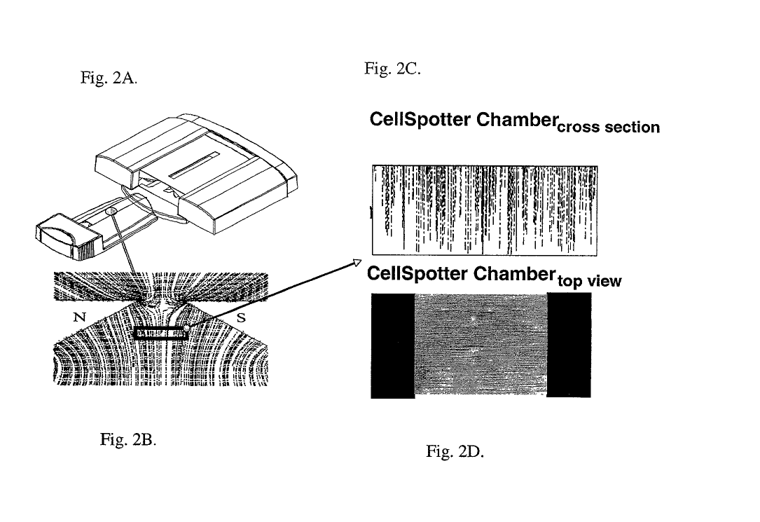

FIG. 2A shows the analysis chamber and the magnet yoke assembly that holds the chamber between the two magnets. To determine the optimal angle of the magnets and the optimal position of the chamber with respect to the two angular shaped magnets a computer program was written to simulate the movement of magnetically labeled cells in the chamber. The objective was to move all magnetically labeled cells to the upper surface of the chamber while preventing movement to the magnet poles. The distance from the chamber surface to the surface of the magnet must also be short enough to permit viewing through a microscope objective. FIG. 2B shows such a simulation, the chamber is outlined between the North (N) and South (S) pole of the magnets and the dashed lines indicate the trajectory of magnetically labeled cells. FIG. 2C shows a magnification of the trajectory within the chamber. All cells labeled with magnetic nanoparticles move vertically within the chamber. FIG. 2D shows the top view of the CellSpotter® chamber from an experiment in which cells and magnetic nanoparticles are introduced into the chamber. The horizontal lines distributed homogeneously over the surface of the chamber represent the magnetic nanoparticles that align along the magnetic field lines.

Data acquisition

The surface of the chamber is 80.2 mm² and has to be scanned completely for any objects staining with DAPI, DiOC16, CK-PE and CD45-APC. The combination of the objective and digital camera results in a pixel size of 0.45 (0.67×0.67) μm² and an image size of 858×686 μm. To cover the complete chamber surface, the CellSpotter® system acquires 4 rows of 35 images for each of the 4 filters resulting in 140 frames and 560 images per chamber. When the testing of a sample commences, the CellSpotter® acquisition program automatically determines the region over which the images are to be acquired, the number of images to acquire, the position of each image and the microscope focus to use at each position. The image acquisition region is determined by moving the X and Y stages to 5 positions that should have an edge of the chamber visible in the image. The software determines the edge location, draws a line on the image display where it has found the line, and gives the operator the ability to approve or override the selected edge location. Two measurements are made on one of the long edges of the chamber to also determine the angular offset of the chamber relative to the X-axis of the stage. All images need to be in focus over the whole imaged area. The depth of focus of the microscope is less than 10 μm. While the chamber surface is planar to within 10 μm, mechanical tolerances within the microscope stage, magnetic yoke and chamber may cause an angular skew in the Z-axis. Due to time restraints it is not feasible to have the software iteratively find the focus at each of the 140 imaging locations on a sample chamber. An algorithm was developed whereby the software performs an iterative determination of the focus at 5 locations on the chamber using the light emitted by the nucleic acid stain of the cells. The software then fits the empirical focus data to obtain a second order polynomial fit, which is used to determine the focus or Z-adjustment at every image location on the sample chamber. This iterative focusing procedure has the unique feature to perform the focusing algorithm only on cells in a user configurable size range and ignores non-cellular objects in the sample. In the event no suitable particles are found, the system will move to alternate focus points in the sample. All the images from a sample are logged into a directory that is unique to the specific sample identification.

Data analysis

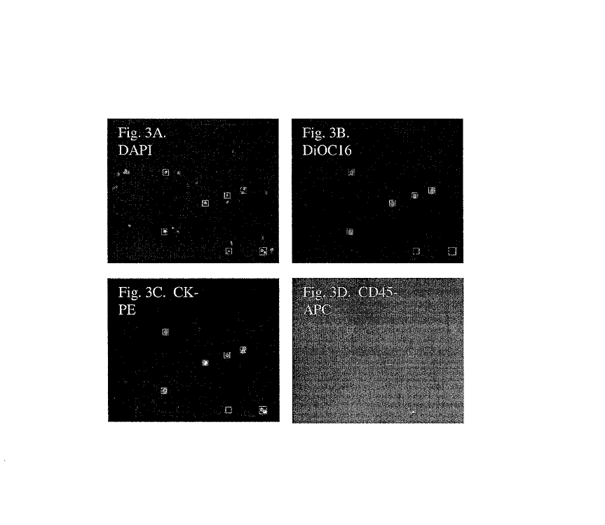

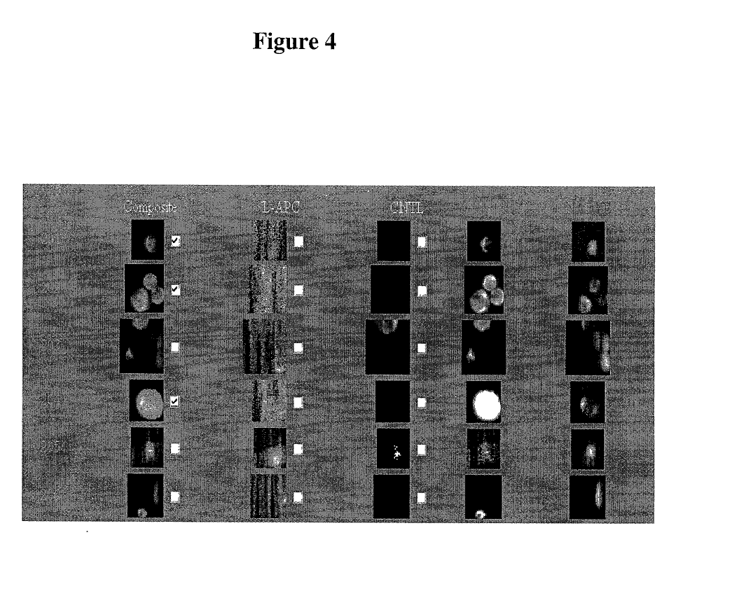

FIGS. 3A-3D shows the images of DAPI (Panel 3A), DiOC16 (Panel 3B), CK-PE (Panel 3C) and CD45-APC (Panel 3D) of one of the 140 frames obtained after processing a 7.5 ml blood sample from a patient with breast cancer. In the DAPI image multiple cell nuclei can be observed. A rectangular box is drawn around 7 nuclei. The corresponding DiOC16 image shows the same 7 rectangular boxes. In 5 of these boxes, round fluorescent objects are present typical for the control cells added to the blood prior to sample processing. The control cells also stain brightly for CK-PE as illustrated by the bright staining of the cells in the same 5 boxes shown in the CK-PE image. Two of the 7 boxes not staining in the DiOC16 (control) image stained in the CK-PE image. One of the boxes shows two nuclei and bright CK-PE staining corresponding to two cells. The CD45-APC image showed no staining in the box confirming that the box contained two cells of epithelial cell origin. The other box showed dim CK-PE staining and bright CD45-APC staining excluding this event as an epithelial cell. An algorithm is applied on all of the images acquired from a sample to search for locations that stain for DAPI, DiOC16 and CK-PE. If the staining area is consistent with that of a control cell (DiOC16+, CK−, PE+), the software assigns this location (box) to a control cell. The data analysis software tabulates the number of control cells found in a sample. If the staining area is consistent with that of a potential tumor cell (DAPI+, DiOC16−, CK-PE+), the software stores the location of these areas in the database. The software displays thumbnails of each of the boxes for each of the parameters in rows. From left to right these thumbnails represent the nuclear (DAPI), cytoplasmic cytokeratin (CK-PE), control cell (DiOC16) and surface CD45 (CD45-APC) staining. The composite images shown at the left show a false color overlay of the nuclear (DAPI) and cytoplasmic (CK-PE) staining. Check boxes beside the composite image and CD45-APC box allow the user to confirm that the images represented in the row are consistent with tumor cells or stain with the leukocyte marker CD45. The software tabulates the checked boxes for each sample and the information is stored in the database. Thumbnails of six staining areas that show staining characteristics consistent with CTC from a breast cancer patient sample are shown in FIG. 4. The images of three of the six staining areas clearly display features of CTC as visualized by the presence of a clear nucleus, cytoplasmic cytokeratin staining and absence of DiOC16 and CD45 staining. Differences in the appearance of the tumor cells were noted: the cell in row 201 is relatively small, a cluster of 3 tumor cells is present in row 202 and one very large cell is shown in row 204. In row 203, a control cell is shown in the top of the box. The area is displayed because of the CK-PE positive event below the control cell. The nuclei shown, however, belong to leukocytes and do not coincide with the CK-PE staining. Row 205 shows debris that stains positively in all four filters and in row 206, the CK-PE positive event does not coincide with the DAPI staining.

System Performance

To compare manual versus system sample preparation, 7.5 ml aliquots of blood were spiked with control cells and processed with both methods. In six experiments, the average tumor cell recovery was 68% for manual and 94% for the system sample preparation with a coefficient of variation of 10% and 7% respectively. The linearity of the system was tested by spiking control cells (n=1000) and 0, 50, 100, 150 and 200 cells of the tumor cell line SKBR-3 in 7.5 ml aliquots of blood obtained from 5 normal blood donors. The average recovery of control cells in these 25 experiments was 81% with a coefficient of variation of 7.8% demonstrated that the samples were properly processed. The correlation between the number of cells spiked and the number of cells recovered was r²=0.99 with a slope of 0.84 and an intercept of 3.2 indicating a tumor cell recovery of 84% that is independent from the level of tumor cells spiked. Spiking 0 and 10 tumor cells into 7.5 ml aliquots of blood from 10 donors was done to test the sensitivity of the system. The average control cell recovery in the 20 experiments was 85% with a coefficient of variation of 7.7%. The certainty of the actual spike numbers decreases with the number of cells spiked. The experiments were performed on two different days: one day the coefficient of variation in spiking 10 cells was 25% and on day two 15%. In the unspiked samples no tumor cells were found after processing. In contrast tumor cells were detected in all spiked blood samples ranging from 6 to 15 tumor cells (mean 10.5 cells CV 32%). The data clearly demonstrate that the sensitivity of the system is limited only by the blood volume processed.

To characterize the performance of the sample preparation and analysis system among different sites and operators, six systems were placed at different sites. Blood samples from 99 healthy donors were processed in duplicate at these sites. The average recovery of the control cells across the six sites was 77.1% with a coefficient of variation of 9.7%. As expected, the reproducibility between duplicate samples is better with a coefficient of variation of 4.9%. The number of events that were classified by the software as potential tumor cell candidates varied between 10 and 304 events with a mean of 55. Review of the candidate events showed that in 28 of the 192 blood samples (14.6%), one cell was found that classified as CTC, in 9 samples 2 cells (4.7%), in 3 samples 3 cells (1.6%) and in 1 sample 13 (0.5%) CTC were found. Table III shows the results of these experiments for each site. In 22 patients treated for metastatic breast cancer, 16 to 703 (mean 116) candidate CTC were found. Review of the candidate events showed that in 13 of the 22 blood samples, fewer than 3 cells were found that classified as CTC, in 4 samples 3-10 CTC were found and in 5 samples more than 10 CTC were found.

Circulating tumor cells can be detected in the blood of patients with carcinomas, albeit at extremely low frequencies. To investigate the potential use of CTC in the management of cancer patients a system that can accurately and reliably enumerate and characterize CTC is needed to perform controlled clinical studies. The number of CTC may represent tumor burden and changes in the CTC numbers could offer a means to evaluate the effectiveness of a given treatment. Analysis of the CTC for the presence or absence of therapeutic targets could be used to guide treatment. Detection of CTC in purportedly healthy individuals represents an advance in the early detection of cancer. If such early detection is possible a non-invasive, “whole body” biopsy of a solid tumor can be performed by a blood test.

To this end a semi-automated system was developed that immunomagnetically separates epithelial cells from 7.5 ml of blood, concurrently reduces the specimen volume and labels the cells immunofluorescently. The system produces a 320 μl liquid sample that is transferred to an analysis chamber and a magnetic device that causes all magnetically labeled cells in the sample to be pulled to the upper inside surface of the chamber for analysis. Four-color fluorescent analysis is performed on the sample by the CellSpotter® system that enumerates internal control cells and identifies objects that potentially classify as tumor cells by their positive staining of the nucleus, cytoplasmic cytokeratin and their lack of cell surface staining for CD45. Thumbnails of all objects that potentially classify as tumor cells are presented in the user interface from which the user can make the ultimate judgment.

Sample preparation performed by the system provides advantages when compared to the manual preparation of blood samples as demonstrated by a higher recovery and better reproducibility of enumerated tumor cells. Data from spiking experiments demonstrated an excellent linearity and sensitivity of the system. To demonstrate reproducibility of the system duplicate blood samples from 99 normal donors were processed at six different sites. Data across all sites demonstrated a level of reproducibility as assessed by recovery of internal control cells. The average recovery of the internal control was 77.1% with a coefficient of variation that varied between 3.2% and 11.8% (mean 9.7%). The sensitivity of the system was determined by the ability to detect CTC in patient samples and the specificity by identification of CTC in blood of normal donors. The analysis software identified between 10 and 304 (mean 55) candidate events in 192 normal blood samples and review of these candidates showed an average of 0.4 events that classified as CTC. In 22 patients treated for metastatic breast cancer 16 to 703 (mean 116) candidate CTC were found and 0-59 (mean 7) classified as CTC. In the blood of 15 out of 22 patients the number of CTC exceeded the upper limit of the 99% confidence interval for the average CTC count in normal individuals (mean +2.6*SE=0.56). In one normal blood sample, 13 events were classified as CTC by the operator, but review of the data revealed that this could be attributed to internal control cells that were weakly stained with the DiOC16. To avoid potential false positive results due to misclassification of control cells, addition of the internal control cells will only be done to demonstrate system and operator proficiency before running actual patient samples. The consistent and reproducible results between systems and operators in sample preparation and CTC analysis offers the opportunity to perform controlled clinical studies for elucidating the role of CTC levels in management of patients with carcinomas.

TABLE III
Analysis of 192 blood samples of 99 healthy controls

| | | Mean CC | | CV(%) | | Candidate events | | | CTC |
|---|---|---|---|---|---|---|---|---|---|
| Site | n | Recovery(%) | CV(%) | Across | Duplmin | max | mean | max | mean |
| 1 | 45 | 75.2 | 11.8 | 7.5 | 19 | 158 | 61 | 2 | 0.3 |
| 2 | 48 | 80.2 | 6.4 | 6.9 | 10 | 145 | 40 | 3 | 0.6 |
| 3 | 12 | 85.1 | 3.2 | 2.6 | 43 | 304 | 119 | 0 | 0.0 |
| 4 | 18 | 76.7 | 11.2 | 4.2 | 23 | 115 | 60 | 1 | 0.1 |
| 5 | 16 | 77.7 | 5.5 | 3.2 | 17 | 182 | 60 | 3 | 0.4 |
| 6 | 53 | 74.0 | 9.2 | 5.4 | 13 | 134 | 46 | 13 | 0.3 |
| all | 192 | 77.1 | 9.7 | 4.9 | 10 | 304 | 55 | 13 | 0.4 |

n = number of events
CC = control cells

### EXAMPLE 3

Enumeration of circulating epithelial cells in patients treated for metastatic Breast cancer

The following methods are provided to facilitate the practice of the following examples.

Patients. With informed consent, 8-20 ml blood samples were obtained from controls and patients with carcinoma of the breast, prostate and colon. Blood was drawn from some of these patients at several time points over a period of one year. The blood samples were drawn into Vacutainer tubes (Becton-Dickinson) containing EDTA as anticoagulant. The samples were kept at room temperature and processed within 24 hours after collection. The circulating epithelial cells were enumerated in peripheral blood samples from breast, prostate and colon cancer patients and in normal controls with no evidence of malignant disease. Date of diagnosis, therapeutic interventions and clinical status were retrieved from the patient's charts. The institutional review board of the collaborating institutions approved the protocol.

Sample preparation. Monoclonal antibodies specific for epithelial cell adhesion molecule (EPCAM) are broadly reactive with tissue of epithelial cell origin (Stahel RA, et al. Int J Cancer Suppl. 8:6-26 (1994); Momburg F, et al. Cancer research. 47:2883-2891 (1987); Gaffey MJ, et al. Am J Surg Path. 16:593-599 (1992)). The GA73.3 or MJ37 EpCAM antibodies recognizing two different epitopes on EpCAM (kindly provided by D Herlyn (Herlyn D, et al. J Immunol Methods. 73:157-167 (1984)) Wistar Institute, Philadelphia, Pa. and MJ Mattes (De Leij L, et al. Int J Cancer Suppl. 8:60-63 (1993)) Center for Molecular Medicine and Immunology, N.J.) were coupled to magnetic nanoparticles (ferrofluids) (Liberti PA & Piccoli SP, U.S. Pat. No. 5,512,332 (1996), Immunicon, Huntingdon Valley, Pa.). Blood was incubated with the anti-EpCAM conjugated ferrofluid for 15 minutes in disposable tubes with an internal diameter of 13 mm. The tubes were placed into a separator composed of four opposing magnets for 10 minutes (QMS13, Immunicon, Huntingdon Valley, Pa.). After separation, the blood was aspirated and discarded. The tube was taken out of the magnetic separator and the collected fraction was resuspended from the walls of the vessel with 2 ml of FACS permeabilization solution (BDIS, San Jose, Calif.) and placed in the magnetic separator for 5 minutes. The solution was aspirated and discarded and the cells were resuspended in 150 μl of cell buffer (PBS, 1% BSA, 50 mM EDTA, 0.1% sodium azide) to which phycoerythrin (PE) conjugated anti-cytokeratin (CAM5.2 Monoclonal antibody) and Peridinin Chlorophyll Protein (PerCP)-labeled CD45 were added at saturating conditions. After incubation for 15 minutes, 2 ml of cell buffer was added and the cell suspension was magnetically separated for 5 minutes. After discarding the non-separated suspension, the collected cells were resuspended in 0.5 ml of the buffer to which the nucleic acid dye used in the Procount system from BDIS, San Jose, Calif., was added according to manufacturer's instructions. In some cases in which the EpCAM antibody MJ37 was used on the ferrofluid, GA73.3 PE was used to identify the selected epithelial cells. In these cases no permeabilization of the cells is required. Reagents for flowcytometry were kindly provided by BDIS, San Jose, Calif.

An exemplary method for determining the tissue source of circulating epithelial cells employs cytochemical and immunological identification techniques. Primary monoclonal antibodies recognizing cytokeratins 5, 6, 8, 18 (CK, 5D3, LP34, Novocastra), MUC-1 glycoprotein (MUC-1, Ma695 Novocastra) or prostate specific antigen (PSMA), clone J591 obtained from Dr. Neil Bander (Cornell University, Ithaca, N.Y.) was added to the slides after blocking non-specific binding sites with 5% BSA for 30 minutes. The samples were incubated for 20 minutes at room temperature, washed twice in PBS for 5 minutes and then exposed to secondary rabbit anti-mouse Ig (Z0259, Dako Corp., Carpenteria, Calif.) for another 20 minutes. After two more washes, the samples were incubated with alkaline-phosphatase-anti-alkaline phosphatase (APAAP) rabbit Ig complexes for 15 minutes. Finally, the enzyme-substrate (New Fuchsin, Dako Corp. Calif.) was added resulting in the development of red precipitates. The nucleus was counterstained with hemotoxylin. The data were recorded using a Kodak digital camera attached to a light microscope. Data could be stored on CD for later reference. Sample analysis. 85% of the samples were analyzed on a FACSCalibur flowcytometer (BDIS, San Jose, CA). The data were acquired in listmode using a threshold on the fluorescence of the nucleic acid dye. Multiparameter data analysis was performed using Paint-A-GatePro (BDIS, San Jose, Calif.). Analysis criteria included size defined by forward light scatter, granularity defined by orthogonal light scatter, positive staining with the PE labeled cytokeratin monoclonal antibody and no staining with the PerCP labeled CD45 monoclonal antibody. For each sample, the number of events present in the region typical for epithelial cells was normalized to 10 ml of blood.

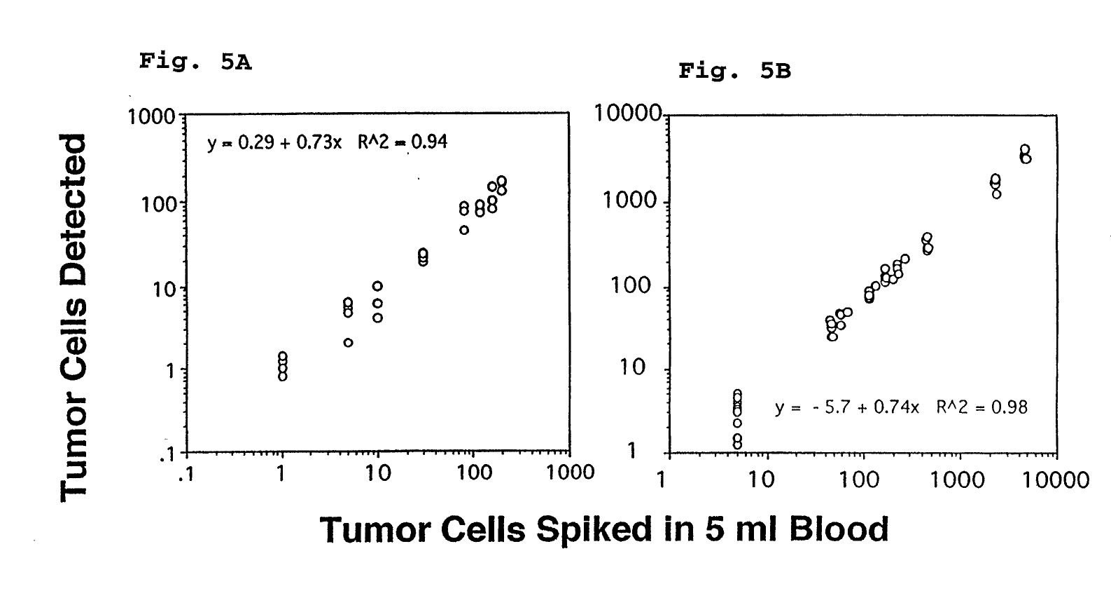

The results obtained when tumor cells spiked into whole blood are isolated using the assay methods of the present invention are shown in FIGS. 5A and 5B. Panel 5A shows analysis by microscopy and panel 5B shows analysis results obtained using flowcytometry. FIGS. 6A-6C show three examples of the flowcytometric analysis of 10 ml blood samples obtained from one patient with metastatic breast carcinoma at three time points, and includes the correlative display of the anti-leukocyte versus anti-epithelial cell antibodies of the flowcytometric analysis. In FIG. 6, Panel A, 14 events are detected and are present in the location typical for epithelial cells. In Panel 6B, 108 epithelial cells are detected and in Panel 6C, 1036 epithelial cells are detected.

The number of events passing the threshold set on the nucleic acid dye in the analysis of the 10-ml blood sample varied between 5,000 and 50,000 events. These events consist of cellular debris and leukocytes. In analyzing the blood of 32 controls, the number of events present in the region typical for epithelial cells ranged from 0-4/10 ml of blood (mean=1.0, SD=1.2).

Eight breast cancer patients had active metastatic disease during the period of study. In these patients, the number of epithelial cells in 10 ml of blood varied within the range of 0 to 1036. The activity of the disease was assessed by subjective criteria, i.e. bone pain, dyspnea etc. and objective criteria, X-rays, bone scans, CT scan, MRI and lymph node size. Patients were classified in categories 0 through 4,as set out in Table VI.

TABLE IV
Classification of patients according to clinical activity
of the disease after surgical intervention

| Category | Criteria |
|---|---|
| 0 | No evidence of disease at any time point after |
| | surgical intervention |
| 1 | Evidence of disease at one time point |
| | after surgical intervention |
| 2 | Evidence of disease under control |
| 3 | Active progressive disease |
| 4 | Life threatening disease |

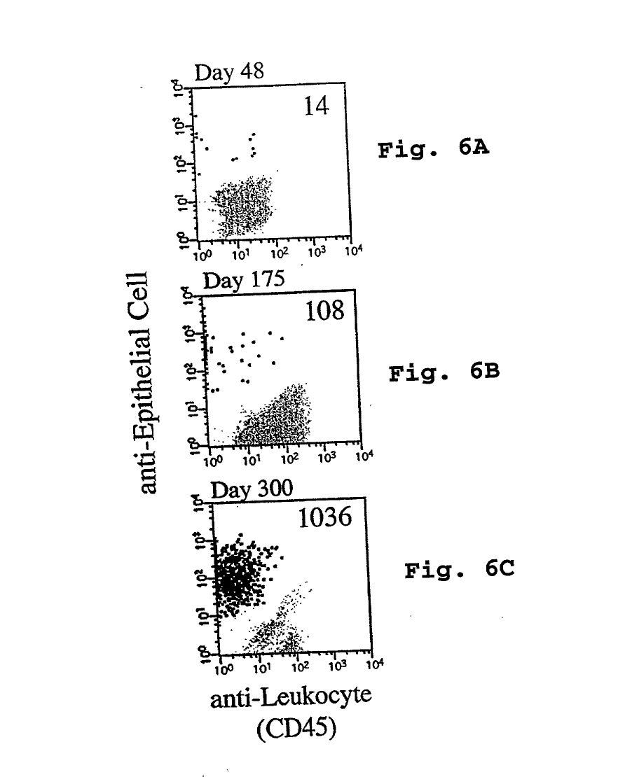

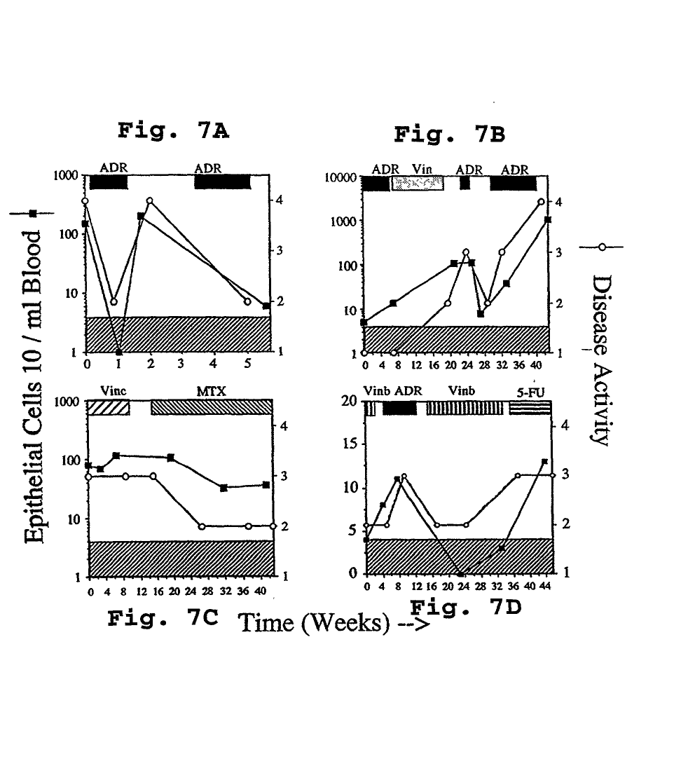

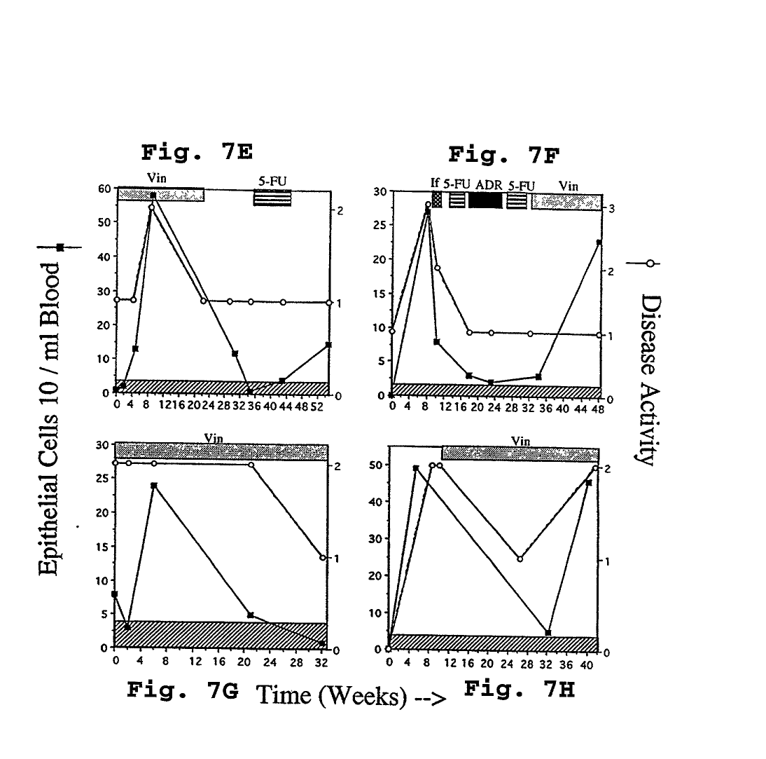

The dynamics of epithelial cell counts in the blood of 8 patients with metastatic disease are presented in FIG. 7. The shaded area in the plots indicates the range at which positive events were detected in the controls. The plots also indicate when chemotherapy was administered. FIG. 7, panel A shows a patient with life threatening disease and 200 epithelial cells/10 ml of blood at the time she entered the study. High dose adriamycine reduced the number within the normal range, but it rose again after adriamycine was discontinued. After a second course of adriamycine, the number of epithelial cells dropped significantly, but was still above the normal range. FIG. 7, Panel B shows the course of one patient over a period of 43 weeks. The patient was asymptomatic at the start of the study but was known to have bone metastasis in the past. Epithelial cells were detected above normal levels and steadily increased during the period studied. A brief decline in the number of epithelial cells was found after a course of high dose adriamycine was administered. The activity of disease in this patient clearly increased during this period. In FIG. 7, Panels C and D, two patients are shown with less disease activity. In these patients, the changes in the number of epithelial cells over time also reflected the changes in the activity of the disease. In the patients shown in Panels 7E and 7F, the number of peripheral blood epithelial cells increased at the last time point studied while the patients still were without symptoms.

In the case shown in Panel 7G, no epithelial cells were detected at the first time point studied which was three years after breast cancer surgery (T2N1M0). Four weeks later, 50 epithelial cells in 10 ml of blood were detected by flowcytometry. The patient at this time had no clinical signs of disease recurrence. An additional blood sample was analyzed to obtain morphological confirmation that the cells detected by flowcytometry had features consistent with those of malignant cells.

FIG. 8A shows two cells with a large nuclear to cytoplasmic ratio and which positively stain with Cytokeratin, both features being consistent with tumor cells of epithelial cell origin. Four weeks after this finding, the patient had an axillary lymph node biopsy. Cells obtained from the biopsy proved to be of malignant origin. Although an X-ray at this time did not show signs of pulmonary metastasis, a CT scan performed two weeks later showed evidence of pulmonary metastasis. The patient had no symptoms from the pulmonary metastasis. The patient reacted well to Vinorelbine as measured by the disappearance of the axillary lymphnode involvement. The peripheral blood epithelial cell number dropped to levels just above the normal range. Twenty-eight weeks after initiation of the treatment, the peripheral blood epithelial cell number increased and by physical examination, the axillary node increased in size. The number of peripheral blood epithelial cells in these 8 patients with metastatic disease of carcinoma of the breast clearly reflected the activity of the disease and the response to treatment or the lack thereof during the time period studied.

The experiments described above were performed using colloidal magnetic nanoparticles. In this example, the efficiency of larger size magnetic beads for the selection of tumor cells present at a low frequency in blood was also evaluated to determine whether micron size beads could also be used to select tumor cells. However, as described above, nanometer size magnetic particles are considered preferable for this application.

As mentioned previously, disadvantages are encountered with the use of larger size beads. These are:

(i) the beads are too large to diffuse thus collisions of the beads with target cells present at a low frequency requires mixing,

(ii) the beads settle very fast, furthering the need for continuous mixing, and

(iii) large size beads cluster around cells and obscure analysis.

Accordingly the large size beads need to be removed from the cell surface prior to visualization or analysis. In accordance with the present invention, it has been found that the efficiency of cell selection with larger beads can be improved by increasing the concentration of beads and increasing the incubation time with continuous mixing to facilitate binding to rare target cells. In this example, 2.8 μm Dynal anti-epithelial cell beads (Dynal, N.Y.) were used to test the efficiency of tumor cell selection from blood in a model study under optimum conditions for large beads. These beads are conjugated with a monoclonal antibody specific for epithelial tumor cells. A known number of tumor cells (cancer cell line) were spiked into normal blood to determine the recovery after selection with beads. The tumor cells were pre-labeled with a fluorescent dye to differentiate them from blood cells during detection. The protocol was followed as recommended by the manufacturer.

Whole blood (5 ml) was added to a 15 ml polystyrene centrifuge tube followed by the addition of 20±3 fluorescently labeled SKBR-3 (breast cancer cell line) cells. SKBR-3 cells were pre-stained with a nucleic acid staining dye (Hoechst 33342) to allow detection after the selection by beads. The blood was diluted with 5 ml of Dulbecco's PBS containing 5 mM EDTA and mixed with the diluted blood for 15 minutes at 4° C. on a rocker. 10Opl of Dynal anti-epithelial cell beads containing 50×106 beads were added to the blood sample and incubated for 30 minutes at 4° C. with mixing on a rocker. Note that the number of beads used were similar to total white blood cells i.e. one bead per white cell. The magnetically labeled cells were separated by placing the sample tube into Dynal MPC magnetic separator for 6 minutes.

After aspirating the supernatant, the collected cells were resuspended in 3 ml of Dulbecco's PBS containing 0.1% BSA. The sample tube was placed back into Dynal's MPC for 6 minutes to remove any carry-over blood 5 cells. The magnetically bound cells were resuspended in 200 μl of Dulbecco's PBS containing 0.1% BSA after aspiration of the supernatant.

The final sample, containing selected tumor cells, non-specifically bound blood cells and excess free magnetic beads, was spotted onto an immunofluorescent slide to detect tumor cells. The 200 μl sample was spotted into 10 different wells to disperse free magnetic beads. The fluorescently stained tumor cells present in each well were counted using a fluorescent microscope. The results are shown in the Table V:

TABLE V

| Experiment No. | Tumor cells recovered | % Recovery |
|---|---|---|
| 1 | 16 | 80 |
| 2 | 17 | 85 |
| 3 | 10 | 50 |
| 4 | 11 | 54 |

The results show that, on average, 67% of the spiked tumor cells were recovered from blood by Dynal magnetic beads. This suggests that tumor cells present in blood can be selected efficiently with larger size magnetic beads under optimum conditions. In this example, however, only the selection of tumor cells from blood was evaluated without performing any analysis. Further the efficiency of recovery could be determined because cells were pre-labeled with a strong fluorescent dye. The final sample (200 μl ) contained 50×10⁶ beads in addition to selected tumor cells (10-17) and non-specifically bound leukocytes. The size of the beads (2.8 μm) is similar to that of certain blood cells and occupied most of the surface area on the slide. Therefore, to obtain recovery data, the sample had to be spotted onto several wells in order to sufficiently disperse free magnetic beads so as to allow for detection of recovered tumor cells.

There were also many beads on cell surfaces that preclude viewing and staining of selected tumor cells for further analysis. In this example, tumor cells were pre-stained with a fluorescent nucleic acid dye and further staining was not necessary for detection. However it is often desirable to identify the tissue of origin of the magnetic bead-bound cells. Such identification is performed using labeled antibodies to detect and characterize tumor cells present in clinical samples. Accordingly, beads have to be removed from cell surfaces and separated from the sample following target cell selection, i.e. before analysis. This is not the case with magnetic nanoparticles because their size does not interfere with cell analysis.

In summary, this example shows that large magnetic beads may also be utilized in the methods disclosed herein for the efficient isolation of circulating tumor cells.

There are several methods available to release beads from cell surfaces that do not significantly damage isolated cells. One method is to displace antibody from the cell surface by adding an excess specific competing reagent in excess that has higher affinity for the involved antigen or antibody. This type of mechanism is used to release beads from CD34 selected cells in clinical applications using a peptide (Baxter Isolex 300). The peptide competes with CD34 antigen for binding to antibody on beads and releases the antibody-bead complex from cells. Another method employs a reversible chemical linker between beads and antibodies.

The chemical linker can be inserted during the conjugation of antibodies to magnetic beads. The chemical link can be cleaved under appropriate conditions to release beads from antibodies. One of the methods currently in use employs a nucleic acid linker to link antibodies to magnetic beads. The nucleic acid linker is a polynucleotide and can be hydrolyzed specifically using DNAse enzyme. Following hydrolysis of the nucleotide bonds present in the nucleic acid linker, the beads are released from the antibodies that remain bound to cells. The released beads can be removed from cell suspension by magnetic separation. The cells that are freed from beads can be used for further analysis by microscopy or flow cytometry.

This example demonstrates that larger size magnetic beads can also be used to isolate tumor cells from blood, provided they are used in high enough concentration to label cells and are then released from cells before analysis.

### EXAMPLE 4

Enumeration of circulating epithelial cells in patients with no evidence of disease after surgery for carcinoma of the breast with curative intent

Peripheral blood of 37 patients between 1 and 20 years after surgery was examined for the presence of epithelial cells by flowcytometry. Up to 7 peripheral blood samples were taken over a one-year period from these patients. In Table VI, each of the patients is listed and sorted according to the TNM (tumor, node, and metastasis) stage at the time of surgery followed by the years after surgery. Table VI also shows whether or not the patient received treatment (either chemotherapy or hormonal therapy) during the period studied. In 3 of 6 patients with evidence of distant metastasis in the past, but in complete remission at the time of study, epithelial cells were found in the blood at a higher frequency than that found in the control group. Circulating epithelial cells were also found in 9 of 31 patients with no evidence of distant metastasis.

The low number of events present in the region typical for epithelial cells by flowcytometry in these 9 patients does not warrant identifying these events as tumor cells. Cytology obtained by placing the immunomagnetically selected cells on a slide greatly aids in the assessment of their identity as is illustrated in FIG. 8. FIG. 8, panel A, shows two cells staining positive for cytokeratin and obtained from a patient with no evidence of metastatic disease at the time the blood was drawn. Panel 8B shows a cell from a patient with metastatic disease in the past but in complete remission. In Panels 8C and 8D, two cells are shown isolated from the blood of patient 25 at time point 6. The cell shown in Panel 8C has features consistent with malignancy whereas the cell in Panel 8D has the appearance of a normal squamous epithelial cell.

TABLE VI
Number of epithelial cells identified by flowcytometry in
10 ml of peripheral blood of patients with no evidence of
disease after surgery for carcinoma of the breast with
curative intent and 32 controls.

| Patient | | | | | | | | | | |
|---|---|---|---|---|---|---|---|---|---|---|
| Number | TNM | Ys | Tx | 1 | 2 | 3 | 4 | 5 | 6 | 7 |
| 1 | T₃N₁M₁ | 9 | — | 2 | 29 | | | | | |
| 2 | T₃N₁M₁ | 16 | H | 0 | | | | | | |
| 3 | T₂N₁M₁ | 7 | CT | 10 | 7 | 5 | 6 | 4 | 8 | 7 |
| 4 | T₂N₁M₁ | 10 | CT | 1 | 0 | 0 | 1 | 2 | | |
| 5 | T₂N₁M₁ | 10 | H | 6 | | | | | | |
| 6 | T₂N₁M₀ | 20 | H | 12 | 2 | | | | | |
| 7 | T₃N₁M₀ | 1 | H | 0 | | | | | | |
| 8 | T₃N₁M₀ | 2 | CT | 0 | 0 | 0 | | | | |
| 9 | T₃N₁M₀ | 2 | CT | 0 | 0 | 1 | 0 | | | |
| 10 | T₃N₁M₀ | 3 | H | 3 | | | | | | |
| 11 | T₃N₁M₀ | 3 | H | 5 | 4 | 0 | 6 | | | |
| 12 | T₃N₁M₀ | 3 | H | 3 | 0 | | | | | |
| 13 | T₃N₁M₀ | 6 | CT | 6 | 0 | | | | | |
| 14 | T₃N₁M₀ | 6 | H | 1 | 1 | | | | | |
| 15 | T₃N₁M₀ | 7 | H | 1 | 3 | 3 | | | | |
| 16 | T₃N₁M₀ | 3 | H | 0 | | | | | | |
| 17 | T₂N₁M₀ | 17 | H | 4 | | | | | | |
| 18 | T₃N₀M₀ | 3 | — | 5 | | | | | | |
| 19 | T₃N₀M₀ | 5 | H | 1 | | | | | | |
| 20 | T₃N₀M₀ | 8 | H | 0 | 6 | 8 | | | | |
| 21 | T₂N₀M₀ | <1 | — | 0 | | | | | | |
| 22 | T₂N₀M₀ | <1 | H | 0 | | | | | | |
| 23 | T₂N₀M₀ | 1 | H | 0 | | | | | | |
| 24 | T₂N₀M₀ | 1 | — | 4 | | | | | | |
| 25 | T₂N₀M₀ | 2 | CT | 3 | 5 | 1 | 3 | 6 | 2 | |
| 26 | T₂N₀M₀ | 3 | CT | 2 | 6 | 3 | 1 | 1 | 0 | 5 |
| 27 | T₂N₀M₀ | 6 | H | 18 | | | | | | |
| 28 | T₂N₀M₀ | 6 | H | 2 | 1 | | | | | |
| 29 | T₂N₀M₀ | 7 | H | 8 | 4 | 2 | | | | |
| 30 | T₂N₀M₀ | 8 | H | 0 | 1 | | | | | |
| 31 | T₂N₀M₀ | 8 | H | 0 | 6 | 8 | | | | |
| 32 | T₂N₀M₀ | 11 | H | 2 | | | | | | |
| 33 | T₂N₀M₀ | 20 | H | 4 | | | | | | |
| 34 | T₁N₀M₀ | <1 | H | 0 | | | | | | |
| 35 | T₁N₀M₀ | 2 | H | 0 | | | | | | |
| 36 | T₁N₀M₀ | 17 | — | 0 | | | | | | |
| 37 | T_{?}N₀M₀ | 13 | H | 0 | | | | | | |

N3 controls

| | |
|---|---|
| 2 | min 0 |
| | max 4 |
| | mean 1.0 M ″ 2SD 3.5 |

TNM = Tumor, Node, Metastasis
Ys = years after primary surgery
Tx = therapy, Ct = chemotherapy, H = hormonal therapy, — = no therapy
1, 2, 3, 4, 5, 6, 7 = subsequent time point at which the number of epithelial cells was determined in years

### EXAMPLE 5

Enumeration of circulating epithelial cells in patients diagnosed with breast cancer before surgical intervention.

Table VII summarizes the results obtained following similar clinical trials in which 13 controls and 30 patients with breast cancer were assessed using the assay of the invention. In control individuals the number of epithelial cells in 20 ml of blood ranged from 0-5 (mean 1.5 S.D.=1.8). In contrast, there was an average of 15.9 S.D.=17.4 epithelial cells in the 20 ml blood samples of 14 patients with organ-confined carcinoma of the breast (patients classified as TₓNₒMₒ), 47.4 S.D.=52.3 in those with nodal involvement, and 122 S.D.=140 in those with distant metastases. The difference between the control group and patients with carcinoma of the breast, with or without metastasis, was highly-significant [P,0.001 by multi-parameter analysis (Kruskal-Wallis)]. The difference between the organ-confined and the distant metastatic group was 0.009(t test). The number of epithelial cells in patients with organ-confined breast cancer was above the cut-off point (mean value plus 3 SD in the control group=6.9) in 12 of 14 cases. Moreover, no individual in the control group had more than 5 events classified as epithelial cells, and only 2 of the 14 patients with organ-confined breast cancer had <7 such events.

TABLE VII
Summary of clinical data

| | | | Spread to | |
|---|---|---|---|---|
| | Healthy | No detectable | lymphnodes | Distant |
| Number | Control | spread | only | metastasis |
| 1 | 0 | 0 | 7 | 20 |
| 2 | 0 | 4 | 8 | 20 |
| 3 | 0 | 7 | 14 | 20 |
| 4 | 0 | 8 | 93 | 23 |
| 5 | 0 | 8 | 115 | 54 |
| 6 | 0 | 8 | | 62 |
| 7 | 0 | 12 | | 99 |
| 8 | 2 | 13 | | 135 |
| 9 | 2 | 14 | | 152 |
| 10 | 4 | 16 | | 304 |
| 11 | 4 | 18 | | 456 |
| 12 | 5 | 19 | | |
| 13 | | 24 | | |
| 14 | | 72 | | |
| n | 12 | 14 | 5 | 11 |
| mean | 1.5 | 15.9 | 47.7 | 122.5 |

Flowcytometry was used to analyze the positive events obtained from 20 ml of blood from control individuals and from women with breast carcinoma. The numbers of epithelial cells in the blood of controls are statistically different by t test (P≦0.01) and by Kruskall-Wallis nonparametric analysis (P<0.001) from each of the three groups of the breast cancer patients. The data in this table were used to establish a preliminary cut-off value for positive samples. This value was determined by averaging the number of circulating epithelial cells in the normal controls (n=13) and then adding three times the SD. The average was 1.5 and the SD is 1.8. Cut-off: 1.5+5.4 =6.9. There is no statistical difference between male and female controls.

### EXAMPLE 6

MONITORING CTCs IN PATIENTS WITH METASTATIC CAP

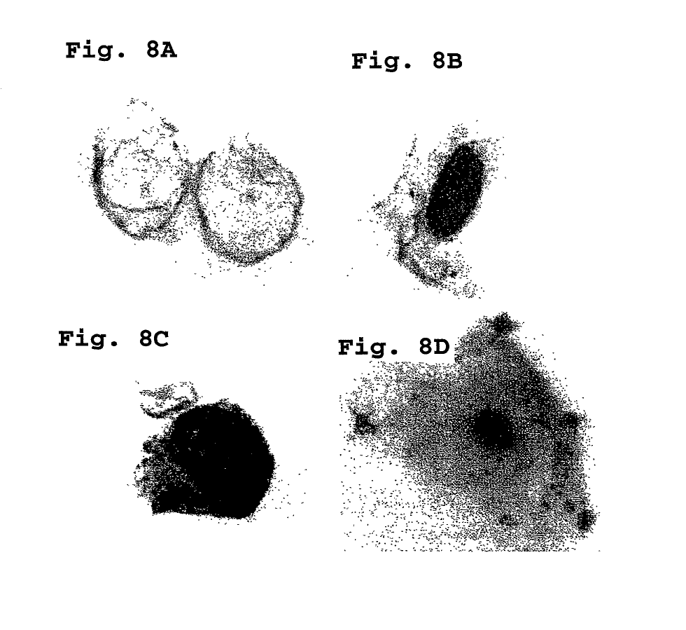

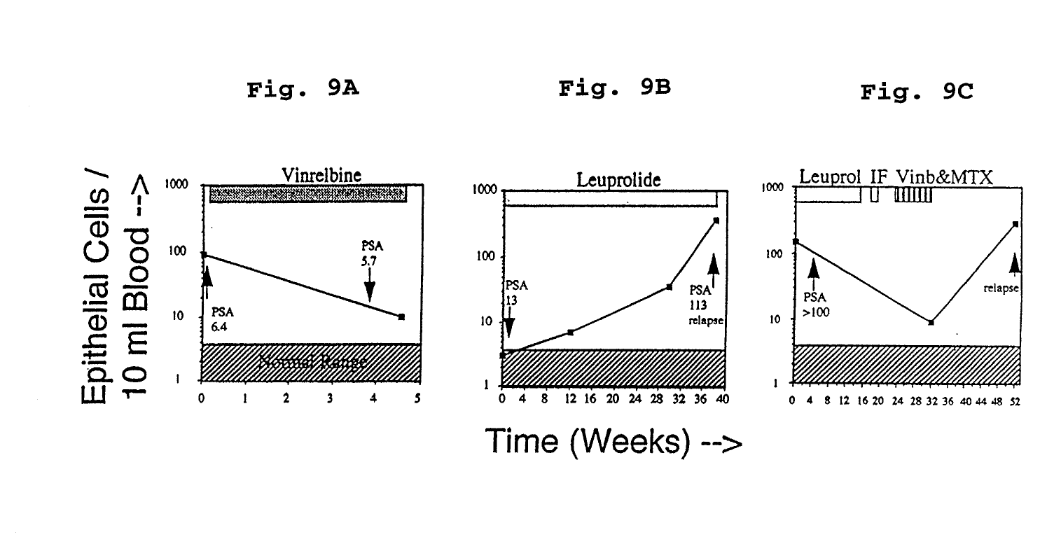

Three patients with metastatic disease of the prostate were assessed for the presence of circulating epithelial cells in their blood following chemotherapeutic treatment. The results are presented in FIG. 9. The data reveal that an increase in circulating epithelial cells in the blood is correlatable with disease activity.

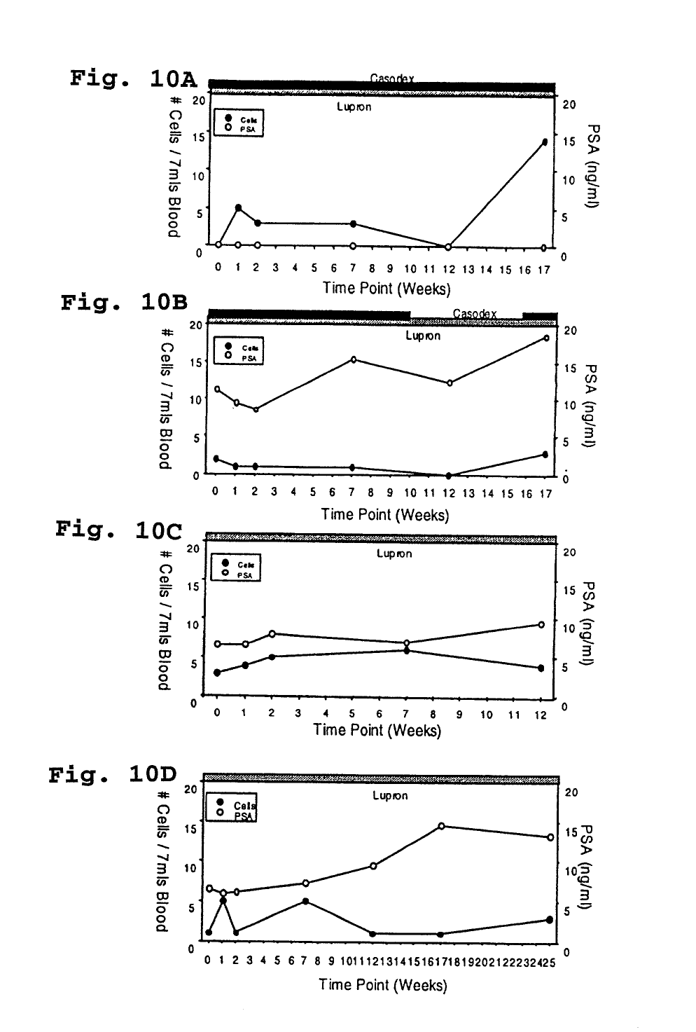

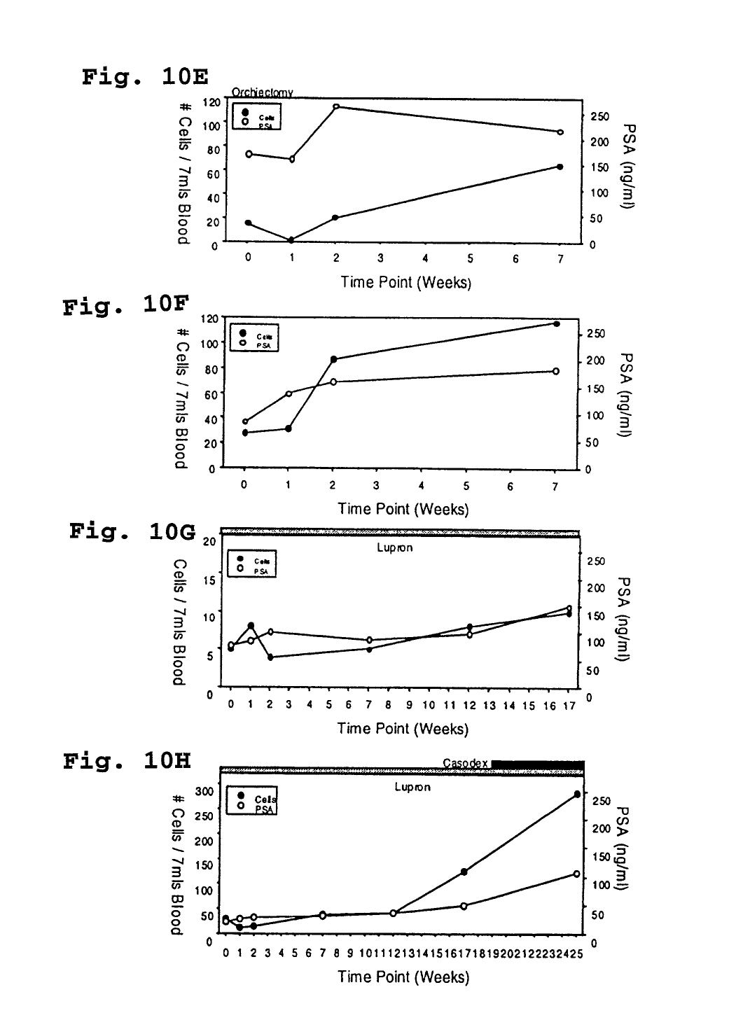

Ten patients were selected for serial testing of CTCs and PSA at intervals of 0, 1, 2, 7, 12, 17, and 25 weeks. Eight patients had hormone-refractory disease, one refused hormonal therapy, and one had hormone-sensitive disease. The patients° CTC and PSA levels are shown for each point in FIG. 10. The hormone-sensitive patient had PSA levels of less than 0.1 ng/mL during the study period, and the CTC numbers were comparable to the controls, except for the last point, at which 14 tumor cells were measured (FIG. 10A). The CTC count repeated 1 week later was 15 and confirmed the earlier observation. No signs or symptoms that suggested disease rogression were observed in this patient. Three patients had slow disease progression (FIGS. 10B-D). The mean CTC count in these patients was statistically different from the control group (3.0±3.0 tumor cells/7 mL, P=0.002, n =25). In 6 of the 25 samples, the CTC number was 5 or more per 7 mL. The CTC size was assessed by forward light scatter and in samples with 5 or more CTCs, 77%±15% of these cells were larger than 10 μm. Six patients had disease progression during the study period. The CTC and PSA values of four of these patients are shown in FIGS. 10E-H. The mean CTC counts were clearly different from the control group (range 1 to 283, n=22, mean 45±65 CTCs/7 mL) and statistically different from the patients with slowly progressing disease (P=0.008). In 19 of the 22 samples, the CTC count was 5 or more, and 51%±17% of the CTCs were larger than 10 μm. In this group of patients, the increase in CTCs paralleled the increase in the PSA level.

Two patients receiving estramustine and taxane-based chemotherapy had a pronounced difference in the CTC count compared with the control group and the patients who had slow disease progression (range 14 to 218, mean 104±68 CTCs/7 mL). Only 32%±13% of CTCs in these patients had sizes of 10 AM greater. A comparison of the CTC size among the three patient groups by t test showed significant differences between groups 1 and 2 (P=0.004), groups 1 and 3 (P=0.0001), and groups 2 and 3 (P=0.0008). The CTC and PSA values and the administration of chemotherapy are shown in FIG. 11 for both patients. Fluctuations in the CTCs concurred with the administration of chemotherapy. The relative changes in the CTCs were more pronounced than those of PSA, and the CTC count paralleled the PSA level in both graphs. However, the actual correlation was poor (FIG. 11A, R=0.17 and FIG. 11B, R=0.46).

The method of the present invention can quantify CTCs and was used to assess the CTC changes during HRPC progression. In vitro PC3 cell spiking experiments demonstrated a strong linear correlation (R² =0.99) and an excellent recovery rate (74%±9%). The detection limit of 0.8±1.2 cell in 7.5 nm of blood was determined by analyzing the blood of 22 normal male donors.

One gram of tumor sheds approximately 106 cells into the blood. Our observation that CTCs can be found in localized CAP is in agreement with previous examples assessing localized breast cancer. Ten patients with metastatic CAP were selected to undergo serial testing for CTC load and serum PSA level during 6 months. The patients were tested weekly for three courses and then approximately every 5 weeks for 6 months. Four men with either early hormone resistant prostate cancer (HRPC) (n=3) or hormone-sensitive disease (n=1) were evaluated. Their CTC counts were low (3.0±3) but significantly different than the control group (P<0.002). Overall, the CTC count trend did not rise dramatically and concurred with PSA pattern, with the exception of the patient with hormone sensitive disease (FIG. 10A). In this case, the CTC count rose precipitously at week 17 to 14 cells/7 nm blood and was confirmed 1 week later, suggesting that it was a genuine biologic event. The serum PSA level remained undetectable. Whether the rapid increase in the CTC level will precede biochemical PSA failure remains to be determined.

Four other men had rapidly progressive metastatic disease or HRPC. Their CTC counts were significantly higher (FIG. 10E-H) than the early HRPC group. The serum PSA and CTC values seemed to correlate. However, their calculated correlations varied (FIG. 10A, R=0.42; FIG. 10B, R=0.67; FIG. 10C, R=0.65, and FIG. 10D, R=0.98). The more disparate correlation (R=0.42) occurred in the patient shown in FIG. 10E, who died of uremic coma from metastatic CAP, which caused obstructive uropathy because of bulky retroperitoneal disease. We postulate that metabolic derangement may have caused a precipitous transient drop in the CTC count at week 1. The strongest correlation occurred in the patient shown in FIG. 10H (R=0.98). This patient had a rising PSA level that closely mirrored a dramatic elevation in the CTC count. He went on to develop symptomatic progression but declined chemotherapy.

Two of 10 patients underwent chemotherapy that had an impact on both the serum PSA level and the CTC counts in a similar fashion (FIG. 11). These patients maintained CTC numbers that ranged between 14 and 218 tumor cells/7 mL. In both cases, we observed a substantial decrease in the CTC count 1 week after the first Taxotere dose, and this paralleled a drop in the PSA level. Both patients exhibited a significant rise in the CTC and PSA levels, despite continued doses of Taxotere. The patient shown in FIG. 11B was then switched to Taxol and the patient shown in FIG. 11A continued with Taxotere. They both showed a drop in the CTC count between weeks 7 and 12 that paradoxically was associated with either a rise or an unchanged PSA level. Eight weeks after completion of chemotherapy, an increase in the CTC and PSA levels was seen. These observations indicate that the CTC counts may provide independent prognostic information compared with the PSA level.

The average CTC size was smaller in men with more advanced disease. Chemotherapy altered the number but not the CTC size, as we did not find changes in the CTC size to suggest cellular degradation before, during, or after administration of chemotherapy.

We conclude that CTC counts can be reproducibly measured in patients with HRPC. The changes in CTC levels mirrored disease progression. The pattern and velocity of the CTC and PSA rise are different, suggesting that CTCs provide prognostic information independent of PSA. More importantly, the characterization of the CTC genotype and phenotype can guide future treatment and elucidate mechanisms of chemosensitivity and resistance.

### EXAMPLE 7

DISEASE ACTIVITY IS CORRELATABLE WITH NUMBER OF CIRCULATING EPITHELIAL CELLS IN COLON CANCER PATIENTS

The assay method of the present invention may be used to advantage in the assessment of patients with a variety of different cancer types. To illustrate, the method was also used to assess circulating epithelial levels in patients with colon cancer. There are over 130,000 new cases of colorectal cancer diagnosed yearly in the United States. 30-50% of these patients will recur and die of their disease. Rational development of new treatments is hindered by infrequent availability of tumor biopsy before and after treatment to document drug effect.

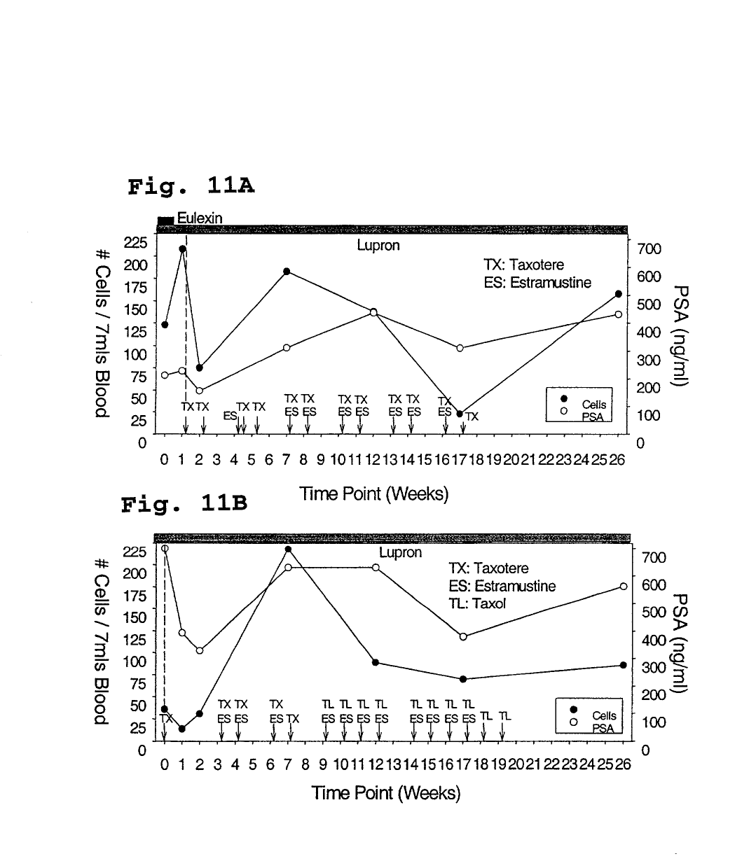

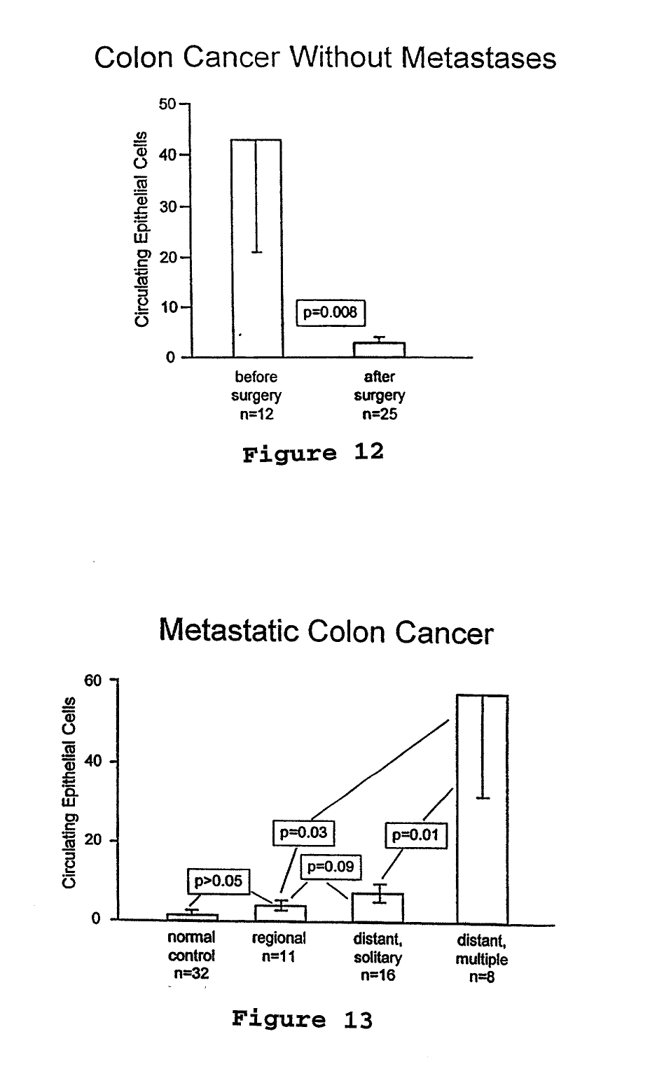

Colon cancer patients without evidence of metastases were evaluated for the presence of circulating epithelial cells before and after surgery. The results are shown in FIG. 12 and summarized in Table IX. The data reveal that the number of circulating epithelial cells in colon cancer patients is greater prior to surgical intervention.

TABLE VIII
CIRCULATING EPITHELIAL CELLS IN COLON CANCER PATIENTS
WITHOUT EVIDENCE OF METASTASES

| | | CIRCULATING EPITHELIAL CELLS | |
|---|---|---|---|
| | NUMBER OF | DETECTED BY FLOW CYTOMETRY IN | |
| TIME OF | PATIENTS | 10 ml OF BLOOD | |
| TESTING | TESTED | MEAN ± SEM | RANGE |
| Before surgery | 12 | 42.3 ± 22.0 | 0-234 |
| After surgery | 25 | 2.7 ± 0.7 | 0-15 |

Table X and FIG. 13 depict data obtained when colon cancer patients with evidence of metastases were assessed for the presence and number of circulating epithelial cells. The results revealed that the number of epithelial cells in peripheral blood is larger in patients with metastatic disease as compared to local disease after surgery. The results further show that the extent of metastatic disease may be correlated with the number of circulating epithelial cells.

TABLE IXA
CIRCULATING EPITHELIAL CELLS IN COLON CANCER PATIENTS
WITH EVIDENCE OF METASTASES

| | | CIRCULATING EPITHELIAL | |
|---|---|---|---|
| METASTATIC | | CELLS DETECTED BY FLOW | |
| STATUS OF | NUMBER OF | CYTOMETRY IN 10 ML OF | |
| PATIENTS | PATIENTS | BLOOD | |
| TESTED | TESTED | MEAN ± SD | RANGE |
| REGIONAL | 11 | 3.7 ± 0.6 | 1-6 |
| DISTANT, SOLITARY | 16 | 7.6 ± 2.0 | 0-21 |
| DISTANT, MULTIPLE | 8 | 54.0 ± 25.1 | 5-200 |
| NORMAL CONTROL | 32 | 1.0 ± 0.2 | 0-4 |

Rational clinical development of anticancer agents is impeded by infrequent access to repeat tumor biopsies for in vivo pharmacodynamic evaluation. The methods of the present invention overcome this limitation by permitting assessment of drug effect in circulating tumor cells. An additional pilot study to evaluate the ability of the immunomagnetic separation and automated fluorescent microscopic system of the invention to isolate, enumerate, and characterize circulating epithelial cells from the peripheral blood of patients (pts.) with metastatic colorectal cancer was performed. Twenty patients with measurable metastatic disease were enrolled. Fifty ml of peripheral blood were obtained at initiation of therapy and at disease reevaluation timepoints (6-10 week intervals). In addition, fresh tumor was obtained in four patients for comparison of circulating and in situ cancer cells by flow cytometry and gene array. Patient characteristics were: 7M/13F, median age 64 (range 41-80), median time with metastatic disease 2.7m (range 0.6-25m). Eleven patients had received prior chemotherapy for metastatic disease. Sites of metastatic disease included liver (13 patients), lung (8 patients), peritoneum (5 patients), small bowel, and anterior abdominal wall (1 patient each). Median diameter of largest metastatic lesion was 5 cm (range 1.5-12 cm). Circulating epithelial cells were purified from whole blood after labeling with anti-epithelial cell adhesion molecule (EpCAM) conjugated to ferrofluid. Median number of epithelial cells recovered was 7/7.5 ml peripheral blood (range 3 to 150) as determined by flow cytometric labeling with anti-cytokeratin. The results of this study are shown in Table IXB.

TABLE IXB
Characterization of circulating epithelial cells
from patients with metastatic colon cancer
Patient Characteristics (N = 20)

| Sex | 7 M/13 F |
|---|---|
| Fresh tumor available | 6 |
| Age (yrs.) | |
| Median | 64 |
| Range | 41-80 |
| Samples per patient (# pts.) | |
| 1 | 13 |
| 2 | 3 |
| 3 | 4 |
| Prior chemotherapy (# pts.) | |
| None | 7 |
| Adjuvant | 9 |
| Metastatic | 7 |
| adjuvant and metastatic | 4 |
| Time with metastatic disease (months) | |
| Median | 2.7 |
| Range | 0-25 |
| Sites of metastases (# sites) | |
| Liver | 13 |
| Lung | 8 |
| Peritoneum | 5 |
| Size of largest metastases (cm.) | |
| Median | 5 |
| Range | 1.5-12 |

Additional phenotyping by flow cytometry for epidermal growth factor receptor and thymidylate synthase expression to evaluate suitability of this technology for in vivo pharmacodynamic assessment can also be performed in accordance with the methods of the present invention. This study has demonstrated the feasibility of isolating circulating tumor cells from the blood of patients with metastatic colorectal cancer. Additionally, the present invention encompasses methods for assessing alterations in circulating tumor cells relative to tumor cells present in situ in a tumor mass. Such alterations may include for example, gain or loss of tumor diathesis associated molecules. Alterations in genotype or phenotype may also be examined.

The examples above demonstrate the highly significant differences in the number of circulating epithelial cells between healthy individuals and patients with breast, prostate and colon cancer. In addition, significant differences in the number of circulating epithelial cells were found between patients with no detectable spread, spread to local lymph nodes and distant metastasis (Racila et al., (1998), supra). Additionally, the number of epithelial cells in the blood of patients after surgical removal of a primary carcinoma of the breast was monitored over a one-year period. In some of these patients residual disease was detected. In patients with metastatic disease, the changes in peripheral blood tumor cell count correlated with the tumor load and response to treatment. The results of these studies reveal the potential of the cell-based assay of the present invention as an objective non-invasive tool to detect the presence of malignant disease and measure the activity of the disease. Cellular morphology and immunophenotype reveal the malignant nature of the isolated cells.

### EXAMPLE 8

TISSUE SOURCE IDENTIFICATION OF ISOLATED EPITHELIAL CELLS

All of the aforementioned studies in patients reveal that there is an excess of circulating epithelial cells in patients who have cancer, compared to normal individuals or patients without cancerous diseases, including benign tumors. It is essential, however, to prove that these excess circulating epithelial cells are, in fact, cancer cells. This was accomplished by performing an experiment in which immunomagneticallly purified epithelial cells from patients with or without cancer were cytospun onto a glass slide and treated with anti-mucin. In addition, normal epithelial cells that were obtained from foreskin and blood from normal individuals, both used as controls, were also cytospun. It is significant that the slides were coded and examined “blinded”, that the observer had training in pathology and that normal epithelial cells were included. As can be seen in FIG. 8, there is a marked difference between the isolated cancer cells versus normal epithelial cells. Normal epithelial cells have a low nuclear to cytoplasmic ratio, i.e., there is abundant cytoplasm and a relatively small nucleus. The nucleus shows a smooth distribution of chromatin. The cells do not stain with anti-mucin. In contrast, cells from two patients with breast cancer have very large nuclei and a small rim of cytoplasm. Additionally, the chromatin is disorganized as shown by the dark patches in the nucleus and the cells stain intensively with anti-mucin. The same is observed in cells from two patients with prostate cancer. A physician trained in pathology was shown coded slides from patients with and without cancer (total of 21 slides). The pathology-trained physician correctly identified bloods from all the controls as not having cancer cells and displayed no-intraobserved error when shown slides twice. In the cases of two patients with prostate cancer, tumor cells were not seen in the study. One slide was re-examined and tumor cells were observed. The cause of this discrepancy appears to be the amount of time spent scanning the cell smear. In summary, the cytomorphology and immunophenotype indicate that the excess epithelial cells present in the blood in patients with cancer are indeed cancer cells.

The experiments described above indicated that the methods disclosed herein enable the detection of cancer cells in the blood of patients with early tumors. Indeed, in 25 of 27 patients who were clinically determined to have organ-confined disease (early stage cancer), we detected the presence of cancer cells in the blood. This means that the assay should detect cancer cells much earlier in those solid tumors that are normally detected late (10⁹-10¹⁰ tumor cells). Moreover, the test should allow detection of breast, prostate and colon cancer earlier, perhaps before detection of a primary tumor by conventional means. The organ-origin of tumor cells in the blood for prostate can be established by staining with anti-prostate specific membrane antigen (PMSA), anti-PSA (prostate specific antigen), or other antibodies specific to the prostate in male subjects. For breast carcinoma in female patients, staining with anti-mammoglobin, anti-progesterone receptor, anti-estrogen receptor and anti-milk fat globulin antigen I and II will indicate a breast origin of tumor.

Our test should detect carcinoma cells from other organs, e.g., pancreas, esophagus, colon, stomach, lung, ovary, kidney, etc. The following table shows examples in which excess epithelial cells were observed in several patients with carcinomas other that the breast and prostate.

TABLE X

| NUMBER OF CELLS | CANCER |
|---|---|
| PER 20 ML BLOOD | DIAGNOSIS |
| 8 | Uterus adenocarcinoma |
| | (Stage 1B) |
| 11 | Head and Neck |
| | adenocarcinoma |
| 15 | Lung small |
| | undifferentiated |
| 14 | Neck Squamous cell |
| | carcinoma |

Each of the carcinomas described in the table above express tissue specific antigens whose corresponding antibodies can be used to determine the organ-origin of the circulating tumor cells.

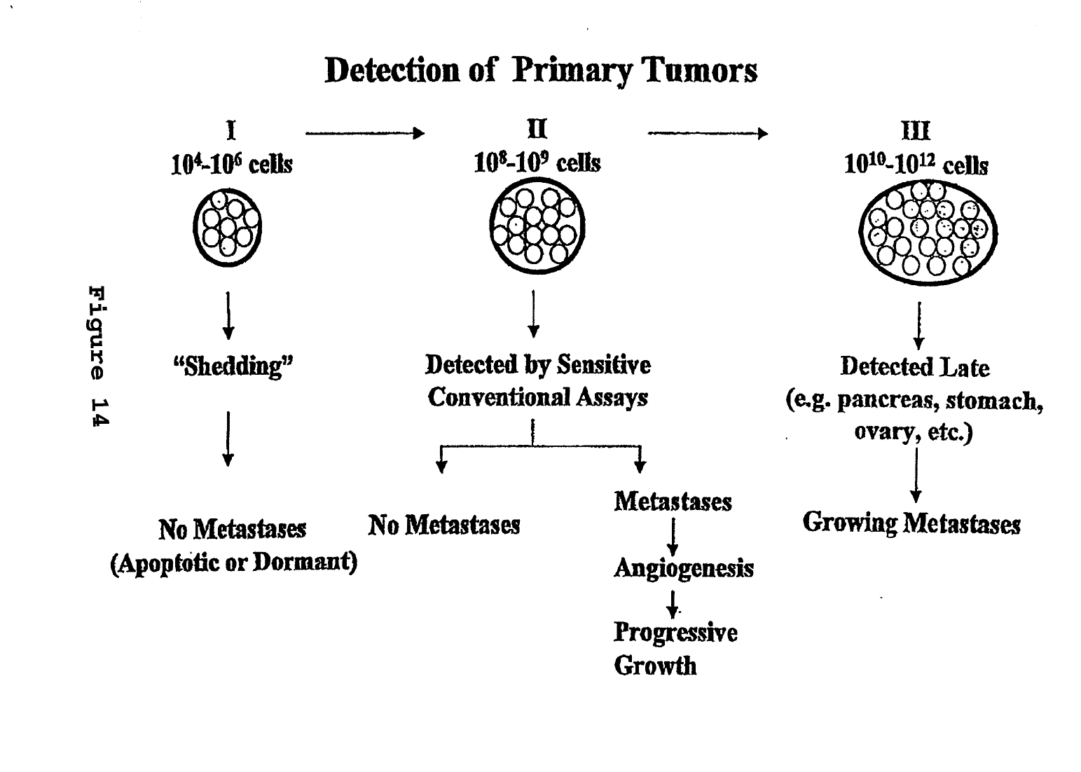

A diagram showing the process that provides primary tumor cells progress to metastatic cancer in FIG. 14. The blood test of the invention can also be used to detect cancer cells in patients previously treated successfully for cancer and now in long-term complete remission. Indeed circulating epithelial cells, i.e., dormant tumor cells, have been detected in patients treated five or more years previously and who appear to be clinically free of tumor. This explains why recurrence in patients can occur many years, even decades after apparently successful treatment. In fact, accumulating evidence suggests that the recurrence of breast cancer occurs at a slow steady rate approximately 10-12 years after mastectomy.

### EXAMPLE 9

DETECTION OF TUMOR CELLS IN THE BLOOD OF A PATIENT WITH HIGH PSA LEVELS AND A NEGATIVE BIOPSY

As indicated by the foregoing examples, the present invention may be used to advantage to diagnose cancer in presently asymptomatic patients. To illustrate this point, a patient with a two-year history of high PSA levels (>12 pg/ml), had a needle biopsy of the prostate performed two weeks prior to the analysis set forth below. The biopsy did not reveal the presence of malignancy. It is also noteworthy that a prior biopsy performed 18 months earlier was also negative.

Before obtaining a 20 ml blood sample, the patient was given a digital rectal exam and a gentle massage of his enlarged prostate with the intention of increasing the occurrence of tumor cells in the blood. The blood sample was enriched using the methods of the present invention. The enriched fraction was examined by microscopy employing a Wrights-Giemsa stain. Morphological examination of the isolated cells revealed their malignant character. Clearly this patient had cancer. Given the high PSA levels observed, a diagnosis of prostate cancer is likely. The origin of the cells may be determined using appropriate reagents as described herein. The results presented in this example reveal that the methods of the present invention can be used to detect cancers that might otherwise go undetected.

The notion of employing a localized massage to promote shedding of tumor cells into blood as a means of enhancing sensitivity of the blood test is a concept with considerable merit. Cells that are released into the circulation by this approach, following isolation may be used for a variety of different purposes. In the case of cells isolated with ferrofluids, isolated cells can be readily cultured and/or cloned. The resultant cell lines can be used to assess a variety of malignant cell characteristics such as chemotherapeutic sensitivity and growth factor dependency.

### EXAMPLE 10

MONITORING BIOCHEMICAL ALTERATIONS IN ISOLATED CIRCULATING TUMOR CELLS

During the development of cancer, genetic instability of the tumor cells results in the generation of new clones with selective growth advantages that can lead to a change in the predominant phenotype of the tumor cells over time. One such example is the amplification of the HER-2 (c-erbB2) proto-oncogene, which results in the over-expression of the encoded epithelial growth factor transmembrane protein receptor. The objectives of this study were to determine whether HER-2 receptor could be quantified on circulating tumor cells (CTCs) in the blood of patients with advanced breast cancer and whether the pattern of HER-2 expression changed during the course of treatment. This information should enable the clinician to predict the outcome of costly new target-directed therapies. While HER-2 is exemplified herein, it is highly desirable that additional tumor-diathesis associated molecules on or in the tumor cells be identified and assessed in this manner. Suitable tumor diathesis associated molecules that may be assessed following isolation of circulating tumor cells are set forth in Table XI. Exemplary approaches for detecting alterations in tumor diathesis associated molecules such as nucleic acids or polypeptides/proteins associated with malignancy include:

a) comparing the sequence of predetermined nucleic acid in the sample with the corresponding wild-type nucleic acid sequence to determine whether the sample from the patient contains mutations; or

b) determining the presence, in a sample from a patient, of tumor diathesis associated molecules polypeptide and, if present, determining whether the polypeptide is full length, and/or is mutated, and/or is expressed at the normal level; or

c) using DNA restriction mapping to compare the restriction pattern produced when a restriction enzyme cuts a sample of tumor diathesis associated molecules nucleic acid from the patient with the restriction pattern obtained from the cognate normal gene or from known mutations thereof; or,

d) using a specific binding member capable of binding to a tumor diathesis associated molecules nucleic acid sequence (either normal sequence or known mutated sequence), the specific binding member comprising nucleic acid hybridizable with the sequence, or substances comprising an antibody domain with specificity for a native or mutated nucleic acid sequence or the polypeptide encoded by it, the specific binding member being labeled so that binding of the specific binding member to its binding partner is detectable; or,

e) using PCR involving one or more primers based on normal or mutated gene sequence to screen for normal or mutant gene sequences in a sample from a patient.

Alterations in protein molecules, e.g., those arising from deletion or point mutation in the encoding nucleic acids may be assessed using conventional methods which are well known to those of ordinary skill in the art. Such methods include gel electrophoresis, western blotting, HPLC, and FPLC. Alterations in nucleic acid molecules which are associated with malignancy may also be assessed using conventional methods.

Alterations in carbohydrate moieties present on membrane glycoproteins may also be assessed. Protocols for analyzing glycoconjugates and the sugars thereon are provided in Chapter 17 of Ausubel et al., supra.

In the methods of the invention, surgically resected primary tumor tissue is assessed for the expression of a limited number of genes and/or proteins. Using breast cancer as an example, such tumor diathesis associated molecules may include receptors for HER-2, estrogen, and progesterone. Problems arise as the disease progresses, since changes in the phenotype of the tumor cells often occur after the original diagnosis, and resistance to a treatment can only be inferred after the treatment has failed. Assessing the presence of target tumor diathesis associated molecules on CTCs before and during treatment constitutes a real-time, “whole body” biopsy of the tumor. With these goals in mind, an assay capable of both enumerating CTCs as well as quantifying and characterizing the expression of tumor diathesis associated molecules present on or in tumor cells was developed. In the present example, we describe changes in the expression of HER-2 on CTCs from the blood of patients with metastatic carcinoma of the breast.

The following materials and methods are provided to facilitate the practice of Example 10.

Patient Population

The study was conducted at the Lombardi Cancer Center of Georgetown University. Patients in this study were selected from a larger group of 24 women with stage III or metastatic breast cancer who were enrolled in a pilot study monitoring CTC fluctuations during therapy (Walker et al. Proc. Am Soc. Clin. Onc. (2001) 19-54b). Nineteen patients, whose primary tissue blocks could be obtained, were included in this study. All patients had measurable disease and were either newly diagnosed stage III patients about to start neo-adjuvant chemotherapy or patients with documented progressive metastatic breast cancer who were to begin a new endocrine, chemo, or experimental therapy. Choice of therapy was left to the discretion of the treating physician. Neither the treating physician, nor the patient, was informed of the CTC assay results. As a control, blood specimens were collected from twenty-two disease-free women, 21 years or older. The protocol was IRB-approved. Informed consents or releases were obtained from patients and normal control subjects respectively.

Clinical Assessments

When possible, two pre-enrollment evaluations were performed, the first within 4 weeks of study entry and the second (baseline) immediately prior to commencing treatment. A current medical history was taken and physical exams were performed by a clinical oncologist. Hematology analysis included CBCs with differential and platelet count; biochemistry, including urine analysis, BUN, GOT, GPT, LDH, creatinine, alkaline phosphatase, and total/direct bilirubin. Tumor assessments were based on physical measurements (caliper or ruler) and/or imaging studies (CT scan, MRI, Bone scans, etc). Evaluation of response to therapy was defined using Union International Contra Cancer (UICC) criteria (Monfardini et al. eds. UICC-Manual of Adult and Pediatric Medical Oncology, Berlin, Germany, Springer 1987, p22-38) and was carried out without knowledge of CTC/CTC HER-2 results. Timing of the blood draws and evaluation of clinical status depended on the individual treatment protocol. All blood samples were drawn in 10 ml ACD Vacutainer tubes (Becton-Dickinson, N.J.), maintained at room temperature, and shipped overnight to Immunicon. Samples were processed within 24 hours after collection.

HER-2 staining tissue blocks

Tissue blocks from the patient's primary tumors were collected and slides were prepared from paraffin-embedded tissue sections. The slides were evaluated for HER-2 expression using the HercepTest® (DAKO, Carpinteria, Calif. according to the manufacturer's instructions. Positive and negative slides were reviewed by a single pathologist and scored according to the manufacturer's guidelines using a scale from 0 to 3+.

Sample preparation

Five mls of blood were transferred to disposable tubes with an internal diameter of 17 mm (Fisher Scientific, USA) and centrifuged at 800 g for 10 minutes with the brake off. Phosphate Buffered Saline (PBS) with Bovine Serum Albumin was added to bring the volume up to 10 ml and the sample was mixed by inversion. As mentioned previously, monoclonal antibodies (Mabs) specific for epithelial cell adhesion molecule (EpCAM) are broadly reactive with tissue of epithelial cell origin. The Mab VU-1D9 recognizes EpCAM and was coupled to magnetic nanoparticles (ferrofluids, Immunicon, Huntingdon Valley, Pa.). To increase the 'magnetic loading' of the EpCAM⁺ cells and decrease the variability in capture efficiency due to differences in the EpCAM density on the cell surface, desthiobiotin was coupled to EpCAM-labeled magnetic nanoparticles to form CA-EpCAM as described in the previous examples. CA-EpCAM ferrofluid and a buffer containing streptavidin were then added to the sample to achieve this increase in the magnetic labeling of the cells. Desthiobiotin on the CA-EpCAM ferrofluid was subsequently displaced by biotin, which is contained in the permeabilization buffer described below, thereby reversing the cross linking between the CA-EpCAM ferrofluids. The sample was immediately placed in a magnetic separator composed of four opposing magnets for 10 minutes (QMS17, Immunicon, Huntingdon Valley, Pa.). After 10 minutes, the tube was removed from the separator, inverted 5 times, and returned to the magnetic separator for an additional 10 minutes. This step was repeated once more and the tubes were returned to the separator for 20 minutes. After separation, the supernatant was aspirated and discarded. The tube was removed from the magnetic separator and the fraction collected on the walls of the vessel was resuspended with 3ml of BSA containing PBS. The suspension was placed in the magnetic separator for 10 minutes and the supernatant was aspirated and discarded. The cells were resuspended in 200 μl of a biotin containing permeabilization buffer (Immuniperm, Immunicon Corp.) to which Mab-fluorochrome conjugates were added at saturating conditions. The Mabs consisted of a Phycoerythrin (PE) conjugated anti-cytokeratin Mab Cll recognizing keratins 4,6,8,10,13, and 18, (Immunicon Corp.), Peridinin Chlorophyll Protein (PerCP)-labeled anti-CD45 (Hle-1, BDIS, San Jose, Calif.) and cyanin 5 (CY5)-labeled anti-HER-2. The MAb anti-HER-2, designated HER-81, recognizes an epitope on the extracellular domain of HER-2 and does not cross block with trastuzumab or its murine parent 4D5. It is a murine IgG_{1K} with a Kd of 10⁻¹⁰M on BT474 breast carcinoma cells. After incubating the cells with the Mabs for 15 minutes, 2 ml of cell buffer (PBS, 1%BSA, 50 mM EDTA) was added and the cell suspension was magnetically separated for 10 minutes. After discarding the non-separated suspension, the collected cells were resuspended in 0.5 ml of PBS to which the nucleic acid dye used in the Procount system was added (Procount, BDIS, San Jose Calif.). In addition 10,000 fluorescent counting beads were added to the suspension to verify the analyzed sample volume (Flow-Set Fluorospheres, Coulter, Miami, Fla.).

Cell Lines

Cells of the prostate cancer cell line PC-3 and the breast cancer line SKBR-3 were cultured in flasks containing 10 ml RPMI-1640 supplemented with 10%FCS. Cells were harvested from the flasks after trypsin treatment, washed and resuspended to obtain the desired cell concentration. For quantitative assessment of HER-2 density on both cell lines, 20,000 cells were stained with the Mab HER-81 conjugated to PE (HER-2 PE). For calibration of expression levels of HER-2 100 μl of cell suspension containing approximately 3,000 cells was spiked into 5 ml of blood.

Sample analysis

Samples were analyzed on a FACSCalibur flow cytometer equipped with a 488 nm Argon ion laser and a 635 nm laser diode (BDIS, San Jose, Calif.). Data acquisition was performed with CellQuest (BDIS, San Jose, Calif.) using a threshold on the fluorescence of the nucleic acid dye. The acquisition was halted after 8000 beads or 80% of the sample was analyzed. Multiparameter data analysis was performed on the listmode data (Paint-A-Gate^{Pro}, BDIS, San Jose, Calif.). Analysis criteria included size defined by forward light scatter, granularity defined by orthogonal light scatter, positive staining with the PE-labeled anti-cytokeratin MAb and no staining with the PerCP-labeled anti-CD45 Mab. For each sample, the number of events present in the region typical for epithelial cells was multiplied by the correction factor 1.25 to account for the sample volume not analyzed by the flow cytometer. The flow cytometer was calibrated to assess the density of HER-2 on cells using phycoerythrin (PE)-labeled beads with known numbers of fluorochrome molecules (QuantiBRITE PE, BDIS, San Jose, CA). The densities of HER-2 on the breast cancer cell line, SKBR-3, and prostate cancer cell line, PC-3 were then assessed by measuring the fluorescence intensity of cells treated with PE-anti-HER-2 under saturating conditions. The average numbers of HER-2 receptors on SKBR-3 and PC-3 cells were 850,000 and 9,500, respectively.

Enumeration of CTCS.

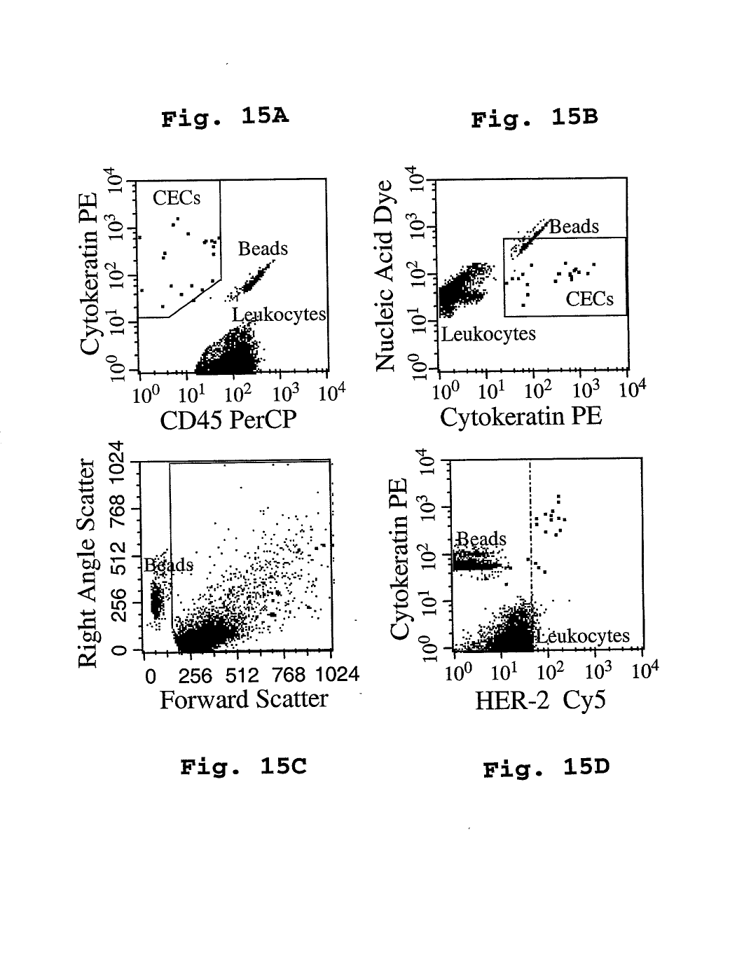

To obtain the sensitivity needed for the enumeration of CTCs, a combination of technologies was required. First, an enriched sample of EpCAM+cells in the blood was prepared by mixing the cells with colloidal paramagnetic particles coated with MAbs specific for EPCAM, followed by magnetic separation. This immunomagnetic sample enrichment is performed to reduce both the sample volume and the number of background hematopoietic cells. The remaining cells were then labeled with PE-anti-cytokeratin to identify epithelial cells, PerCP-anti-CD45 to identify leukocytes, Cy5-anti HER-2 to determine the expression of HER-2, and a nucleic acid dye was added to exclude residual erythrocytes, platelets and other non-nucleated “events”. Magnetic separation (i.e., washes) was used throughout the sample preparation to eliminate excess labeling reagents, to reduce carryover of non-target cells, and to permit the resuspension of cells in the desired volume. The samples were analyzed by flowcytometry with each event being characterized by two light scatter and four fluorescence parameters. FIG. 15 (Panels A-D) is an example of the flowcytometric analysis of a 5 ml blood sample obtained from a patient with metastatic breast carcinoma. Panel 15A shows the correlative display of PerCP-anti-CD45 versus PE-anti-Cytokeratin; Panel 15B shows the PE-anti-cytokeratin versus Nucleic Acid Dye; Panel 15C shows the forward and right angle scatter, and Panel 15D shows the Cy5- anti-HER-2 versus PE-anti-Cytokeratin. The location of beads and leukocytes are indicated in the Panels and are depicted in black. The gates drawn in Panels 15A, 15B and 15C indicate the regions typical for CTCs (CD45⁻, Cytokeratin+, >4 μm, Nucleic acid⁺). To qualify as CTCs (highlighted black small squares) events had to fall within all three regions. All other events consisted of debris and are depicted in gray. CTCs were considered to be HER-2⁺ if they exceeded the background staining of leukocytes as indicated by the dotted line in Panel 15D. The same gates shown in the figure were used to analyze all the samples in this study. In 5 ml of blood from 22 controls 1.5±2.1 events per subject were detected in the regions that classified as CTCs. None of these events were associated with expression of HER-2. In contrast 5-214 CTCs/5 ml were detected in 10 of 19 blood samples from patients with measurable disease who were starting an initial or new line of therapy.

Expression of HER-2 on CTCs

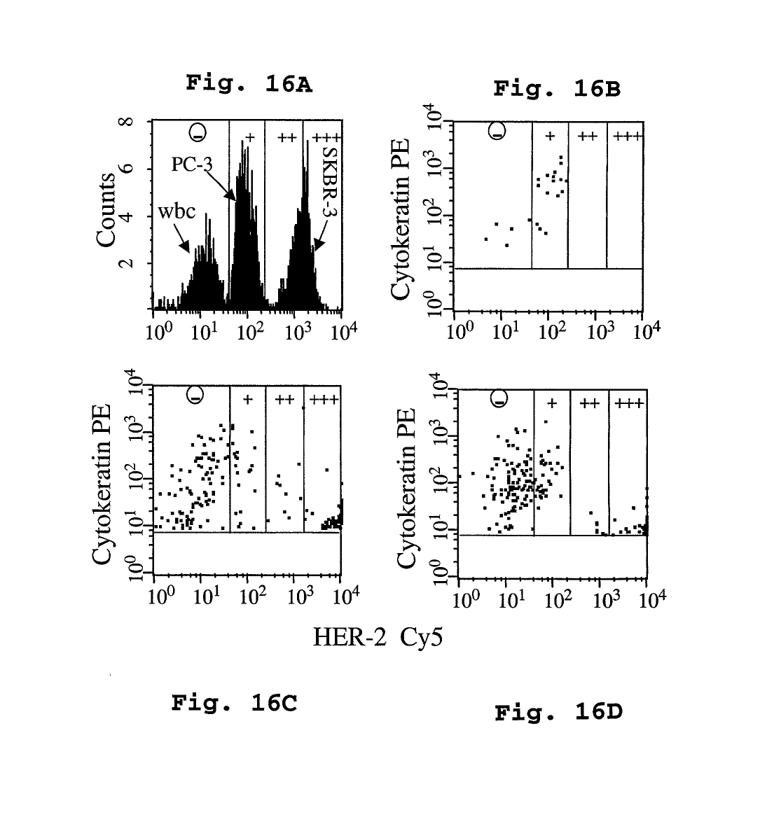

To calibrate expression levels of HER-2 on CTCs, 5 ml of blood was spiked with SKBR-3 (˜850,000 HER-2 receptors) and PC-3 (˜9,500 HER-2 receptors) cells and processed for CTC analysis. Leukocytes, PC-3 and SKBR-3 cells were identified based on unique profiles of cytokeratin and CD45 expression. The fluorescence intensity of the Cy5-anti- HER-2 staining of each of the cell populations is shown in FIG. 16A. The levels of expression of HER-2 on CTCs were subdivided into four categories; (1) no expression, [below 5,000 receptors (−)] typical of leukocytes, (2) low expression [between 5 5,000 and 50,000 receptors (+)] typical of PC-3, (3) medium expression [between 50,000 and 500,000 receptors (++)], and (4) high expression [>500,000 receptors (+++)] typical of SKBR-3. The expression of cytokeratin and HER-2 on CTCs from three breast cancer patients is shown in FIG. 16B, 16C and 16D. Most of the CTCs from the patient shown in panel 16B had low levels of expressed HER-2 but a few lacked HER-2 altogether. All levels of HER-2 expression were found on the CTCs of the patient illustrated in Panel 16C. The CTCs in the patient shown in Panel 16D appeared to separate into CTCs expressing no or low levels of HER-2 and CTC expressing high levels of HER-2. In all cases, CTCs that expressed high levels of HER-2 expressed relatively low levels of cytokeratin. Table XI shows the treatment modality, response to therapy, HER-2 expression on primary tissue, months between the tissue biopsy and the assessment of CTC, baseline and post-treatment CTCs and expression of HER-2 on CTCs from 19 breast cancer patients. Tissue blocks of the primary tumor obtained at the time of diagnosis were assayed for HER-2 expression. The time between diagnosis 5 (tumor biopsy) and CTC determination varied greatly (Table XI). The tissue blocks of seven of the 19 patients were positive for HER-2 (2+or 3+) by Herceptest staining. In 6/7 patients, CTCs were detected and in all 6, the CTCs expressed HER-2, (Table XII). In one patient, CTCs expressed HER-2 whereas the tissue section did not. The percentage of CTCs that expressed HER-2 and the density of the HER-2 on the surface of the CTCs varied considerably.

TABLE XI
Summary of data on patients with advanced breast cancer.

| | | | | Time | | | | |
|---|---|---|---|---|---|---|---|---|
| | | | | (month) | | Post | Baseline | Post Rx |
| Pt | | | Tissue | Tissue/CT | Baseline | Rx | HER-2 on | HER-2 on |
| # | Response | Rx | HER-2 | Cs | CTCs | CTCs | CTCs | CTCs |
| 23 | P | Exp | 2+ | 32 | ++ | +++ | +++ | ++ |
| 25 | P | Exe | 2+ | 74 | ++ | +++ | + | ++ |
| 16 | P | Cap | 2+ | 63 | ++ | +++ | − | ++ |
| 2 | P | Do | 0 | 40 | + | − | ++ | na |
| 7 | P | Exp | 0 | 25 | + | +++ | − | +++ |
| 22 | P | Meg | 0 | 142 | + | +++ | − | ++ |
| 6 | P | Tam | 0 | 40 | − | − | na | na |
| 8 | P | Exp | 0 | 7 | − | − | na | na |
| 19 | S | Go/A | 3+ | 12 | ++ | − | +++ | na |
| | | n | | | | | | |
| 21 | S | Tr/T | 2+ | 156 | +++ | ++ | +++ | ++ |
| | | a | | | | | | |
| 20 | S | Flu | 2+ | 19 | ++ | +++ | + | + |
| 5 | S | Exp | 0 | 78 | − | − | na | na |
| 13 | R | Do/C | 3+ | <1 | − | − | na | na |
| | | y | | | | | | |
| 28 | R | Do/C | 0 | <1 | + | − | − | na |
| | | y | | | | | | |
| 27 | R | Exp | 0 | 73 | − | − | na | na |
| 3 | R | Do/C | 0 | 1 | − | − | na | na |
| | | y | | | | | | |
| 9 | R | Do/C | 0 | <1 | − | − | na | na |
| | | y | | | | | | |
| 26 | R | Exe | 0 | 36 | − | − | na | na |
| 14 | R | Do/C | 0 | 72 | − | − | na | na |
| | | y | | | | | | |
| Pt# = patient number. Response Category: P = progressed, R = complete or partial response, S = stable. Rx = Treatments: Do = doxorubicin, Cy = cyclophosphamide, Flu = fluoxymesterone, Go = goserelin acetate, An = anastrozole, Exe = exemestane, Meg = megestrol acetate, Tam = tamoxifen, Cap = capecitabine, Ta = Paclitaxel, Tr = trastuzumab; Time (month) tissue/CTCs = month between tissue | | | | | | | | |
| #biopsy and CTC assessment. | | | | | | | | |
| CTC: − = <5 CTC/5 ml blood; + = 5-10 CTC/5 ml blood; ++ = 10-100 CTC/5 ml blood; +++ = 100-1000 CTC/5 ml blood. | | | | | | | | |
| HER-2 on CTC: − = <25% of CTC express HER-2; + = 25-50% of CTC express HER-2; ++ = 50-75% of CTC express HER-2; +++ 75-100% of CTC express HER-2; na = not applicable as no CTC were detected. For the purpose of an overview, we have classified HER-2 expression as the percentage of CTCs expressing HER-2. In the subsequent figures the actual number of CTCs at the four different densities is shown at the different | | | | | | | | |
| #time points. | | | | | | | | |

CTC counts and expression of HER-2 on CTCs during treatment.

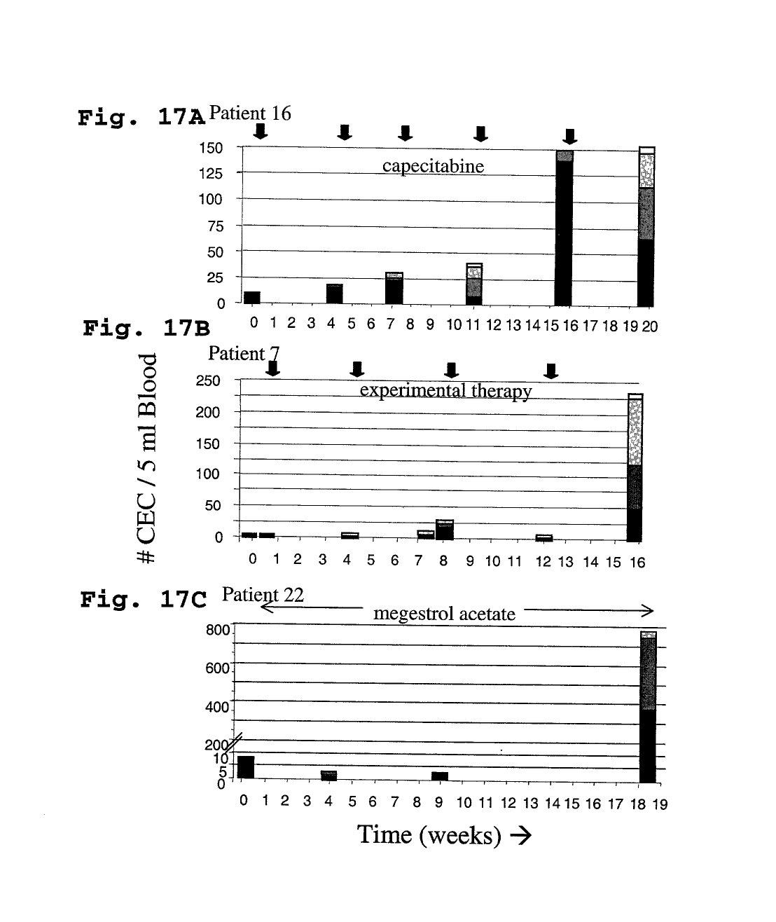

Eight patients progressed, 4 had stable disease, and 7 responded to their therapy (Table XI). In three patients, no HER-2 expression was detected on the CTCs at the initiation of treatment. The disease progressed in all three patients during the time they were being monitored. During subsequent measurements, CTCs increased and a proportion of the CTCs expressed HER-2 (Table XI). FIG. 17 shows the number of CTCs detected in these three patients before and during treatment. The arrows indicate the time of treatment. The number of CTCs that lacked (0) HER-2 or that expressed low (+), medium (++) or high (+++) levels of HER-2 is indicated within the bars.

In three patients with disease progression, HER-2 [symbol] HER-2⁻, [symbol] HER-2⁺, [symbol] HER-2⁺⁺ [symbol] HER-2⁺⁺⁺).

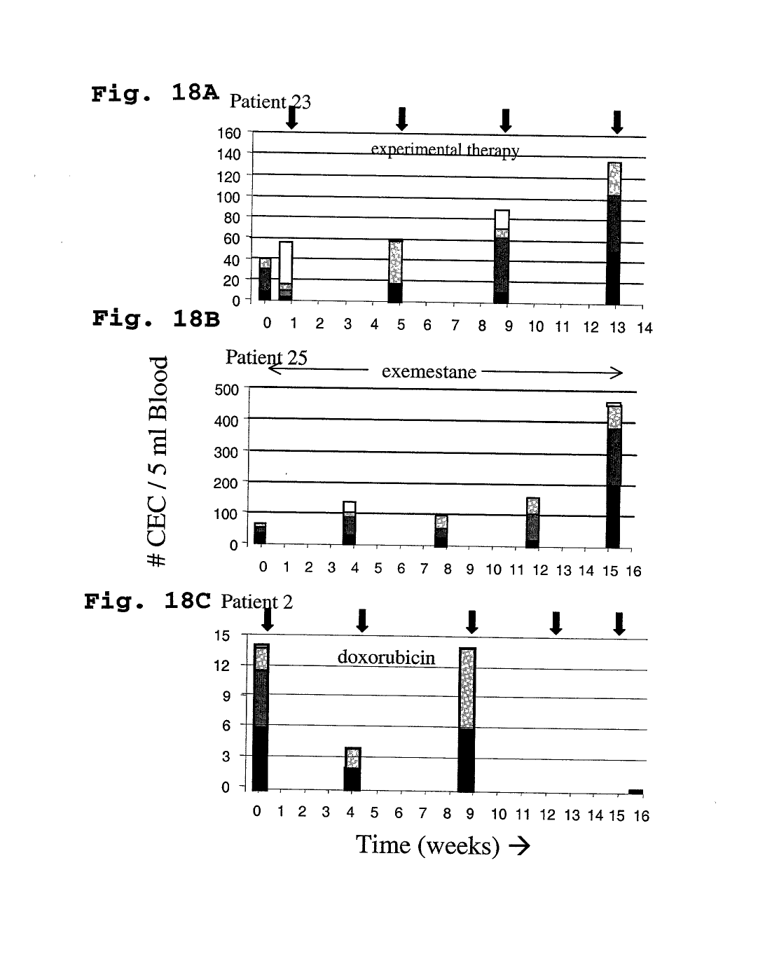

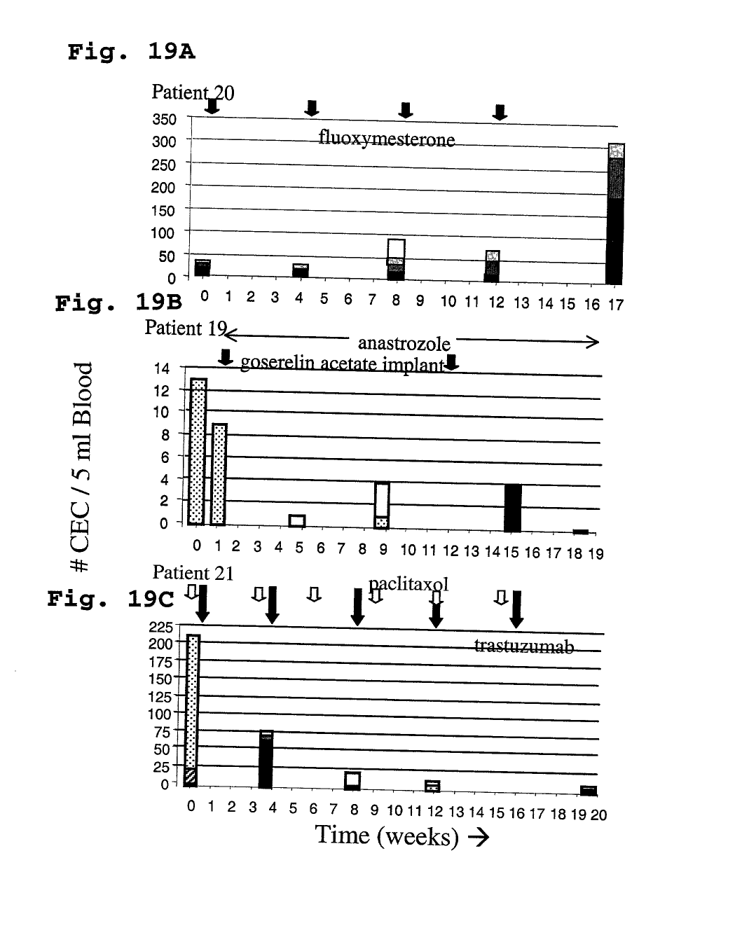

was present on CTCs at baseline and the percentage of CTCs that expressed HER-2 did not show pronounced changes (FIG. 18). The CTCs increased steadily during the course of treatment in the patients shown in FIG. 18A and 18B. The CTCs in the blood of the patient shown in Panel 18C were substantially lower and did not increase during the course of treatment. The number of CTCs detected before, during and after treatment of another three patients who had stable disease during follow up is shown in FIG. 19. The number of CTC in the blood of the patient shown in FIG. 19A increased whereas the CTCs in the other two patients decreased but were still detectable after treatment. Of interest, the patient illustrated in FIG. 19C (patient 21), was treated with trastuzumab and paclitaxel. 98% of the CTCs expressed low to medium levels of HER-2 before treatment. The number of CTCs decreased from 214 to 79 four weeks after initiation of treatment and only 14% expressed HER-2. The number of CTCs continued to decrease during the treatment course (23, 13 and 11 CTCs) and the percentage of CTCs that expressed HER-2 increased to 78, 100 and 54% respectively. The patient's clinical status (stable disease), was based on CT and bone scans taken after the course of treatment although a clear reduction in CTCs was observed.

Discussion

The recent approval of trastuzumab (Herceptin®) for the treatment of women with HER-2-overexpressing breast cancer has added an important regimen to the therapies available for patients with this disease (Baselga et al. Proc. Am. Soc. Clin. Onc. (1995) 14:103a; Pegram et al. J. Clin. Oncol. (1998) 16:2659-71; Cobleigh et al. J. Clin. Oncol. (1999) 17:2639). The development and utilization of such target-directed therapies requires a ‘real time’ accurate, sensitive, specific and reliable in-vitro diagnostic assay. The assay must be capable of detecting not only the patient subpopulation likely to benefit from a given therapy but the patient subset in whom resistance to that treatment has developed. Candidacy for trastuzumab therapy currently requires a positive diagnosis by either by immunohistochemistry (positive HER-2 staining of the tumor) or evidence of amplification of the HER-2 gene as determined by fluorescence in-situ hybridization (FISH). Apart from the controversy concerning the accuracy and sensitivity of both techniques,(Lebeau et al., J. Clin. Oncol. (2001) 19:354-363; Kakar et al. Molecular Diagnosis (2000) 5:199-207) neither technique detects a change in the tumor phenotype from the time of initial diagnosis to the time of detection of recurrence of the disease. Changes in tumor genotype and phenotype occurring during the clinical course of the disease have been documented (Vogelstein et al. N. Engl. J. Med. (1988) 319: 525-532; Pihan et al. Cancer Res. (2001) 61:2212-2219) and should be determined before commencing costly target-directed therapy. Since most (>75%) breast cancer metastasis are internal (bone, liver, lung, etc) the morbidity and cost of obtaining routine biopsies is prohibitive. In the present example, we have shown that the presence of a tumor diathesis associated molecule can be quantitatively analyzed using epithelial cells isolated from blood. The increased number of these cells in patients with carcinoma indicates that they represent tumor cells. As demonstrated herein, the number of CTCs in blood can be used to assess tumor progression. Previous studies by the present inventors have revealed that CTCs measured at several time points during the day did not change, whereas substantial increases were found over a longer period of time during which the disease progressed. In the present study, CTCs were detected in 5 ml of blood from 10 of 19 patients with stage III and IV breast cancer. To increase the frequency of patients in whom CTCs are detected, larger blood volumes and automation of the sample preparation procedure can be used to increase the sensitivity of the assay. The observation that CTCs from patients with HER-2-overexpressing tumors were HER-2⁺ supports the rationale of the assay. More important was the fact that in three patients, a phenotypic conversion from HER-2⁻ to HER-2⁺ CTCs was observed. HER-2 overexpression was not detected on the CTCs prior to initiation of a new line of treatment but was detected on CTCs that underwent a concomitant substantial increase in number during the course of treatment. The conversion to HER-2⁺ CTCs might signify conversion to a more aggressive phenotype. The observation that expression of HER-2 was inversely related to expression of cytokeratin, a cytoskeletal protein associated with cellular differentiation supports this hypothesis (Schaafsma et al. in Rosen, P. P., Fechner, R. E., eds. Pathology Annual vol. 29 (1994) pp. 21-62) Thus, the ability to detect changes in HER-2 expression is of clinical importance in regards to selection of HER-2 targeted therapy and chemotherapy.

In summary, we have shown that the clinical status of patients with breast cancer can be evaluated by changes in the levels of CTCs and that antigen targets on these cells can be quantitatively assessed. This information might be of benefit in attempting to “Tailor” treatment for the individual patient's disease. While HER-2 is exemplified herein, alterations in many other tumor diathesis associated molecules and markers can occur as a tumor cell becomes more malignant. Molecules that exhibit alterations associated with malignancy include without limitation, mdr, thymidylate synthase, FSFR, p53, ras oncogenes, CD 146, src, MUC1, uPA, PAI-1, ACT and many others. Chromosomal translocations, point mutations in key cellular signaling molecules, and surface carbohydrate changes associated with cancer have all been previously described. Table XII provides a list of biological molecules that may be assessed by the clinician, provide a more accurate diagnosis of the patient's condition and, more importantly, to devise the appropriate therapeutic regimen for treatment. For example, pS2/pNR-2+ breast cancer indicates that the patient is likely to respond to endocrine therapy. Additional tumor diathesis associated molecules which are often altered during malignant progression and which may be analyzes in isolated circulating tumor cells are listed below. This list is meant to be illustrative only, and not limited to the molecules set forth.

TABLE XII
Theraputic Targets

Hormone & Hormone Regulated Proteins
Androgen Receptor
Cathepsin D
Estrogen Receptor
Estradiol
Progesterone Receptor
Somastatin
SRC1 = Steroid Receptor Coactivator-1
Onco/Suppressor proteins
Her-2 (cERB-b)
EGFR
ras
c-fos
c-jun
c-myc
p53
p63
nm23/NDP Kinase
PTEN/MMAC1
SMAD4/DPC4
Notch-1
JAK3
Cell Cycle & Proliferation
Cyclin A
Cyclin B
Cyclin C
Cyclin D
Cyclin E
Ki67
MDR/MRP proteins
Other targets
PSA
Prostatic Acid Phosphotase
CA 125
CA 15-3
CA 27-29
HGC
Cystic Fibrosis Transmembrane Regulator
Laminin Receptor
Neuron Specific Enolase (NSE)
Alpha Fetoprotein
CD99/MIC2
DHEA
Prolactin
CD66e/CEA
Filaggrin (epidermal cells)
Renal Cell Carcinoma (gp200)
TAG72/CA72-4
UPA-receptor (CD87)
Heregulin
IPO-38
Thymidylate Synthase
Topoisomerase Iia
Glutathion S Transferase (GST)
Lung-Resistance related Protein/Major Fault Protein
(LRP/MFP)
06-Methylguanine-DNA methyltransferase (MGMT)

Methods are available to the skilled artisan to analyze alterations in expression levels, metabolic function, and/or genetic alterations in the tumor diathesis associated molecules listed in XII. Changes in protein expression levels may be assessed via flowcytometry, laser scanning cytometry, immunocytochemistry, CellTracks, etc. Genetic changes such as point mutations can be assessed using restriction enzyme digestion, FISH, PCR, or Southern hybridization. Changes in glycosylation of glycoproteins and glycolipids is a common feature of cancer and may influence cancer cell behavior, perhaps by enabling cell-cell interactions which favor metastasis or by allowing cancer cells to evade immuno-surveillance. Alterations in glycosylation human cancer may be assessed using immunohistochemical techniques, mass spectometry, and column chromatography.

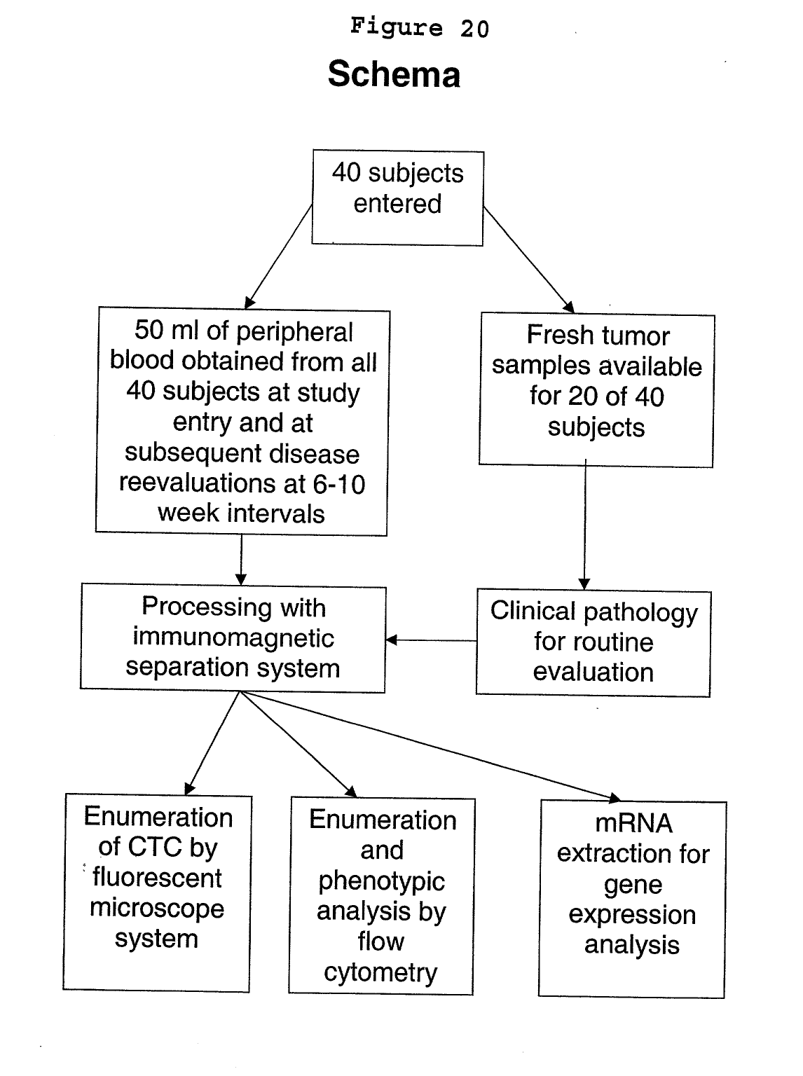

A schematic protocol for practicing the methods of the present invention is provided in FIG. 20.

### EXAMPLE 11

Tests Kits for diagnosing various aspects of cancer.

Also contemplated for use in the present invention are test kits comprising the reagents used to perform the assay of the invention. Such kits are designed for particular applications. Reagents may be assembled to facilitate screening of patients for circulating rare cells, including but not limited to tumor cells. In this embodiment, the kits contain colloidal magnetic particles comprising a magnetic core material, a protein base coating material and a biospecific ligand which binds specifically to a characteristic determinant present on the cancer cell to be isolated. The kit also includes at least one additional biospecific reagent that has affinity for a second characteristic determinant on the cancer cell to be isolated which differs from the determinant recognized by the biospecific ligand. The kit also includes a cell specific dye for excluding non-nucleated cells and other non-target sample components from analysis. An exemplary kit also comprises reagents for detecting at least one tumor diathesis associated molecule. Also provided in the kit is a Cell Spotter or Cell Tracks cartridge as described in Example 2.

A typical kit according to this invention may include anti-EpCAM coupled directly or indirectly to magnetic nanoparticles, and a pair of monoclonal antibodies, the first antibody recognizing a cancer specific determinant and the second antibody having affinity for a non-tumor cell determinant, e.g., a pan leukocyte antigen. Reagents which also detect at least one tumor diathesis associated molecule are also provided in the kit. The kit also contains a nucleic acid dye to exclude non-nucleated cells from analysis. The kit of the invention may optionally contain a biological buffer, a permeabilization buffer, a protocol, separation vessels, analysis chamber, positive cells or appropriate beads and an information sheet.

The kits described above may also be produced to facilitate diagnosis and characterization of particular cancer cells detected in circulation. In this embodiment, the kits contain all of the items recited above, yet also preferably contain a panel of cancer specific monoclonal antibodies. Using breast cancer as an example, a kit for diagnosis may contain anti-MUC-l, anti-estrogen, anti-progesterone receptor antibodies, anti-CA27.29, anti-CA15.3, anti-cathepsin D, anti-p53, anti-urokinase type plasminogen activator, anti-epidermal growth factor, anti-epidermal growth factor receptor, anti-BRCA1, anti-BRCA2, anti-prostate specific antigen, anti-plasminogen activator inhibitor, anti-Her2-neu antibodies or a subset of the above.

A kit is also provided for monitoring a patient for recurring disease and/or residual cells following eradication of the tumor. In this embodiment, the type of cancer will already have been diagnosed. Accordingly, the kit will contain all of the reagents utilized for screening biological samples for cancer yet also contain an additional antibody specific for the type of cancer previously diagnosed in the patient. Again using breast cancer as an example such a kit might contain anti-MUC-1. Alternatively, the kit may contain anti-Her2-neu.

The kits of the invention may be customized for screening, diagnosing or monitoring a variety of different cancer types. For example, if the kits were to be utilized to detect prostate cancer, the antibodies or complementary nucleic acids included in the kit would be specific for target molecules present in prostate tissue. Suitable antibodies or markers for this purpose include anti-prostate specific antigen, free PSA, prostatic acid phosphatase, creatine kinase, thymosin b-15, p53, HPC1 basic prostate gene and prostate specific membrane antigen. If a patient were to be screened for the presence of colon cancer, an antibody specific for carcinoembryonic antigen (CEA) may be included in the kit. Kits utilized for screening patients with bladder cancer may contain antibodies to nuclear matrix protein (NMP22), Bard Bladder tumor antigen (BTA) or fibrin degradation products (FDP). Markers and tumor diathesis associated molecules are known for many different cancer types.

The cells isolated using the kits of the invention may be further studied for morphology, RNA associated with the organ of origin, surface and intracellular proteins, especially those associated with malignancy. Based on existing information on such molecules, it should be possible to determine from their expression on the isolated cell, the metastatic potential of the tumor via analysis of the circulating cells.

It is an object of the invention to provide kits for any cancer for which specific markers are known. A list summarizing tumor diathesis associated molecules and the usefulness and/or indication follows:

I Indicative of tumor origin

Muc-1——breast

PSA, PSMA——prostate

CEA——colon

CYPRA 21-1 ——lung

CA 125 ——ovarian

cytokeratins——see list

anti-HI67

II Cell cycle

nucleic acid dye

cyclin A, C & E

p27

III Cell viability/apoptosis

bax

Bcl-2

Caspase 7

Caspase 8

Caspase 9

Fas (CD95)

Cytochrome c

amexin V

anti-metal loproteinases

IV Drug sensitivity

estrogen, progesterone & androgen receptors

HER-2/neu

V. Drug resistance

P-glycoprotein (MDR)

t-glutamylcysteine synthase

taxol-resistance-associated-gene-1-5

cis-diamminedichloroplatinum II resistance genes

thymidylate synthetase

protein kinase C

telomerase

VI. Staging

Lewis A

C

BRCA-1 BRCA-2

CA15.3 (Muc-1), CA 27.29, CA 19.9

LASA

p53

cathepsin D

ras oncogene

The following table provides different cytokeratin markers that may be used to assess tissue origin of cells isolated using the methods of the present invention.

TABLE XIII
CYTOKERATIN MARKERS

| Cytokeratin Number | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Adrenal Cortex | − | − | − | − | − | − | − | + | − | − | − | − | − | − | − | − | − | + | + | − |
| Endometrium | − | − | − | − | − | − | + | + | − | − | − | − | − | − | − | − | − | + | + | − |
| Esophagus | − | − | − | + | − | − | − | + | − | − | − | − | + | − | − | − | − | + | + | − |
| Gastro-Intestinal | − | − | − | − | − | − | − | + | − | − | − | − | − | − | − | − | − | + | + | + |
| Kidney | − | − | − | − | − | − | + | + | − | − | − | − | − | − | − | − | + | + | + | − |
| Liver | − | − | − | − | − | − | + | + | − | − | − | − | − | − | − | − | − | + | + | − |
| Lung Columnar | − | − | − | − | − | − | + | + | − | − | − | − | − | − | − | − | − | + | + | + |
| Lung Basal | − | − | − | − | + | − | − | − | − | − | − | − | − | + | + | − | + | − | − | − |
| Mammary Gland Luminal | − | − | − | − | − | − | + | + | − | − | − | − | − | − | − | − | − | + | + | − |
| Mammary Gland Basal | − | − | − | − | + | − | − | − | − | − | − | − | − | + | − | − | + | − | − | − |
| Mesothelium | − | − | − | − | + | − | + | + | − | − | − | − | − | − | − | − | − | + | + | − |
| Oral | − | − | − | − | − | − | − | − | − | − | − | − | − | − | − | + | − | − | − | − |
| Ovary | − | − | − | − | − | − | + | + | − | − | − | − | − | − | − | − | − | + | + | − |
| Pancreas | − | − | − | + | + | − | + | + | − | − | − | − | − | + | − | − | + | + | + | − |
| Pituitary Endocrine cells | − | − | − | − | − | − | − | + | − | − | − | − | − | − | − | − | − | + | − | − |
| Pituitary Follicular | − | − | − | − | − | − | + | − | − | − | − | − | − | − | − | − | − | − | + | − |
| cells | | | | | | | | | | | | | | | | | | | | |
| Prostate Basal | − | − | − | − | + | + | − | + | − | + | − | − | + | + | − | − | + | + | + | − |
| Prostate Luminal | − | − | − | − | − | − | − | + | − | − | − | − | − | − | − | − | − | + | + | − |
| Skin | − | − | − | − | − | − | − | − | − | − | − | − | − | − | − | − | − | − | − | − |
| Testis | − | − | − | − | − | − | − | + | − | − | − | − | − | − | − | − | − | + | − | − |
| Thymus | − | − | − | − | + | − | − | − | − | + | − | − | − | − | − | − | − | + | − | + |
| Thyroid | − | − | − | + | − | − | − | + | − | + | − | − | + | − | − | − | − | + | + | − |
| Urinary Bladder | − | − | − | + | + | − | + | + | − | − | − | − | + | − | − | − | − | + | − | + |
| Uterine Cervix | − | − | − | − | + | + | + | + | − | − | − | − | − | + | + | + | + | + | + | − |
| Non-Epithelial: | | | | | | | | | | | | | | | | | | | | |
| Mammary adenocarcinoma | − | − | − | − | − | − | + | + | − | − | − | − | − | − | − | − | − | + | + | − |
| Prostate adenocarcinoma | − | − | − | − | − | − | − | + | − | − | − | − | − | − | − | − | − | + | + | − |
| Pancreatic adenocarcinoma | − | − | − | − | − | − | + | + | − | − | − | − | − | − | − | − | − | + | + | + |
| Gastro-Intestinal | − | − | − | − | − | − | + | + | − | − | − | − | − | − | − | − | − | + | + | + |
| adenocarcinoma | | | | | | | | | | | | | | | | | | | | |
| Endometrium adenocarcinoma | − | − | − | − | − | − | + | + | − | − | − | − | − | − | − | − | − | + | + | − |
| Lung adenocarcinoma | − | − | − | − | − | − | + | + | − | − | − | − | − | − | − | − | − | + | + | − |
| Lung SCC | − | − | − | + | − | − | − | + | − | − | − | − | + | + | + | + | + | + | − | − |
| Liver | − | − | − | − | − | − | + | + | − | − | − | − | − | − | − | − | − | + | + | − |
| Kidney renal cell tumor | − | − | − | − | − | − | + | + | − | − | − | − | − | − | − | − | + | + | + | − |
| Oral SCC | − | − | + | + | − | + | − | − | − | + | − | − | + | − | − | − | − | − | + | − |
| Ovary | − | − | − | + | + | − | + | + | − | + | − | − | + | − | − | − | − | + | + | − |
| Pituitary adenoma | − | − | − | − | − | − | − | + | − | − | − | − | − | − | − | − | − | + | − | − |
| Testis | − | − | − | − | − | − | − | + | − | − | − | − | − | − | − | − | − | + | + | − |
| Thyroid | − | − | − | + | − | − | − | + | − | + | − | − | + | − | − | − | − | + | + | − |
| Urinary Bladder | − | − | − | − | − | − | + | − | − | − | − | − | − | + | − | − | − | − | − | + |
| Uterine cervix | − | − | − | − | − | − | + | + | − | − | − | − | − | + | − | − | + | + | + | − |
| Valvular carcinoma | − | − | − | − | − | − | − | − | − | + | − | − | + | + | − | − | − | − | − | − |

The following demonstrates how the practice of the methods of the invention is facilitated by means of a kit for use in detection of circulating breast cancer cells:

As described above, the kit starts with reagents, devices and methodology for enriching tumor cells from whole blood. An exemplary kit for detecting breast cancer cells would contain that will assess six factors or indicators. The analytical platform needs to be configured such that the reporter molecules DAPI, CY2, CY3, CY3.5, CY5, and CYS.5 will be discriminated by the appropriate excitation and emission filters. The analytical platform in this example uses a fluorescent microscope equipped with a mercury arc lamp, and the appropriate filter sets for assessing the wavelengths of the detection labels employed. All of the markers are introduced at one time with this method. DAPi, which is excited with UV light, stains nucleic acids, and will be used to determine the nuclear morphology of the cell. CAM 5.2 labeled with CY2 will be used to stain the control cells. CY3 labeled α-cytokeratin will be used to label cytokeratins 7, 8, 18, and 19. An antibody conjugated with CY3.5 will be used to label HER-2/neu. An antibody conjugated with CY5 will be used to label Muc-l. An antibody conjugated to CY5.5 will be used to label estrogen receptors. By using the appropriate excitation and emission filters, the cancer cells will be identified. Again, the use of a Cell Tracks or Cell Spotter® cartridge is also envisioned in the method described.

Examples of different types of cancer that may be detected using the compositions, methods and kits of the present invention include apudoma, choristoma, branchioma, malignant carcinoid syndrome, carcinoid heart disease, carcinoma e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell and transitional cell reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, throphoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, antiokeratoma, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (Kaposi's, and mast-cell), neoplasms (e.g., bone, digestive system, colorectal, liver, pancreatic, pituitary, testicular, orbital, head and neck, central nervous system, acoustic, pelvic, respiratory tract, and urogenital), neurofibromatosis, and cervical dysplasia.

The present invention is not limited to the detection of circulating epithelial cells only. Endothelial cells have been observed in the blood of patients having a myocardial infarction. Endothelial cells, myocardial cells, and virally infected cells, like epithelial cells, have cell type specific determinants recognized by available monoclonal antibodies. Accordingly, the methods and the kits of the invention may be adapted to detect such circulating endothelial cells. Additionally, the invention allows for the detection of bacterial cell load in the peripheral blood of patients with infectious disease, who may also be assessed using the compositions, methods and kits of the invention.

Several citations to journal articles, U.S. Patents and U.S. Patent applications are provided hereinabove. The subject matter of each of the foregoing citations is incorporated by reference in the present specification as though set forth herein in full.

While certain of the preferred embodiments of the present invention have been described and specifically exemplified above, it is not intended that the invention be limited to such embodiments. Various modifications may be made thereto without departing from the spirit of the present invention, the full scope of which is delineated in the following claims.

What is claimed is:

1. A method for assessing a patient for the presence of a malignancy, comprising:
a) obtaining a biological specimen from a patient, said specimen comprising a mixed cell population suspected of containing hematopoietic and non-hematopoietic malignant cells;
b) preparing a sample wherein said biological specimen is mixed with a detectably labeled ligand which reacts specifically with the malignant cells, to the substantial exclusion of other sample components;
c) contacting said sample with at least one reagent which also specifically labels said malignant cells;
d) analyzing said sample to determine the presence and number of labeled cells, detection of said cells indicating the presence of malignancy, the greater the number of cells present, the greater the severity of the malignancy; wherein the method further comprises assessment of said labeled cells for alterations in at least one tumor diathesis-associated molecule.

2. The method of claim 1, wherein as an intermediary step between step c) and step d) the sample is contacted with an agent that detectably labels hematopoietic cells.

3. The method of claim 2 wherein said assessment of said tumor diathesis associated molecule comprises contacting said molecule with a detectably labeled agent having binding affinity for said molecule.

4. The method of claim 3, wherein said ligand is coupled to a first detectable label, said reagent is coupled to a second detectable label, said hematopoietic cells are labeled with a third detectable label and said tumor diathesis associated molecule is contacted with an agent coupled to a fourth detectable label, said first, second, third and fourth detectable labels being different.

5. The method as claimed in claim 1, wherein said labeled malignant cells are analyzed by a process selected from the group consisting of multiparameter flow cytometry, immunofluorescent microscopy, laser scanning cytometry, bright field base image analysis, capillary volumetry, spectral imaging analysis manual cell analysis, Cell Spotter® analysis, Cell Tracks analysis and automated cell analysis.

6. The method as claimed in claim 1, wherein said method is applied to detect and enumerate residual cancer cells in said biological specimen and said cells are further analyzed for alterations in at least one predetermined tumor diathesis-associated molecule following at least one tumor eradication procedure.

7. The method as claimed in claim 1, wherein said biological specimen is obtained from said patient periodically and assessed for the presence and number of circulating cancer cells and said cells are further analyzed for alterations in at least one predetermined tumor diathesis-associated molecule as an indicator of progression of said malignancy.

8. The method as claimed in claim 1, wherein said labeled malignant cells are contacted with chemotherapeutic agents to assess sensitivity thereto.

9. The method as claimed in claim 1, wherein said sample is an immunomagnetic sample comprising said biological specimen mixed with magnetic particles coupled to said ligand and wherein as an intermediate step between preparation of said immunomagnetic sample and contacting said immunomagnetic sample with at least one reagent, said immunomagnetic sample is subjected to a magnetic field to produce an enriched malignant cell suspension as the immunomagnetic sample.

10. The method as claimed in claim 9, wherein the volume of said immunomagnetic sample containing said enriched malignant cells is reduced relative to the volume of the original biological specimen.

11. The method as claimed in claim 9, wherein said magnetic particles are colloidal.

12. The method as claimed in claim 9, wherein said ligand is a monoclonal antibody specific for at least one cancer cell determinant, and said at least one reagent comprises at least one additional monoclonal antibody specific for a second cancer cell determinant and a third monoclonal antibody specific for an antigen present on a non tumor-cell, and said method further comprises adding to said labeled cancer cell-containing fraction a cell specific dye to allow exclusion of residual non-nucleated cells and cell debris from analysis.

13. The method as claimed in claim 12, wherein said ligand binds specifically to an epithelial cell adhesion molecule on said malignant cell.

14. The method as claimed in claim 12, wherein said one or more reagent binds specifically to an intracellular cytokeratin.

15. The method of claim 1, wherein said tumor diathesis-associated molecule is at least one of the molecules set forth in Table XII.

16. The method of claim 1, wherein said altered tumor diathesis-associated molecules are proteins present on individual cells and are assessed by a method selected from the group consisting of Cell Spotter® cytometry, Cell Tracks cytometry, immunofluorescence, mass spectometry and flow cytometry.

17. The method of claim 1, further comprising isolation and culturing of malignant cells isolated from said biological specimen.

18. The method of claim 17, wherein said cultured malignant cells are expanded from single cell colonies to generate clonal populations of said malignant cells.

19. The method of claim 18, wherein said clonal populations of cells are assessed for alterations in tumor diathesis associated molecules.

20. A malignant cell isolated from the cultured cells obtained from the method of claim 17.

21. A tumor vaccine comprising a cell, or a component thereof, as claimed in claim 20.

22. An altered diathesis associated molecule isolated from the cells of claim 19.

23. A tumor vaccine comprising the altered diathesis molecule of claim 19 or a fragment thereof.

24. The method of claim 17, further comprising contacting the cells with a therapeutic agent to assess sensitivity thereto.

25. The method of claim 22, wherein proteinaceous tumor diathesis-associated molecules present in said cultured cells are analyzed by a method selected from the group consisting of protein gel electrophoresis, column chromatography, HPLC, FPLC, immunohistochemistry, histochemistry and western blotting.

26. The method of claim 1, wherein said altered tumor diathesis-associated molecules are nucleic acids and are assessed by at least one method selected from the group consisting of library amplification of nucleic acids, polymerase chain reaction, agarose gel electrophoresis, Southern blotting, and Northern blotting.

27. The method of claim 26, wherein said library amplification is performed on genetic material isolated from an individual cell and said amplified genetic material is subjected to gene specific probing.

28. The method of claim 26 wherein said library amplification is performed on genetic material isolated from a plurality of malignant cells and said amplified genetic material is subjected to gene specific probing.

29. A method for assessing a patient for the presence of a non-hematopoietic malignancy, comprising:
a) obtaining a biological specimen from a patient, said specimen comprising a mixed cell population comprising hematopoietic and non-hematopoietic malignant cells;
b) preparing an immunomagnetic sample wherein said biological specimen is mixed with magnetic particles coupled to a ligand which reacts specifically with the malignant cells, to the substantial exclusion of other sample components;
c) separating said magnetic particle containing malignant cells from non-magnetic particle hematopoietic cells, and
d) analyzing said magnetic particle-containing cells to determine the presence and number of any malignant cells in said sample, detection of said cells indicating the presence of malignancy, the greater the number of cells present, the greater the severity of the malignancy; wherein the method further comprises assessment of said magnetic particle containing malignant cells for alterations in tumor diathesis-associated cellular molecules.

30. The method of claim 29, wherein said altered tumor diathesis-associated molecules are nucleic acids and are assessed by at least one method selected from the group consisting of library amplification of nucleic acids, polymerase chain reaction, agarose gel electrophoresis, Southern blotting, and Northern blotting.

31. The method of claim 30, wherein said library amplification is performed on genetic material isolated from an individual cell and said amplified genetic material is subjected to gene specific probing.

32. The method of claim 30 wherein said library amplification is performed on genetic material isolated from all malignant cells isolated and said amplified genetic material is subjected to gene specific probing.

33. The method of claim 1, wherein said altered tumor diathesis-associated molecules are carbohydrates and are assessed by a method selected from the group consisting of mass spectometry, lectin chromatography, and boronate affinity.

34. The method as claimed in claim 1, wherein said patient has been diagnosed with an epithelial cell carcinoma selected from the group consisting of prostate cancer, breast cancer, colon cancer, bladder cancer, ovarian cancer, renal cancer, head and neck cancer, pancreatic cancer and lung cancer.

35. The method of claim 34, wherein labeled cells are assessed for alterations in tumor diathesis-associated molecules selected from the group consisting of estrogen receptor, progesterone receptor, Her2/neu and MUC1.

36. The method of claim 34, wherein said labeled cells are assessed for alterations in tumor diathesis associated molecules selected from the group consisting of androgen receptor, PSA, uPA and PSMA.

37. The method of claim 34, wherein said labeled cells are assessed for alterations in tumor diathesis associated molecules selected from the group consisting of thymidylate synthase, EGFR, MUC2 and CEA-15.

38. The method of claim 34, wherein said labeled cells are assessed for alterations in tumor diathesis associated molecules selected from the group consisting of CEA-19-3, HCG, MDR, and MUC1.

39. The method of claim 34, wherein said labeled cells are assessed for alterations in tumor diathesis associated molecules selected from the group consisting of thymidylate synthase, MDR, Her-2/neu, and EGFR.

40. The method of claim 1, wherein said patient has stage I cancer.

42. The method of claim 1, wherein said patient has stage II cancer.

43. The method of claim 1, wherein said patient has stage III cancer.

44. The method of claim 1, wherein said patient has stage IV cancer.

45. The method as claimed in claim 29, wherein said patient has been diagnosed with an epithelial-derived cell carcinoma selected from the group consisting of prostate cancer, breast cancer, colon cancer, bladder cancer, ovarian cancer, renal cancer, head and neck cancer, pancreatic cancer and lung cancer.

46. The method of claim 45, wherein labeled cells are assessed for alterations in tumor diathesis-associated molecules selected from the group consisting of estrogen receptor, progesterone receptor, Her2/neu and MUC1.

47. The method of claim 45, wherein said labeled cells are assessed for alterations in tumor diathesis associated molecules selected from the group consisting of androgen receptor, PSA, uPA and PSMA.

48. The method of claim 45, wherein said labeled cells are assessed for alterations in tumor diathesis associated molecules selected from the group consisting of thymidylate synthase, EGFR, MUC2 and CEA-15.

49. The method of claim 45, wherein said labeled cells are assessed for alterations in tumor diathesis associated molecules selected from the group consisting of CEA-19-3, HCG, MDR, and MUC1.

50. The method of claim 45, wherein said labeled cells are assessed for alterations in tumor diathesis associated molecules selected from the group consisting of thymidylate synthase, MDR, Her-2/neu, and EGFR.

51. The method of claim 29, wherein said patient has stage I cancer.

52. The method of claim 29, wherein said patient has stage II cancer.

53. The method of claim 29, wherein said patient has stage III cancer.

54. The method of claim 29, wherein said patient has stage IV cancer.

55. A method for determining alterations in tumor diathesis associated molecules as a means to predict efficacy of therapy, comprising:
a) obtaining a sample from a patient;
b) isolating and enumerating circulating malignant cells from said sample if present, said method further comprising; and
c) determining the number of at least one predetermined tumor diathesis associated molecule on individual cells present in said sample as a means to predict efficacy of therapy.

56. A method for determining alterations in tumor diathesis associated molecules as a means to assess appropriate dosage for therapy, comprising:
a) obtaining a sample from a patient;
b) isolating and enumerating circulating malignant cells from said sample if present, said method further comprising; and
c) determining the number of at least one predetermined tumor diathesis associated molecule on individual cells present in said sample as a means to assess appropriate dosage for therapy.

57. A method for determining alterations in tumor diathesis associated molecules as a means to monitor efficacy of therapy, comprising:
a) obtaining a sample from a patient;
b) isolating and enumerating circulating malignant cells from said sample if present, said method further comprising; and
c) determining the number of at least one predetermined tumor diathesis associated molecule on individual cells present in said sample as a means to monitor efficacy of therapy.

58. The method of claim 57, wherein said samples are obtained from said patient, before, during or after administration of a therapeutic agent.

59. The method of claim 57, further comprising isolating and contacting said cells with a therapeutic agent to assess sensitivity thereto.

60. The method of claim 55, wherein said patient has breast cancer and said tumor diathesis associated molecule is Her-2/neu and said therapy is administration of anti-Her-2/neu antibody or a fragment thereof.

61. The method of claim 56, wherein said patient has breast cancer and said tumor diathesis associated molecule is Her-2/neu and said therapy is administration of anti-Her-2/neu antibody or a fragment thereof.

62. The method of claim 57, wherein said patient has breast cancer and said tumor diathesis associated molecule is Her-2/neu and said therapy is administration of anti-Her-2/neu antibody or a fragment thereof.

63. The method of claim 55, wherein said patient has breast cancer and said at least one tumor diathesis associated molecule comprises Her-2/neu and estrogen recepto r and said therapy is administration of anti-Her-2/neu antibody or a fragment thereof and tamoxifen.

64. The method of claim 56, wherein said patient has breast cancer and said at least one tumor diathesis associated molecule is Her-2/neu and estrogen receptor said therapy is administration of anti-Her-2/neu antibody or a fragment thereof and tamoxifen.

65. The method of claim 57, wherein said patient has breast cancer and said at least one tumor diathesis associated molecule is Her-2/neu and estrogen receptor and said therapy is administration of anti-Her-2/neu antibody or a fragment thereof and tamoxifen.

66. A method for determining alterations in tumor diathesis associated molecules as a means to assess cancer progression comprising:
a) obtaining a sample from a patient;
b) isolating and enumerating circulating malignant cells from said sample if present, said method further comprising; and
c) determining whether said cells contain a predetermined tumor diathesis associated molecule associated with a poor prognosis as a means to assess cancer progression.

67. The method of claim 66, wherein said tumor diathesis associated molecule is altered and is selected from the group consisting of Androgen Receptor, Cathepsin D, Estrogen Receptor, Estradiol, Progesterone Receptor, Somastatin, Steroid Receptor Coactivator-l (SRCl), Her-2 (cERB-b), EGFR, ras, c-fos, c-jun, c-myc, p53, p63, nm23/NDP Kinase, PTEN/MMAC1, SMAD4/DPC4, Notch-1, JAK3, Cyclin A, Cyclin B, Cyclin C, Cyclin D, Cyclin E, Ki67, MDR/MRP proteins, PSA, Prostatic Acid Phosphatase, CA 125, CA 15-3, CA 27-29, HGC, Cystic Fibrosis Transmembrane Regulator, Laminin Receptor, Neuron Specific Enolase (NSE), Alpha Fetoprotein, CD99/MIC2, DHEA, Prolactin, CD66e/CEA, Filaggrin, gp200 TAG72/CA72-4, UPA-receptor (CD87), Heregulin, IPO-38 Thymidylate Synthase, Topoisomerase Iia, Glutathione-S-Transferase (GST), Lung-Resistance related Protein/Major Fault Protein (LRP/MFP), and 06-Methylguanine-DNA methyltransferase (MGMT).

68. The method of claim 66, wherein said tumor diathesis associated molecule is aberrantly expressed relative to wild type expression and is selected from the group consisting of Androgen Receptor, Cathepsin D, Estrogen Receptor, Estradiol, Progesterone Receptor, Somastatin, Steroid Receptor Coactivator-1 (SRC1), Her-2 (cERB-b), EGFR, ras, c-fos, c-jun, c-myc, p53, p63, nm23/NDP Kinase, PTEN/MMAC1, SMAD4/DPC4, Notch-1, JAK3, Cyclin A, Cyclin B, Cyclin C, Cyclin D, Cyclin E, Ki67, MDR/MRP proteins, PSA, Prostatic Acid Phosphatase, CA 125, CA 15-3, CA 27-29, HGC, Cystic Fibrosis Transmembrane Regulator, Laminin Receptor, Neuron Specific Enolase (NSE), Alpha Fetoprotein, CD99/MIC2, DHEA, Prolactin, CD66e/CEA, Filaggrin, gp200 TAG72/CA72-4, UPA-receptor (CD87), Heregulin, IPO-38 Thymidylate Synthase, Topoisomerase Iia, Glutathione-S-Transferase (GST), Lung-Resistance related Protein/Major Fault Protein (LRP/MFP), and 06-Methylguanine-DNA methyltransferase (MGMT).

69. The method as claimed in claim 67, wherein said tumor diathesis associated molecule is a nucleic acid.

70. The method as claimed in claim 68, wherein said tumor diathesis associated molecule is a nucleic acid.

71. The method as claimed in claim 67, wherein said tumor diathesis associated molecule is a protein.

72. The method as claimed in claim 68, wherein said tumor diathesis associated molecule is a protein.

73. A method for performing a whole body biopsy on a patient, comprising:
a) obtaining a blood sample from a patient;
b) isolating and enumerating circulating non-hematopoietic malignant cells from said sample if present, said method further comprising; and
c) analyzing said cells for the presence and number of a panel of predetermined tumor diathesis associated molecules.

74. The method of claim 73, wherein said tumor diathesis associated molecule comprises at least two molecules selected from the group consisting of Androgen Receptor, Cathepsin D, Estrogen Receptor, Estradiol, Progesterone Receptor, Somastatin, Steroid Receptor Coactivator-l (SRCl), Her-2 (cERB-b), EGFR, ras, c-fos, c-jun, c-myc,p53, p63, nm23/NDP Kinase, PTEN/MMAC1, SMAD4/DPC4, Notch-1, JAK3, Cyclin A, Cyclin B, Cyclin C, Cyclin D, Cyclin E, Ki67, MDR/MRP proteins, PSA, Prostatic Acid Phosphatase, CA 125, CA 15-3, CA 27-29, HGC, Cystic Fibrosis Transmembrane Regulator, Laminin Receptor, Neuron Specific Enolase (NSE), Alpha Fetoprotein, CD99/MIC2, DHEA, Prolactin, CD66e/CEA, Filaggrin, gp200, TAG72/CA72-4, UPA-receptor (CD87), Heregulin, IPO-38, Thymidylate Synthase, Topoisomerase Iia, Glutathione-S-Transferase (GST), Lung-Resistance related Protein/Major Fault Protein (LRP/MFP), and 06-Methylguanine-DNA methyltransferase (MGMT).

75. The method as claimed in claim 73, wherein said patient has an epithelial cell cancer selected from the group consisting of prostate cancer, breast cancer, colon cancer, bladder cancer, ovarian cancer, renal cancer, uterine cancer, head and neck cancer, pancreatic cancer and stomach cancer.

76. A method for identifying alterations in a circulating tumor cells relative to cells present in a tumor mass in situ, comprising:
a) obtaining a biopsy specimen of said tumor mass from patient;
b) isolating circulating tumor cells from said patient, if present;
c) contacting said specimen and said isolated circulating tumor cells with a duplicate panel of agents which detect a plurality of tumor diathesis associated molecules;
d) detecting any tumor diathesis associated molecules present in said circulating tumor cells and in said specimen; and
e) determining whether said tumor diathesis associated molecules are altered in said circulating tumor cell relative to said biopsy specimen.

77. A test kit for screening a patient sample for the presence of a non-hematopoietic malignant cells comprising:
a) coated magnetic nanoparticles comprising a magnetic core material, a protein base coating material, and an antibody that binds specifically to a first characteristic determinant of said malignant cell, said antibody being coupled, directly or indirectly, to said base coating material;
b) at least one antibody having binding specificity for a second characteristic determinant of said malignant cell;
c) a cell specific dye for excluding sample components other than said malignant cells from analysis;
d) a device selected from the group consisting of a Cell Spotter® cartridge or a Cell Tracks cartridge; and
e) at least one detectably labeled agent having binding affinity for a tumor diathesis associated molecule.

78. A kit as claimed in claim 48, said kit further containing an antibody which has binding affinity for non-target cells, a biological buffer, a permeabilization buffer, a protocol and optionally, an information sheet.

79. A kit as claimed in claim 77 for screening patients for breast cancer, wherein said at least one detectably labeled agent having binding affinity for said tumor diathesis associated molecule is selected from the group consisting of MUC-1, estrogen, progesterone receptor, cathepsin D, p53, urokinase type plasminogen activator, epidermal growth factor, epidermal growth factor receptor, BRCA1, BRCA2, CA27.29, CA15.5, prostate specific antigen, plasminogen activator inhibitor and Her2-neu.

80. A kit as claimed in claim 77 for screening patients for prostate cancer, wherein said at least one detectably labeled agent having binding affinity for said tumor diathesis associated molecule is selected from the group consisting of prostate specific antigen, prostatic acid phosphatase, thymosin b-15, p53, HPC1 basic prostate gene, creatine kinase and prostate specific membrane antigen.

81. A kit as claimed in claim 77 for screening patients for colon cancer, wherein said at least one detectably labeled agent having binding affinity for said tumor diathesis associated molecule is selected from the group consisting of carcinoembryonic antigen, C protein, APC gene, p53, thymidylate synthase and matrix metalloproteinase (MMP-9).

82. A kit as claimed in claim 77 for screening patients with bladder cancer, wherein said at least one wherein said at least one detectably labeled agent having binding affinity for said tumor diathesis associated molecule is selected from the group consisting of nuclear matrix protein (NMP22),Bard Bladder tumor antigen (BTA), and fibrin degradation product (FDP).

83. A test kit as claimed in claim 77, wherein said at least one antibody comprises a panel of antibodies each having binding specificity for a different cancer cell characteristic determinant.
